SEQUENCE LISTING (10 sequences)

SEQ ID NO: 1 - protein, 223 aa, Artificial Sequence
  RBD monomer amino acid residues 319 to 541 of the SARS-CoV-2 Spike protein (Wuhan variant)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 673 aa, Artificial Sequence
  S1 subunit monomer amino acid residues 13 to 685 of the SARS-CoV-2 Spike protein (Wuhan variant)
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 219 aa, Artificial Sequence
  Amino acid residues 319 to 537 of the SARS-CoV-2 Spike protein RBD monomer of the B1.1.7 variant
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNK

SEQ ID NO: 4 - protein, 219 aa, Artificial Sequence
  Amino acid residues 319 to 537 of the SARS-CoV-2 Spike protein RBD monomer of the B.1.351 variant
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 5 - protein, 438 aa, Artificial Sequence
  fusion dimeric RBD variant SARS-CoV-2 antigen
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNK

SEQ ID NO: 6 - protein, 19 aa, Artificial Sequence
  Signal peptide
MGWSCIILFLVATATGVHS

SEQ ID NO: 7 - DNA, 1398 bp, Artificial Sequence
  fusion dimeric RBD variant SARS-CoV-2 antigen with Kozak sequence, signal peptide, histidine tag and stop codon
gccaccatgggctggtcctgcatcatcctgtttctggtggctaccgctaccggcgtgcacagtagagtgcagcctaccgagtctatcgtgcggttccccaacatcaccaacctgtgtcctttcggcgaggtgttcaacgccaccagattcgcctctgtgtacgcctggaaccggaagcggatctctaactgcgtggccgactactccgtgctgtacaactccgcctccttcagcaccttcaagtgctacggcgtgtcccctaccaagctgaacgacctgtgcttcaccaacgtgtacgccgactccttcgtgatcagaggcgacgaagtgcggcagatcgctcctggacagaccggcaatatcgccgactacaactacaagctgcccgacgacttcaccggctgtgtgatcgcttggaactccaacaacctggactccaaagtcggcggcaactacaattacctgtaccggctgttccggaagtccaacctgaagcctttcgagcgggacatctccaccgagatctaccaggctggcagcaccccttgtaatggcgtgaagggcttcaactgctacttcccactgcagtcctacggcttccagcctacctatggcgtgggctaccagccttacagagtggtggtgctgtccttcgagctgctgcatgctcctgctaccgtgtgcggccctaagaaatctaccaacctggtcaagaacaagcgggtgcagcccactgagagcattgtgcgcttccctaatatcacaaatctgtgccccttcggggaagtctttaatgctacccgcttcgcttccgtgtatgcttggaatagaaagcggatcagcaattgcgtcgccgattacagcgtcctgtacaatagcgccagcttctccacctttaagtgttatggcgtcagccccacaaagctcaacgatctctgttttaccaatgtctacgccgatagctttgtgattcgcggagatgaagtccgccagatcgcaccaggccagactggaaagatcgctgattacaattataagctccctgatgatttcacaggatgcgttatcgcctggaatagcaacaacctcgacagcaaagttggagggaattacaactacctctaccgcctcttcagaaagagcaacctcaagccatttgagagagacatcagtacagaaatctatcaggccggctctaccccttgcaacggcgtcgaggggtttaactgttactttcccctgcaatcttatgggtttcagcccacatacggcgtggggtatcaaccctatcgcgtggtggttctgagtttcgaactcctgcacgccccagccacagtgtgtggcccaaaaaagagcaccaatctcgttaagaacaagcaccatcaccatcaccattag

SEQ ID NO: 8 - DNA, 1314 bp, Artificial Sequence
  fusion dimeric RBD variant SARS-CoV-2 antigen
agagtgcagcctaccgagtctatcgtgcggttccccaacatcaccaacctgtgtcctttcggcgaggtgttcaacgccaccagattcgcctctgtgtacgcctggaaccggaagcggatctctaactgcgtggccgactactccgtgctgtacaactccgcctccttcagcaccttcaagtgctacggcgtgtcccctaccaagctgaacgacctgtgcttcaccaacgtgtacgccgactccttcgtgatcagaggcgacgaagtgcggcagatcgctcctggacagaccggcaatatcgccgactacaactacaagctgcccgacgacttcaccggctgtgtgatcgcttggaactccaacaacctggactccaaagtcggcggcaactacaattacctgtaccggctgttccggaagtccaacctgaagcctttcgagcgggacatctccaccgagatctaccaggctggcagcaccccttgtaatggcgtgaagggcttcaactgctacttcccactgcagtcctacggcttccagcctacctatggcgtgggctaccagccttacagagtggtggtgctgtccttcgagctgctgcatgctcctgctaccgtgtgcggccctaagaaatctaccaacctggtcaagaacaagcgggtgcagcccactgagagcattgtgcgcttccctaatatcacaaatctgtgccccttcggggaagtctttaatgctacccgcttcgcttccgtgtatgcttggaatagaaagcggatcagcaattgcgtcgccgattacagcgtcctgtacaatagcgccagcttctccacctttaagtgttatggcgtcagccccacaaagctcaacgatctctgttttaccaatgtctacgccgatagctttgtgattcgcggagatgaagtccgccagatcgcaccaggccagactggaaagatcgctgattacaattataagctccctgatgatttcacaggatgcgttatcgcctggaatagcaacaacctcgacagcaaagttggagggaattacaactacctctaccgcctcttcagaaagagcaacctcaagccatttgagagagacatcagtacagaaatctatcaggccggctctaccccttgcaacggcgtcgaggggtttaactgttactttcccctgcaatcttatgggtttcagcccacatacggcgtggggtatcaaccctatcgcgtggtggttctgagtttcgaactcctgcacgccccagccacagtgtgtggcccaaaaaagagcaccaatctcgttaagaacaag

SEQ ID NO: 9 - protein, 1273 aa, Human coronavirus
  MISC_FEATURE (1)..(1273): Spike protein sequence of the Wuhan-Hu-1 SARS-CoV-2 (UniProt No. P0DTC2)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 19 aa, Artificial Sequence
  Signal peptide.
MGWSLILLFLVAVATRVLS

(19) United States
(12) Patent Application Publication
(10) Pub. No.: US 2024/0316183 A1
(43) Pub. Date: Sep. 26, 2024

(54) SARS-COV-2 SUBUNIT VACCINE

(71) Applicants: HIPRA SCIENTIFIC, S.L.U., Amer (Girona) (ES); LABORATORIOS HIPRA, S.A., Amer (Girona) (ES)

(72) Inventors: Antonio BARREIRO VAZQUEZ, Amer (Girona) (ES); Antoni PRENAFETA AMARGOS, Amer (Girona) (ES); Laura FERRER SOLER, Amer (Girona) (ES); Luis GONZALEZ GONZALEZ, Amer (Girona) (ES); Ester PUIGVERT MOLAS, Amer (Girona) (ES); Jordi PALMADA COLOMER, Amer (Girona) (ES); Maria Teresa PRAT CABAÑAS, Amer (Girona) (ES); Carmen GARRIGA ALSINA, Amer (Girona) (ES)

(21) Appl. No.: 18/558,875

(22) PCT Filed: Apr. 25, 2022
(86) PCT No.: PCT/EP2022/060942

§ 371 Date: Nov. 3, 2023

(30) Foreign Application Priority Data
May 6, 2021 (EP) 21382411.3
Aug. 9, 2021 (EP) 21382750.4

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/545; A61K 2039/55566; A61K 2039/55572; A61K 2039/55577; A61K 2039/575; C12N 2770/20022; C12N 2770/20034

(57) ABSTRACT

The present invention relates to a protein subunit vaccine comprising at least one antigen characterized in that it comprises at least one monomer from at least one variant of severe acute respiratory syndrome coronavirus 2 (SARS-CoV-2), wherein the at least one monomer is selected from the group consisting of the S1 subunit of the Spike protein or the receptor-binding domain (RBD) of the Spike protein. In an aspect of the present invention, the protein subunit vaccine comprises at least one antigen characterized in that it comprises two monomers from at least one variant of SARS-CoV-2, wherein each of the monomers are selected from the group consisting of the S1 subunit or RBD protein, and wherein the monomers are chemically bound to each other, optionally through a linker, forming fusion dimers or non-fusion dimers. The protein subunit vaccine may further comprise at least an adjuvant and at least an immunostimulant.

### TECHNICAL FIELD OF THE INVENTION

The present invention is directed to a protein subunit vaccine comprising at least one antigen from at least one variant of severe acute respiratory syndrome coronavirus 2 (SARS-CoV-2), and optionally at least one adjuvant and at least one immunostimulant. The present invention is further directed to the use of said vaccine for generating an immunogenic and/or protective immune response against at least one variant of SARS-CoV-2 and kits comprising one or more doses of said vaccine.

### BACKGROUND OF THE INVENTION

Coronavirus disease 2019 (COVID-19), caused by a novel coronavirus named severe acute respiratory syndrome coronavirus 2 (SARS-CoV-2), was first reported in Wuhan, China in December 2019. Since then, COVID-19 has spread across the world and was declared a pandemic by the World Health Organisation (WHO) in March 2020. As of Feb. 8, 2021, 105 million people have been infected, and 2.3 million deaths have been recorded. SARS-CoV-2 is an enveloped virus carrying a single-stranded positive-sense RNA genome (˜30 kb), belonging to the genus Betacoronavirus from the Coronaviridae family. The virus RNA encodes four structural proteins including spike (S), envelope (E), membrane (M), and nucleocapsid (N) proteins, 16 non-structural proteins, and 9 accessory proteins. The S glycoprotein consists of an ectodomain (that can be processed into S1 and S2 subunits), a transmembrane domain, and an intracellular domain. Similar to the SARS-CoV, SARS-CoV-2 binds the human angiotensin-converting enzyme 2 (ACE2) via the receptor-binding domain (RBD) within the S1 subunit to facilitate entry into host cells, followed by membrane fusion mediated by the S2 subunit.

Development of a safe and effective COVID-19 vaccine is not easy, but manufacturing, distributing, and administering the vaccine could potentially face extraordinary challenges as well, especially in developing countries and if the vaccine must be injected, since the cold chain is required to maintain its stability and activity. Several vaccine strategies for COVID-19 are also intensively pursued, with Spike protein being the major target. These vaccines are produced from different platforms: RNA, DNA, recombinant proteins, viral vector-based, virus like particles (VLPs), live attenuated and inactivated viruses. These diverse types of vaccine candidates face a variety of challenges that are related to development, manufacturing, storage, and distribution to mass vaccination.

Subunit or recombinant protein vaccines use a whole protein, such as the Spike protein, or a protein fragment such as the S1, the RBD or fusion proteins as antigen. There are several advantages of subunit vaccines over other type of vaccines, such as they are cheap and easy to produce and more stable than other types of vaccines, such as vaccines based on mRNA or containing whole viruses or bacteria.

However, the main disadvantage of subunit vaccines is that the antigens used to elicit an immune response may lack molecular structures called pathogen-associated molecular patterns, which are common to a class of pathogen. These structures can be read by immune cells and recognized as danger signals, so their absence may result in a weaker immune response. Also, because these type of antigens do not infect cells, subunit vaccines mainly trigger antibody-mediated immune responses exclusively. Again, this means the immune response may be weaker than with other types of vaccines. To overcome this problem, subunit vaccines are sometimes delivered alongside with adjuvants. Therefore, subunit vaccines often require an adjuvant in the formulation to increase the immunogenicity.

Several adjuvants and immunostimulants have been developed or studied, such as aluminum salts, oil-in-water emulsions (MF59, AS03 and AF03), virosomes and AS04 as adjuvants and QS-21 or other saponins, monophosphoryl lipid A (MPLA), CpG (ODN) as immunostimulants. However, the selection of a proper adjuvant that helps promoting an appropriate immune response against a target pathogen at both innate and adaptative levels such that protective immunity can be elicited while maintaining the safety profile is critical and not straightforward. The selection of the wrong adjuvant may render a particular vaccine antigen inadequate. Thus, vaccine antigen selection must take into account adjuvant selection to avoid discarding potentially effective vaccine antigen candidates. Developing safe vaccines while obtaining a proper efficacy is still of paramount need.

Therefore, there is a need for novel, safe and effective vaccines against SARS-CoV-2, and particularly vaccines that offer an increased immunogenicity.

### DESCRIPTION OF THE FIGURES

FIG. 1. Titters of neutralizing antibodies against SARS-CoV-2 quantified by the pseudovirus neutralization assay (PBNA) for each Group of treatment. The log 10 of the dilution corresponding to the IC₅₀ is represented on the ordinates for each Group (A to I) on the abscissas.

FIG. 2A-H. Cytokine concentration in splenocytes cultures stimulated with the corresponding vaccine antigen (RBD or S1) per Group. FIG. 2A shows the IFN-γ concentration (pg/ml) on the ordinates for Groups A to E, on the abscissas. FIG. 2B shows the IL-4 concentration (pg/ml) on the ordinates for Groups A to E, on the abscissas. FIG. 2C shows the IL-6 concentration (pg/ml) on the ordinates for Groups A to E, on the abscissas. FIG. 2D shows the IL-10 concentration (pg/ml) on the ordinates for Groups A to E, on the abscissas. FIG. 2E shows the IFN-γ concentration (pg/ml) on the ordinates for Groups A and F to I, on the abscissas. FIG. 2F shows the IL-4 concentration (pg/ml) on the ordinates for Groups A and F to I, on the abscissas. FIG. 2G shows the IL-6 concentration (pg/ml) on the ordinates for Groups A and F to I, on the abscissas. FIG. 2H shows the IL-10 concentration (pg/ml) on the ordinates for Groups A and F to I, on the abscissas.

FIG. 3A-B. Comparison between anti-SARS-CoV-2 RBD total IgG antibody titres quantified by ELISA in convalescent and negative human serum samples (log 10 EC₅₀). The error bars represent the geometric mean and the geometric standard deviation (geometric SD). FIG. 3A shows the IgG antibody titre against RBD produced in HEK293 cells. FIG. 3B shows the IgG antibody titre against RBD produced in CHO cells.

FIG. 4. Grouped comparison between anti-SARS-CoV-2 RBD (produced in CHO cells) and anti-SARS-CoV-2 RBD (produced in HEK293 cells) total IgG antibody titres quantified by ELISA in convalescent human serum samples (log 10 EC₅₀). The error bars represent the geometric mean and the geometric standard deviation (geometric SD).

FIG. 5. Correlation between anti-SARS-CoV-2 RBD total IgG antibody titres (log 10 EC₅₀) represented on the ordinates and the elapsed days between the first positive PCR and the serum donation represented on the abscissas. Black squares: IgG antibody titre against RBD produced in CHO cells. Grey triangles: IgG antibody titre against RBD produced in HEK293 cells.

FIG. 6. Paired comparison between anti-SARS-CoV-2 RBD (produced in CHO cells) and anti-SARS-CoV-2 RBD (produced in HEK293 cells) total IgG antibody titres quantified by ELISA (log 10 EC₅₀), on the ordinates, for each convalescent human serum sample, on the abscissas. Each dot represents a single serum sample. Grey dots: IgG antibody titre against RBD produced in CHO cells. Black dots: IgG antibody titre against RBD produced in HEK293 cells.

FIG. 7: Anti-SARS-CoV-2 RBD IgG antibody titers (Log 10 EC₅₀) are depicted on the ordinates for each Group (A to E) of treatment on the abscissas, (A) Anti-SARS-CoV-2 RBD IgG antibody titers (Log 10 EC₅₀) on day 18 of the study, (B) Anti-SARS-CoV-2 RBD IgG antibody titers (Log 10 EC₅₀) on day 30 of the study.

FIG. 8: Anti-SARS-CoV-2 RBD IgG antibody titers (Log 10 EC₅₀) after one dose administration of different vaccine formulations are depicted on the ordinates for each Group of treatment (A to I) on the abscissas.

FIG. 9: Anti-SARS-CoV-2 RBD IgG antibody titers (Log 10 EC₅₀) after a second dose administration of different vaccine formulations are depicted on the ordinates for each Group of treatment (A to I) on the abscissas.

FIG. 10: Anti-SARS-CoV-2 RBD IgG antibody titres (Log 10 Endpoint titre) are depicted on the ordinates for each Group (A to D) of treatment on the abscissas. (A) Anti-SARS-CoV-2 RBD IgG antibody titres (Log 10 Endpoint titre) on day 21 of the study, (B) Anti-SARS-CoV-2 RBD IgG antibody titres (Log 10 Endpoint titre) between days 35 and 37 of the study.

FIG. 11: Neutralizing antibody response against SARS-CoV-2 Wuhan-hu-1 variant by PBNA. Neutralizing antibody titres (Log 10 IC₅₀) between days 35 and 37 of study are depicted on the ordinates for each Group (A to D) of treatment on the abscissas.

FIG. 12: Neutralizing antibody response against multiple SARS-CoV-2 variants by PBNA. Neutralizing antibody titres (Log 10 IC₅₀) are depicted on the ordinates for the different SARS-CoV-2 pseudovirus variants depicted on the abscissas. The SARS-CoV-2 variants assessed are Wu-1 (Wuhan-Hu-1 original sequence), Alpha (UK; B.1.1.7) variant; Beta (South Africa; B.1.351) variant, Gamma (Brazil; P.1) variant, and Delta (India; B.1.617.2) variant. (A) Neutralizing antibody titers obtained from animals of Group D, (B) Neutralizing antibody titers obtained from animals of Group E, (C) Neutralizing antibody titers obtained from animals of Group F. LD dotted line represents the assay limit of detection. The neutralizing antibody titer against India variant (delta; B.1.617.2) was only determined for Group D (FIG. 12A).

FIG. 13: Mean rectal temperature (° C.) are depicted on the ordinates at different days of the study on the abscissas for each Group (A to C). (A) Mean rectal temperature one day before the administration of the first dose (Day −1), at the time of the administration of the first dose (Day 0), and at 4 hours, 6 hours, 1 day, 2 days and 3 days after first vaccination (Day 0+4 h, Day 0+6 h, Day 1, Day 2 and Day 3). (B) Mean rectal temperature one day before the administration of the second dose (Day 20), at the time of the administration of the second dose (Day 21), and at 4 hours, 6 hours, 1 day, 2 days and 3 days after the second dose (Day 21+4 h, Day 21+6 h, Day 22, and Day 23).

FIG. 14: Anti-SARS-CoV-2 neutralizing antibody titres (Log 10 IC₅₀) are depicted on the ordinates for each Group (A to C) of treatment on the abscissas. The neutralizing antibody titres are depicted for each different variant: Alpha variant (UK; B.1.1.7), Beta variant (South Africa; B.1.351), Gamma variant (Brazil; P1), and Delta variant (India, B.1.617.2).

FIG. 15: Survival rates per each day after the experimental infection for the different Groups of treatment (A to C) are depicted. The survival rate (%) is represented on the ordinates and the elapsed days after the experimental infection on the abscissas. Animals in Group A received a vaccine composition comprising 20 pg of the recombinant fusion dimeric RBD variant SARS-CoV-2 antigen; Animals in Group B received a vaccine composition comprising 10 pg of the recombinant fusion dimeric RBD variant SARS-CoV-2 antigen; Animals in Group C received a mock-vaccine comprising PBS.

### DESCRIPTION OF THE INVENTION

### General Definitions

It must be noted that, as used herein, the singular forms “a”, “an”, and “the”, include plural references unless the context clearly indicates otherwise. Further, unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “about” when referred to a given amount or quantity indicates that a number can vary between ±20% around its indicated value. Preferably “about” means±15% around its value, more preferably “about” means±10, 8, 6, 5, 4, 3, 2% around its value, or even “about” means±1% around its value, in that order of preference.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. Any of the aforementioned terms (comprising, containing, including, having), whenever used herein in the context of an aspect or embodiment of the present invention may be substituted with the term “consisting of”, though less preferred.

When used herein “consisting of” excludes any element, step, or ingredient not specified in the claim element. When used herein, “consisting essentially of” does not exclude materials or steps that do not materially affect the basic and novel characteristics of the claim.

The term “subtype” herein can be replaced with “species”. It includes strains, isolates, clades, lineages, linages, and/or variants of any severe acute respiratory syndrome coronavirus, namely SARS-CoV-2. The terms “strain” “clade”, “lineage or linage”, “isolate” and/or “variant” are technical terms, well known to the skilled person, referring to the taxonomy of microorganisms, that is, referring to all characterized microorganisms into the hierarchic order of Families, Genera, Species, Strains. While the criteria for the members of a Family is their phylogenetic relationship, a Genera comprises all members which share common characteristics, and a Species is defined as a polythetic class that constitutes a replicating lineage and occupies a particular ecological niche. The term “strain” or “clade” describes a microorganism, in the present invention. a virus, which shares common characteristics with other microorganisms, like basic morphology or genome structure and organization, but varies in biological properties, like host range, tissue tropism, geographic distribution, attenuation or pathogenicity. The term “variant” describes a microorganism, in the present invention, a virus, which replicates and introduces one or more new mutations into its genome which results in differences from the original virus. The term “lineage” or “linage” describes a cluster of viral sequences derived from a common ancestor, which are associated with an epidemiological event, for instance, an introduction of the virus into a distinct geographic area with evidence of onward spread. Lineages are designed to capture the emerging edge of a pandemic and are at a fine-grain resolution suitable to genomic epidemiological surveillance and outbreak investigation. The SARS-CoV-2 lineage nomenclature is described for example, in Rambaut A. et al. A dynamic nomenclature proposal for SARS-CoV-2 lineages to assist genomic epidemiology. Nat Microbiol. 2020; 5(11): 1403-1407. Thus, by “at least one variant of severe acute respiratory syndrome coronavirus 2 (SARS-CoV-2)” is meant at least one variant, strain, isolate, lineage or linage, and/or clade of the SARS-CoV-2 virus. Preferably, the term “variant”, in line with the previous definition, and also in line with the WHO website, specifically refers to different SARS-CoV-2 viral sequences with one or more mutations derived from the same ancestor or etiological virus, i.e., in this case, the SARS-CoV-2 virus. In this specific context, it is thus preferably understood that the terms “SARS-CoV-2 variant” or “SARS-CoV-2 linage or linage” does not include viral genomes from other viruses, such as SARS or MERS viruses nor viral genomes derived from said other viruses.

More preferably, the term “variant” or “linage” includes all SARS-CoV-2 viral sequences that encode for a Spike protein with a percentage of amino acid sequence identity of at least 90%, 91%, 92%, 93%, 94%, preferably of at least 95%, 96%, 97%, 98%, or 99% from the Spike protein of the reference strain SARS-CoV-2 Wuhan-Hu-1 (GenBank accession No QHD43416.1 or Uniprot ID: P0DTC2), when both Spike proteins are locally aligned, for example, by using Basic Local Alignment Search Tool (BLAST).

Also preferably, the term “variant” or “linage” includes all SARS-CoV-2 viral sequences that encode for a RBD of the Spike protein with a percentage of amino acid sequence identity of at least 85%, 86%, 87%, 88%, or 89% preferably of at least 90%, 91%, 92%, 93%, 94%, most preferably of at least 95%, 96%, 97%, 98%, or 99% from the RBD of the Spike protein of the reference strain SARS-CoV-2 Wuhan-Hu-1 (GenBank accession No QHD43416.1 or Uniprot ID: P0DTC2, amino acid residues 319 to 541), when both RBD proteins are locally aligned, for example, by using Basic Local Alignment Search Tool (BLAST).

The different variants of SARS-CoV-2 can be found in databases such as Emma B. Hodcroft. 2021. “CoVariants: SARS-CoV-2 Mutations and Variations of Interest” (covariants.org/variants) or O'Toole A. et al., 2020 “A dynamic nomenclature proposal for SARS-CoV-2 lineages to assist genomic epidemiology”, PANGO lineages (cov-lineages.org/).

The terms “sequence identity” or “percent identity” in the context of two or more nucleotide sequences, polypeptide sequences or proteins sequences refers to two or more sequences or subsequences that are the same (“identical”) or have a specified percentage of nucleotide or amino acid residues that are identical (“percent identity”) when compared and aligned for maximum correspondence with a second molecule, as measured using a sequence comparison algorithm (e.g., by a BLAST alignment, or any other algorithm known to persons of skill), or alternatively, by visual inspection. The “sequence identity” or “percent identity” can be determined by calculating the number of identical nucleotides or amino acids at the same positions in a nucleic acid, polypeptide or protein. Calculation of percent identity includes determination of the optimal alignment between two or more sequences. Alignment can take into account insertions and deletions (i.e. “gaps”) in each of the sequences to be tested, such as, without limitation, in the non-coding regions of nucleic acids and truncations or extensions of polypeptide sequences. Computer programs and algorithms such as the Basic Local Alignment Search Tool (BLAST) may be used to determine the percent identity. BLAST is one of the many resources provided by the U.S. National Center for Biotechnology Information. Because the genetic code is degenerate, and more than one codon can encode a given amino acid, coding regions of nucleic acids are considered identical if the nucleic acids encode identical polypeptides. Thus, percent identity could also be calculated based on the polypeptide encoded by the nucleic acid. Percent identity could be calculated based on full length consensus genomic sequences or on a fraction of the genomic sequence, such as for example without limitation on individual open reading frames (ORFs).

A protein or peptide of the present invention has substantial identity with another if, optimally aligned, there is an amino acid sequence identity of at least about 60% identity with a synthetic or naturally-occurring protein or with a peptide derived therefrom, usually at least about 70% identity, more usually at least about 80% identity, preferably at least about 90% identity, and more preferably at least about 95% identity, and most preferably at least about 98% or 100% identity. Identity means the degree of sequence relatedness between two polypeptides or two polynucleotides sequences as determined by the identity of the match between two strings of such sequences, such as the full and complete sequence. Identity can be readily calculated. While there exist a number of methods to measure identity between polypeptide sequences, the term “identity” is well known to skilled artisans.

“Percent (%) amino acid sequence identity” with respect to proteins, polypeptides, antigenic protein fragments, antigens and epitopes described herein is defined as the percentage of amino acid residues in a candidate sequence that are identical with the amino acid residues in the reference sequence (i.e., the protein, polypeptide, antigenic protein fragment, antigen or epitope from which it is derived), after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available computer software such as BLAST, ALIGN, or Megalign (DNASTAR) software. Those skilled in the art can determine appropriate parameters for measuring alignment, including any algorithms needed to achieve maximum alignment over the full-length of the sequences being compared.

The term “subject” or “host” as used herein is a living multi-cellular vertebrate organism, including, for example, humans and non-human mammals, including (non-human) primates, companion animals such as dogs and cats, and domestic animals such as horses, bovine species such as cattle and sheep, ferrets, porcine species such as pigs, piglets sows or gilts, and zoo mammals such as felids, canids and bovids. Thus, the term “subject” or “host” may be used interchangeably with the term “animal” or “human” herein. Typically, the “subject” is a human. A human can be, for example, a neonate (up to 2 months of age), an infant (birth to 2 years of age), a child (2 years to 14 years of age), a teenager (15 years to 18 years of age), an adult (above 18 years of age), or a senior adult (about 65 years of age or older).

An “immunological response” or “immune response” to an antigen or composition is the development in a subject of an innate, humoral and/or a cellular immune response to an antigen present in the composition of interest. The term “enhanced” when used with respect to an immune response against SARS-CoV-2 antigens, such as an antibody response (e.g., neutralizing antigen specific antibody response), a cytokine response, a CD8 T cell response (e.g., immunodominant CD8 T cell response), or a CD4 T cell response, refers to an increase in the immune response in a subject administered with a vaccine comprising at least one SARS-CoV-2 antigens relative to the corresponding immune response observed from a subject administered with a vaccine that does not comprise any SARS-CoV-2 antigens.

In the context of the present invention, the term “monomer” is used to preferably refer to, but not limited to, the Receptor Binding Domain (RBD) of the Spike protein or the S1 subunit, from any variant of the SARS-CoV-2 virus. In particular, the term “monomer”, as used herein, refers to any protein that comprises, consists, or consists essentially of SEQ ID NO: 1, 2, 3, or 4, or a sequence with at least 50%, 60%, 70%, 80%, 85%, 90%, 91%, 92%, 93%, 94%, 95%, 96%, 97%, 98%, 99% or 100% sequence identity over its full length with any of sequences SEQ ID NO: 1, 2, 3, or 4. A monomer has the capacity to form chemical bonds to at least one other monomer molecule to form a multimer, i.e. a dimer, a trimer, a tetramer, a pentamer, etc. A dimer is a multimer formed by two monomers, these monomers may be identical in its sequence or may be different.

By “antigen” or “immunogen” is meant a substance that induces a specific immune response in a host animal. The antigen or the immunogen may comprise a whole organism, killed, attenuated or live; a subunit or partial fragment of an organism; a recombinant vector containing an insert with immunogenic properties; a fragment of DNA capable of inducing an immune response upon presentation to a host animal; a polypeptide, a protein or a fragment thereof, an epitope, or any combination thereof. In the context of the present invention, by “antigen” is meant a protein that comprises or consists of at least one monomer. In the context of the present invention, by “antigen” is meant a protein that comprises or consists of at least one multimer. A multimer or antigen can comprise two monomers (dimer or dimeric antigen), three monomers (trimer or trimeric antigen), four monomers (tetramer or tetrameric antigen) or more. The terms “multimeric antigen” or “multimer antigen” are synonymous. In the particular case of an antigen that consists of two monomers (understood as two RBDs, two S1, or one of each) then the antigen is understood to be in the form of a dimer. It is noted that “dimeric antigen” and “antigen in the form of a dimer” are synonymous and are herein used interchangeably. In the context of the present invention, the two monomers of the dimeric antigen are chemically connected or bound to each other, optionally through a linker. By “bound to each other” is meant that the monomers of the dimer are chemically connected by very weak, weak, strong, or very strong bonds, for instance by covalent bonds, non-covalent bonds, disulfide bonds or peptide bonds.

In the present invention, two types of antigens in the form of dimers are described: the “non-fusion dimer” and the “fusion dimer”. A “non-fusion dimer” is herein understood as an antigen formed by two monomers, wherein the two monomers are bound to each other by reversible bonds, for instance, through intermolecular disulfide bonds formed between their cysteines, forming a non-fusion dimeric antigen. For example, a “non-fusion dimer” as referred herein would be two soluble RBD monomers that are produced within a cell after being transfected by a nucleic acid encoding the said RBD monomer, and that when the RBD monomers are released to the cell supernatant they interact with each other, for instance, by means of their free (unbound) cysteines, forming disulfide bonds, and thereby forming what it is referred herein as a “non-fusion dimer”. Importantly, the two monomers in the non-fusion dimer are not connected by peptide bonds nor are they part of a single polypeptide.

By “fusion dimer” is referred herein as to an antigen formed by two monomers, wherein the two monomers have been joined, one after the other, so that they are synthetized or translated as a single unit, and thus the two monomers of the fusion dimer are part of a single polypeptide. Thus, contrary to the monomers of the non-fusion dimer, the two monomers comprised in a fusion dimer are connected by peptide bonds, optionally through a linker.

Further, in the present invention, when referring to “dimeric antigen” or “antigen in the form of a dimer” it should be understood as encompassing both, the non-fusion and the fusion dimeric antigen described above. By “monomeric RBD antigen” or “RBD-monomer” is referred herein as an antigen that comprises or consists of one monomer, wherein the monomer is RBD. By “dimeric RBD antigen” or “RBD-dimer” is referred herein as an antigen that comprises or consists of two monomers bound to each other, wherein the monomers are RBD. If the “dimeric RBD antigen” is a non-fusion dimer, it is called herein “non-fusion dimeric RBD antigen” or “non-fusion RBD-dimer”. If the “dimeric RBD antigen” is a fusion dimer, then it is called “fusion dimeric RBD antigen” or “fusion RBD-dimer”. Unless it is specified that the “dimeric RBD antigen” is a “non-fusion dimeric RBD antigen” or a “fusion dimeric RBD antigen”, it is to be understood that “dimeric RBD antigen” encompasses both types, i.e., fusion and non-fusion dimers of RBD. By “monomeric S1 antigen” or “S1-monomer” is referred herein as an antigen that comprises or consists of one monomer, wherein the monomer is S1. By “dimeric S1 antigen” or “S1-dimer” is referred herein as an antigen that comprises or consists of two monomers bound to each other, wherein the monomers are S1. If the “dimeric S1 antigen” is a non-fusion dimer, it is called herein “non-fusion dimeric S1 antigen” or “non-fusion S1-dimer”. If the “dimeric S1 antigen” is a fusion dimer, then it is called “fusion dimeric S1 antigen” or “fusion S1-dimer”. Unless it is specified that the “dimeric S1 antigen” is a “non-fusion dimeric S1 antigen” or a “fusion dimeric S1 antigen”, it is to be understood that “dimeric S1 antigen” encompasses both types, i.e., fusion and non-fusion dimers of S1.

The presence of antigens in the body normally triggers an immune response. Thus, antigens are “targeted” by antibodies. “Epitope” refers to the specific antigenic determinant of an antigen. An epitope could comprise three amino acids in a spatial conformation which is unique to the epitope. Generally, an epitope consists of at least five such amino acids, and more usually consists of at least 8-10 such amino acids. Methods of determining the spatial conformation of such amino acids are known in the art.

A subunit vaccine is a vaccine that presents one or more antigens to the immune system without introducing pathogen particles, whole or otherwise. By “protein subunit vaccine” is referred herein as to specific isolated antigens from viral or bacterial pathogen. A “protein subunit vaccine” is also referred herein as to specific recombinant antigens from viral pathogen.

“SARS-CoV-2 Spike (S) protein” means one of the four structural proteins (spike (S), nucleocapsid (N), envelope (E) and membrane (M) proteins) of SARS-CoV-2 virus. With a size of about 180-200 kDa, the S protein consists of an extracellular N-terminus, a transmembrane (TM) domain anchored in the viral membrane, and a short intracellular C-terminal segment. The total length of SARS-CoV-2 S protein is about 1273 amino acids and consists of a signal peptide (amino acids 1-13) located at the N-terminus, the S1 subunit (13 to 685 residues), and the S2 subunit (686-1273 residues); the last two regions are responsible for receptor binding and membrane fusion, respectively. In the S1 subunit, there is an N-terminal domain (14-305 residues) and a receptor-binding domain (RBD, 319-541 residues); the fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) comprise the S2 subunit. Thus, by “S1” or “S1 subunit” or “S1 antigen” is meant the S1 subunit located on the spike protein of coronavirus (CoV), and by “RBD” or “RBD antigen” is meant the receptor-binding domain located on the spike protein of coronavirus (CoV).

An “immunogenic fragment” of an antigen according to the present invention is a partial amino acid sequence of the antigen or a functional equivalent of such a fragment that also acts as an antigen, that is detected and bound by antigen-specific antibody or B-cell receptor. An immunogenic fragment of an antigen is shorter than the complete antigen and is preferably between about 10, 50 or 100 and about 1000 amino acids long, more preferably between about 10, 50 or 30 and about 500 amino acids long, even more preferably between about 50 and about 250 amino acids long. A fragment of the RBD or S1 antigens includes amino acids having at least 15, 20 or 65 contiguous amino acid residues having at least about 70%, at least about 80%, at least about 90%, preferably at least about 95%, more preferably at least about 98% sequence identity with at least about 15, 20 or 65 contiguous amino acid residues of SEQ ID NO. 1, 3, 4 or SEQ ID NO. 2, respectively. Depending on the expression system chosen, the protein fragments may or may not be expressed in native glycosylated form.

A protein or fragment that “corresponds substantially to” a protein or fragment of the SARS-CoV-2 virus is a protein or fragment that has substantially the same amino acid sequence and has substantially the same functionality as the specified protein or fragment of the SARS-CoV-2 virus.

A protein or fragment that has “substantially the same amino acid sequence” as a protein or fragment of the SARS-CoV-2 virus typically has more than 90% amino acid identity with this protein or fragment. Included in this definition are conservative amino acid substitutions.

“Antibodies” as used herein are polyclonal and/or monoclonal antibodies or fragments thereof, including recombinant antibody fragments, as well as immunologic binding equivalents thereof, which are capable of specifically binding to the SARS-CoV-2 proteins and/or to fragments thereof. The term “antibody” is used to refer to either a homogeneous molecular entity or a mixture such as a serum product made up of a plurality of different molecular entities. Recombinant antibody fragments may, e.g., be derived from a monoclonal antibody or may be isolated from libraries constructed from an immunized non-human animal.

“Adjuvant” as used herein is a substance used to enhance the immune response. The word adjuvant is derived from Latin: adjuve, meaning “to help.” Many classes of compounds have been described as adjuvants including mineral salts, microbial products, emulsions, saponins, cytokines, polymers, microparticles, and liposomes. A variety of compounds with adjuvant properties currently exist, and they exert their functions through different mechanisms of action. Based on their mechanism of action, the adjuvants can be divided into delivery systems and immunostimulants (immune potentiator) (Apostólico J. et al. Adjuvants: Classification, Modus Operandi, and Licensing. J Immunol Res. 2016; 2016:1459394). Delivery system adjuvants can function as carriers to which antigens can be associated, also create local proinflammatory response that recruit innate immune response cells to the site of the injection. The role of the immunostimulant is to activate innate response through pattern-recognition receptors (PRRs) or directly (i.e. cytokines). In general, activation of PRRs by their agonists induces “Antigen Presenting Cells” (APC) activation/maturation and cytokine/chemokine production that ultimately leads to adaptive immune responses. Thus, “Immunostimulant” as used herein is a compound that stimulates the immune system by inducing activation or increasing activity of any of its components. The stimulation derives from the direct or indirect stimulatory effect of the immunostimulant upon the cells of the immune system itself. Immunostimulants may activate the immune response through pattern-recognition receptors (PRRs) or directly. Immunostimulants can be natural or synthetic compounds. Immunostimulants may be given by themselves to activate nonspecific defence mechanisms, or they may be administered with a vaccine to activate nonspecific defence mechanisms as well as heightening a specific immune response. Immunostimulants can be combined with antigens and other adjuvants.

By “EC₅₀” or “half maximal effective concentration” or “50% effective dilution” is referred herein as the concentration of antibodies in sera that gives half-maximal binding, 50% of its maximal effect observed. The EC₅₀ can be determined by direct and saturable binding of a dilution series to a target antigen. The EC₅₀ in pseudovirus based neutralization assay is the dilution at which the relative light units (RLUs) are reduced by 50% compared with the virus control wells after subtraction of the background RLUs in the control group. Methods to determine the EC₅₀ in pseudovirus based neutralization assay are known by the skilled artisan, such as the ones described in Nie J. et al. Establishment and validation of a pseudovirus neutralization assay for SARS-CoV-2. Emerg Microbes Infect. 2020 December; 9(1):680-686, Nie J. et al. Quantification of SARS-CoV-2 neutralizing antibody by a pseudotyped virus-based assay. Nat Protoc. 2020 November; 15(11):3699-3715, or Hu J. et al. Development of cell-based pseudovirus entry assay to identify potential viral entry inhibitors and neutralizing antibodies against SARS-CoV-2. Genes Dis. 2020 December; 7(4):551-557.

By “IC₅₀” or “half maximal inhibitory concentration” or “50% inhibitory dilution” is referred herein as the concentration of antibodies in sera required to inhibit 50% of an infection. The IC₅₀ can be determined by direct and saturable binding of a dilution series to a target antigen. The IC₅₀ in pseudovirus based neutralization assay is the dilution at which the relative light units (RLUs) are reduced by 50% compared with the virus control wells after subtraction of the background RLUs in the control group. Methods to determine the IC₅₀ in pseudovirus neutralization assay are known by the skilled artisan, such as the ones described in Nie J. et al. Establishment and validation of a pseudovirus neutralization assay for SARS-CoV-2. Emerg Microbes Infect. 2020 December; 9(1):680-686, Nie J. et al. Quantification of SARS-CoV-2 neutralizing antibody by a pseudotyped virus-based assay. Nat Protoc. 2020 November; 15(11):3699-3715, or Hu J. et al. Development of cell-based pseudovirus entry assay to identify potential viral entry inhibitors and neutralizing antibodies against SARS-CoV-2. Genes Dis. 2020 December; 7(4):551-557.

By “endpoint titre” or “end-point titre” is referred herein as the reciprocal of the highest dilution that gives a reading above the cut-off. The cut-off value is preferably two to three times the mean background or negative control reading, more preferably three times the mean background or negative control reading. The endpoint titre can be determined by direct and saturable binding of a dilution series to a target antigen in an ELISA assay. Methods to determine the endpoint titre in ELISA assays are known by the skilled artisan, such as the one described in Frey A. et al. A statistically defined endpoint titre determination method for immunoassays. J Immunol Methods. 1998 Dec. 1; 221(1-2):35-41.

“Linker peptide” as used herein is a short peptide sequence that is located between the two monomers of the fusion dimer. Linker peptides are placed to provide the two monomers comprised in the fusion dimer with movement flexibility. In the context of the present invention, the linker peptide has at least one amino acid residue, preferably at least two consecutive amino acid residues, optionally 3, 4, 5, 6, 7, 8, 9, 10, 11, 12, 13, 14, 15, 16, 17, 18, 19, 20 or more amino acid residues. The linker peptide includes flexible linkers, rigid linkers, and in vivo cleavable linkers.

### DESCRIPTION

The design and optimization of the antigen comprised in a vaccine that promotes an appropriate immune response against a target pathogen at both innate and adaptative levels is not straightforward. Although it may be that the most antigenic epitopes or proteins of a pathogen are known, the generation of vaccines, particularly protein subunit vaccines, still requires a fine tuning of said antigens in order to enhance their immunogenicity and avoid misfolded or low immunogenic forms of them that may drive the immune response towards the wrong direction. The selection of the wrong antigen may render a particular vaccine inefficient. Thus, antigen selection must be carefully considered to avoid discarding potentially effective vaccine candidates and to help with vaccine development and providing new solutions to fight against pandemic, such as COVID-19.

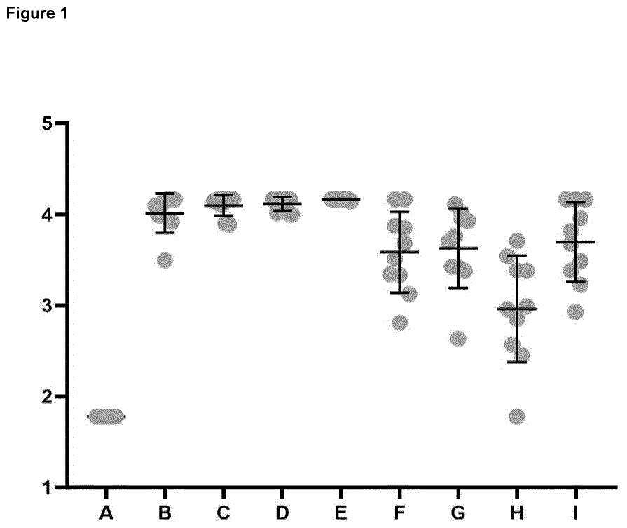

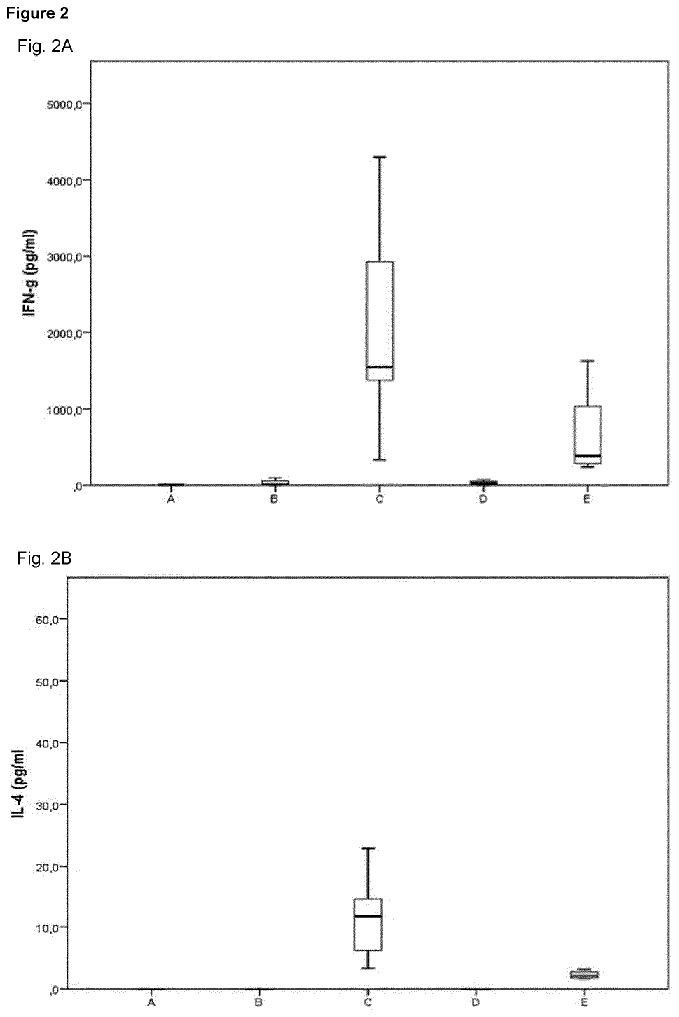

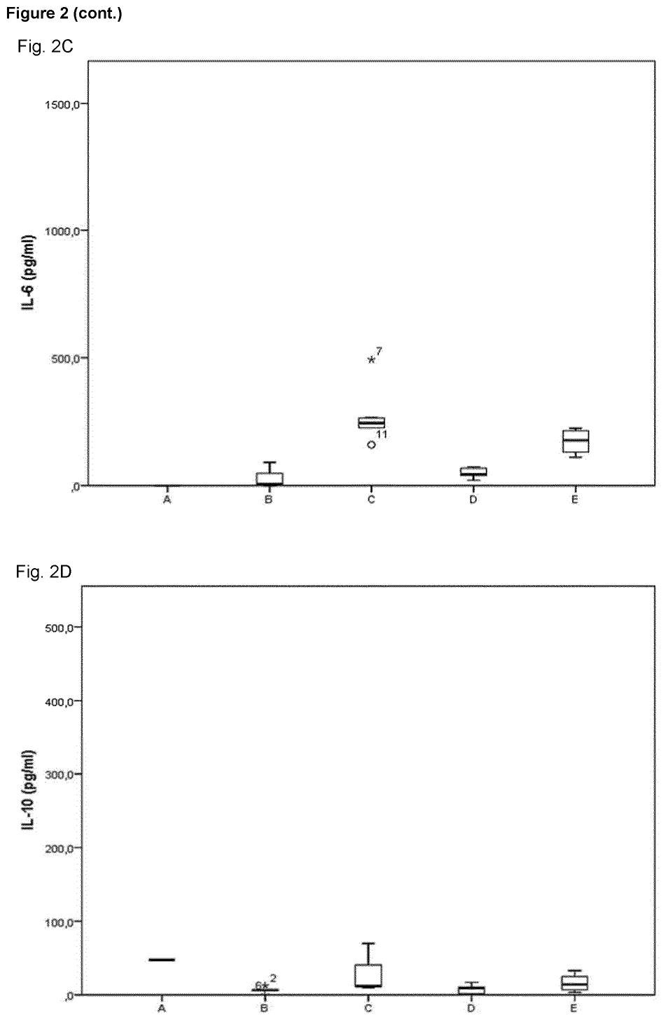

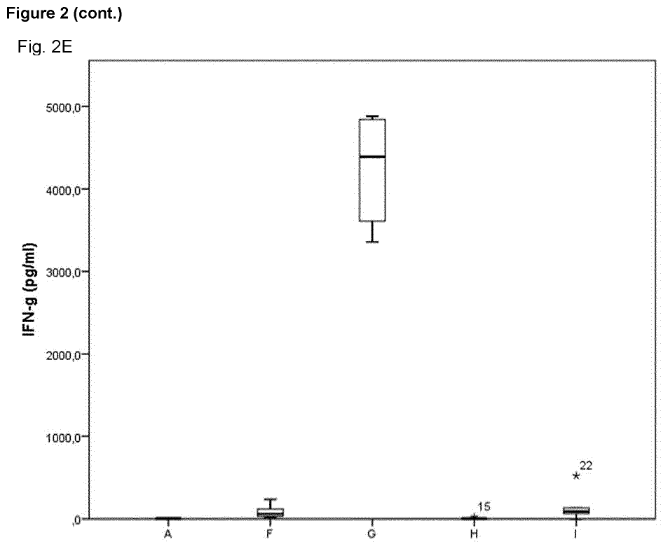

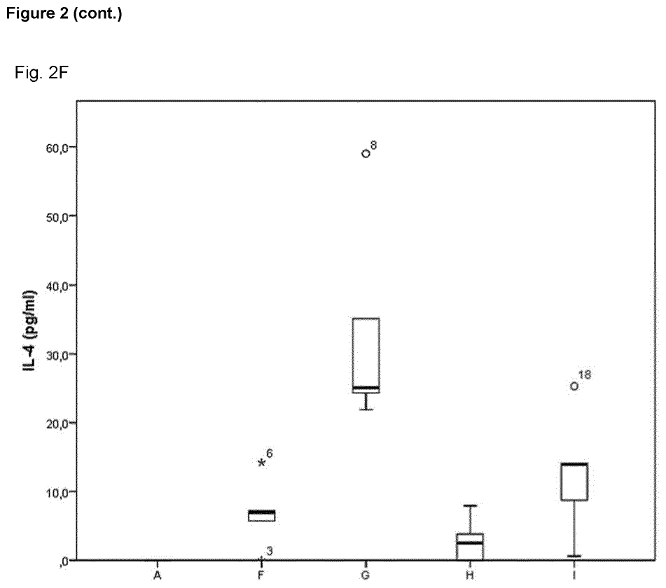

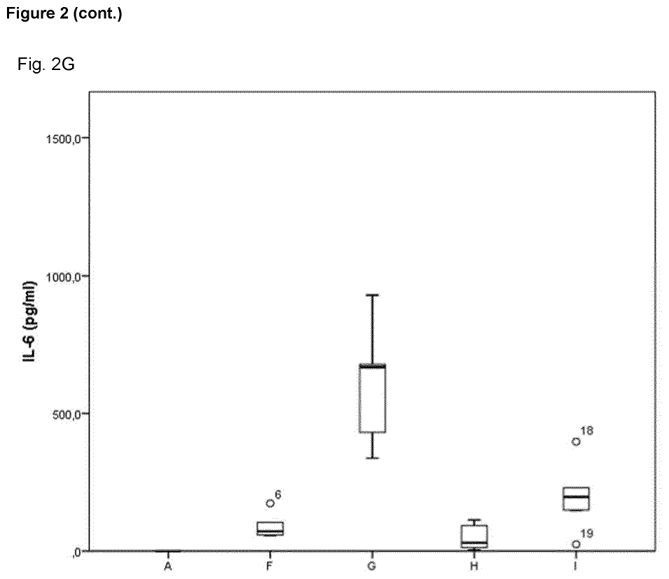

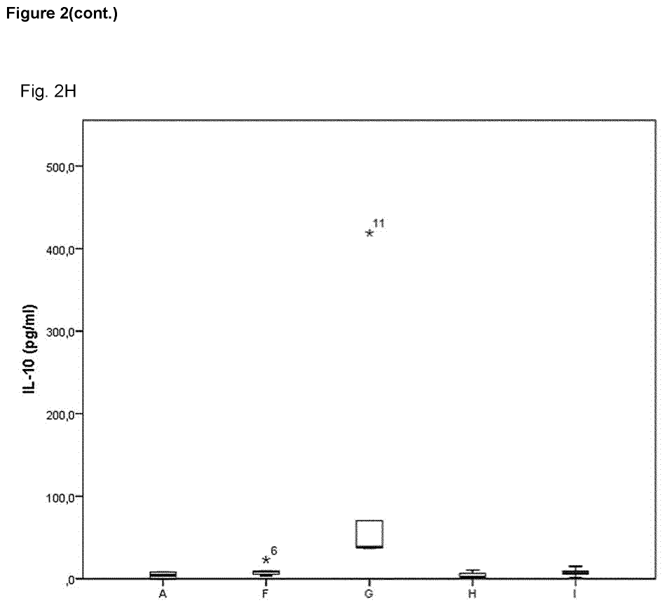

In the present invention, the inventors show herein, in FIGS. 1 and 2 and Example 2 that the RBD and the S1 subunit of SARS-CoV-2 virus have the ability of eliciting potent neutralizing antibodies and cellular immune responses, showing that they represent good candidates as a starting point towards the generation of a protein subunit vaccine against SARS-CoV-2 virus.

Thus, in a first aspect, the present invention relates to a protein subunit vaccine that comprises or consists of at least one antigen characterized in that it comprises or consists of at least one monomer from at least one variant of severe acute respiratory syndrome coronavirus 2 (SARS-CoV-2), wherein the at least one monomer is selected from the group consisting of the S1 subunit of the Spike protein or the receptor-binding domain (RBD) of the Spike protein, or any immunogenic fragment thereof.

In an embodiment, the at least one monomer comprised in the at least one antigen is a receptor-binding domain (RBD) of the Spike protein or an immunogenic fragment thereof. Preferably, the at least one monomer comprised in or consisting of the antigen is a recombinant receptor-binding domain (RBD) of the Spike protein or an immunogenic fragment thereof.

Preferably, said receptor-binding domain (RBD) of the Spike protein corresponds substantially to amino acid residues 319 to 541 of the SARS-CoV-2 Spike protein. Preferably, said receptor-binding domain (RBD) of the Spike protein comprises, consists, or consists essentially of SEQ ID NO: 1 or a sequence with at least 50%, 60%, 70%, 80%, 85%, 90%, 91%, 92%, 93%, 94%, 95%, 96%, 97%, 98%, 99% or 100% sequence identity over its full length with SEQ ID NO: 1.

In some embodiments, the receptor-binding domain (RBD) of the Spike protein comprises, consists, or consists essentially of SEQ ID NO:1.

Preferably, said receptor-binding domain (RBD) of the Spike protein corresponds substantially to amino acid residues 319 to 537 of the SARS-CoV-2 Spike protein. Preferably, said receptor-binding domain (RBD) of the Spike protein comprises, consists, or consists essentially of SEQ ID NO: 3 or a sequence with at least 50%, 60%, 70%, 80%, 85%, 90%, 91%, 92%, 93%, 94%, 95%, 96%, 97%, 98%, 99% or 100% sequence identity over its full length with SEQ ID NO: 3.

In some embodiments, the receptor-binding domain (RBD) of the Spike protein comprises, consists, or consists essentially of SEQ ID NO: 3.

Preferably, said receptor-binding domain (RBD) of the Spike protein comprises, consists, or consists essentially of SEQ ID NO: 4 or a sequence with at least 50%, 60%, 70%, 80%, 85%, 90%, 91%, 92%, 93%, 94%, 95%, 96%, 97%, 98%, 99% or 100% sequence identity over its full length with SEQ ID NO: 4. In some embodiments, the receptor-binding domain (RBD) of the Spike protein comprises, consists, or consists essentially of SEQ ID NO: 4.

In an embodiment, the at least one monomer comprised in the at least one antigen comprises or consists of the receptor-binding domain (RBD) and has at least 50%, 60%, 70%, 80%, 85%, 90%, 91%, 92%, 93%, 94%, 95%, 96%, 97%, 98%, 99% or 100% sequence identity over its full length with any of SEQ ID NO 1, SEQ ID NO 3 or SEQ ID NO 4.

In another embodiment, the at least one monomer comprised in the at least one antigen comprises or consists of the S1 subunit of the Spike protein or an immunogenic fragment thereof. Preferably, the at least one monomer is a recombinant S1 subunit of the Spike protein or an immunogenic fragment thereof. Preferably, the S1 subunit corresponds to the amino acid residues 13 to 685 of the SARS-CoV-2 Spike protein. More preferably, the S1 subunit corresponds to the amino acid residues 16 to 682 of the SARS-CoV-2 Spike protein.

Preferably, said S1 subunit of the Spike protein comprises, consists, or consists essentially of SEQ ID NO: 2 or a sequence with at least 50%, 60%, 70%, 80%, 85%, 90%, 91%, 92%, 93%, 94%, 95%, 96%, 97%, 98%, 99% or 100% sequence identity over its full length with SEQ ID NO: 2. In some embodiments, said S1 subunit of the Spike protein comprises, consists, or consists essentially of SEQ ID NO: 2.

In an embodiment, the at least one monomer comprised in the at least one antigen according to the first aspect or any of its embodiments is derived from the Wuhan SARS-CoV-2 variant. The Wuhan variant (Wuhan-Hu-1 seafood market pneumonia virus isolate) was the firstly described variant of SARS-CoV-2, which was found during the initial outbreak in Wuhan, China. The Spike protein of the Wuhan-Hu-1 consist of SEQ ID NO: 9 (UniProt No. P0DTC2). In another embodiment, the Wuhan variant comprises the mutation D614G in the Spike protein.

In another embodiment, the at least one monomer from at least one variant of SARS-CoV-2 comprised in the at least one antigen according to the first aspect or any of its embodiments is derived from a variant of concern (VOC) as defined by the Centers for Disease Control and Prevention (CDC) “SARS-CoV-2 Variant Classifications and Definitions”. The SARS-CoV-2 is observed to mutate, with certain combinations of specific point mutations proving to be more concerning than others. These mutations are the reason of the increased transmissibility, increased virulence, and possible emergence of escape mutations in new variants. The term “variant of concern” (VOC) is a designation used in newly emerged variants of SARS-CoV-2 with mutations that provide an increased transmissibility and/or morbidity and/or mortality and/or decreased susceptibility to antiviral or therapeutic drugs and/or have the ability to evade immunity and/or ability to infect vaccinated individuals, among others. As explained above, the term “variant” is preferably understood to refer to “lineage” or “linage”, i.e., to different viral sequences deriving from the same SARS-CoV-2 common ancestor. Therefore, preferably, the different “variant of concerns”, as referred herein, do not include viral sequences deriving from other viruses such as SARS or MERS.

In another embodiment, the at least one monomer from at least one variant of SARS-CoV-2 comprised in the at least one antigen according to the first aspect or any of its embodiments is derived from the United Kingdom SARS-CoV-2 variant VOC 202012/01 (Lineage B.1.1.7). According to WHO, on 14 Dec. 2020, authorities of the United Kingdom reported to WHO a variant referred to by the United Kingdom as SARS-CoV-2 VOC 202012/01 (Variant of Concern, year 2020, month 12, variant 01) also known as Lineage B.1.1.7 or 501Y.V1. This variant is described in the scientific literature, see, e.g., in Wise, J. Covid-19: New coronavirus variant is identified in UK. BMJ 2020, 371, m4857. This variant contains 23 nucleotide substitutions and is not phylogenetically related to the SARS-CoV-2 virus circulating in the United Kingdom at the time the variant was detected. Among the variant's several mutations there is one in the receptor-binding domain (RBD) of the spike protein that changes the asparagine at position 501 to tyrosine (N501Y). Another of the mutations in the VOC 202012/01 variant, the deletion at position 69/70del was found to affect the performance of some diagnostic PCR assays with an S gene target. As of 30 December, VOC-202012/01 variant has been reported in 31 other countries/territories/areas in five of the six WHO regions.

In another embodiment, the at least one monomer from at least one variant of SARS-CoV-2 comprised in the at least one antigen according to the first aspect or any of its embodiments is derived from the South African SARS-CoV-2 variant (Lineage B.1.351). On 18 December, national authorities in South Africa announced the detection of a new variant of SARS-CoV-2 that is rapidly spreading in three provinces of South Africa. South Africa has named this variant as Lineage B.1.351, also known as 501Y.V2. This variant is described in the scientific literature, see, e.g., Tegally et al. Emergence and rapid spread of a new severe acute respiratory syndrome-related coronavirus 2 (SARS-CoV-2) lineage with multiple spike mutations in South Africa. medRxiv 2020. SARS-CoV-2 South African variant is characterised by three mutations K417N, E484K and N501Y in the RBD. While SARS-CoV-2 VOC 202012/01 from the UK also has the N501Y mutation, phylogenetic analysis has shown that the virus from South Africa are different virus variants.

In another embodiment, the at least one monomer from at least one variant of SARS-CoV-comprised in the at least one antigen according to the first aspect or any of its embodiments is derived from the Brazilian SARS-CoV-2 variant VOC-202101/02 (Linage B.1.1.28). Brazilian variant is also known as Lineage P.1, also known as 20J/501Y.V3, Variant of Concern 202101/02 (VOC-202101/02). This variant is described in the scientific literature, see, e.g., Faria, et al. Genomic Characterisation of an Emergent SARS-CoV-2 Lineage in Manaus: Preliminary Findings. This variant of SARS-CoV-2 has 17 unique amino acid changes, ten of which are in its spike protein, including these three designated to be of particular concern: N501Y, E484K and K417T. This variant of SARS-CoV-2 was first detected by the National Institute of Infectious Diseases (NIID), Japan, on 6 Jan. 2021 in four people who had arrived in Tokyo having visited Amazonas, Brazil four days earlier. It was subsequently declared to be in circulation in Brazil and spreading around the world.

Recently, a California variant has been also known as Variant of Concern CAL.20C. Thus, in another embodiment, the at least one monomer from at least one variant of SARS-CoV-2 comprised in the at least one antigen according to the first aspect or any of its embodiments is derived from the Californian SARS-CoV-2 (Linage B.1.427 or B.1.429). This variant is characterized by the mutations S131, W152C in the N-terminal domain (NTD) of the spike protein and by the L452R mutation in the RBD of the spike protein. This variant was originally detected in California (Linage B.1.427 or B.1.429).

Many other variants have been described, such as the B.1.207 variant in Nigeria, which has a mutation in the spike protein (P681H) that is also found in the VOC 202012/01 variant, the B.1.617 variant in India (Linage B.1.617, Indian variant), which has the mutations P681R, E484Q and L425R in the spike protein, or the Danish variant, referred to as the “Cluster 5” variant by Danish authorities, and which has a combination of mutations not previously observed. The rising variants worldwide can be easily found by the skilled person, e.g., in the website databases such as disclosed by Emma B. Hodcroft. 2021. “CoVariants: SARS-CoV-2 Mutations and Variations of Interest.” (covariants.org/variants) or by O'Toole A. et al., 2020 “PANGO lineages” (cov-lineages.org/). It is thus to be understood that the present invention covers protein subunit vaccines comprising at least one antigen, characterized in that it comprises or consist of at least one monomer from at least one variant of SARS-CoV-2, wherein the monomer from at least one variant of SARS-CoV-2 comprised in the at least one antigen is derived from any strain or clade or variant or lineage or isolate of SARS-CoV-2.

The Omicron variant (Lineage B.1.1.529 or GR/484A, which, unless specifically indicated, it is considered to include all BA lineages (BA.1, BA.2, BA.3, BA.4, BA.5 and descendent lineages)) was first reported to WHO from South Africa on 24 Nov. 2021 and subsequently categorized as VOC. This variant has a large number of Spike substitutions, including A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F. Omicron variants as referred herein also include circulating recombinant variants such as BA.1/BA.2 lineages, known as XE. XE combines genetic material from the Omicron BA.1 and BA.2 lineages, along with three new mutations that are not present in either pre-existing strain.

The Delta variant (Lineage B.1.617.2 or G/478K.V1 and AY lineages) carries the Spike substitutions T19R, (V70F*), T95I, G142D, E156−, F157−, R158G, (A222V*), (W258L*), (K417N*), L452R, T478K, D614G, P681R, D950N. It was first identified in India and was categorized as VOC. Delta variants as referred herein also include circulating recombinant variants such as delta variant with BA.1 lineage, known as XD and XF. Both, XD and XF are recombinants of the genetical material from the delta and the BA.1 lineages.

Importantly, given the continuous evolution of the SARS-CoV-2 virus and the constant developments in our understanding of the impacts of variants, the working definitions and nomenclature used to refer to the different variants may be periodically adjusted. The established nomenclature systems for naming and tracking SARS-CoV-2 genetic lineages currently used in scientific research are by GISAID, Nextstrain and Pango. Thus, since the name of a variant may change in time, we provide below a table retrieved from WHO website, in which, as of 6 of Aug. 2021, the following variant nomenclature represents the established nomenclature to date, and thus it was the one used at the time of drafting the present application:

### Currently Designated Variants of Concern:

| | | | | Additional | | |
|---|---|---|---|---|---|---|
| | | | | amino acid | Earliest | |
| WHO | Pango | GISAID | Nextstrain | changes | documented | Date of |
| label | lineages | clade | clade | monitored* | samples | designation |
| Alpha | B.1.1.7 | GRY | 20I (V1) | +S: 484K | United | 18 Dec. 2020 |
| | | | | +S: 452R | Kingdom, | |
| | | | | | September 2020 | |
| Beta | B.1.351 | GH/501Y.V2 | 20H (V2) | +S: L18F | South Africa, | 18 Dec. 2020 |
| | B.1.351.2 | | | | May 2020 | |
| | B.1.351.3 | | | | | |
| Gamma | P.1 | GR/501Y.V3 | 20J (V3) | +S: 681H | Brazil, | 11 Jan. 2021 |
| | P.1.1 | | | | November 2020 | |
| | P.1.2 | | | | | |
| Delta | B.1.617.2 | G/478K.V1 | 21A | +S: 417N | India, | VOI: 4 Apr. |
| | AY.1 | | | | October 2020 | 2021 |
| | AY.2 | | | | | VOC: 11 May |
| | AY.3 | | | | | 2021 |

*Notable spike (S) amino acid changes under monitoring, which are currently reported in a minority of sequenced samples.

As of Apr. 12, 2022, the following variant nomenclature is also included at WHO website:

| | | | | Additional | | |
|---|---|---|---|---|---|---|
| | | | | amino acid | Earliest | |
| WHO | Pango | GISAID | Nextstrain | changes | documented | Date of |
| label | lineage• | clade | clade | monitored∘ | samples | designation |
| Delta | B.1.617.2 | G/478K.V1 | 21A, 21I, 21J | +S: K417N | India, | VOI: 4 Apr. |
| | | | | +S: E484K | October 2020 | 2021 |
| | | | | | | VOC: 11 |
| | | | | | | May 2021 |
| Omicron* | B.1.1.529 | GR/484A | 21K, 21L, 21M | +S: R346K | Multiple | VUM: 24 |
| | | | | +S: L452R | countries, | Nov. 2021 |
| | | | | +S: F486V | November 2021 | VOC: 26 |
| | | | | | | Nov. 2021 |

In view of these tables, and as a mode of example, the United Kingdom variant can also be referred to as variant B.1.1.7 or alpha variant; the South African variant can also be referred to as variant B.1.351 or beta variant; the Brazilian variant can also be referred to as variant P.1 or gamma variant; the Indian variant can also be referred to as variant B.1.617.2 or delta. The different names of each variant are considered synonymous and are herein used interchangeably. The different names and specific point mutations used to design the different SARS-CoV-2 variants can be easily retrieved and updated by the skilled person, see, e.g., the WHO website: who.int/en/activities/tracking-SARS-CoV-2-variants/.

In a particular embodiment, the at least one monomer from at least one variant of SARS-CoV-2 comprised in the at least one antigen according to the first aspect or any of its embodiments is derived from a SASR-CoV-2 variant selected from the group including, but not limited to, Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant), Linage B.1.1.7 (United Kingdom variant), Linage B.1.617.2 or G/478K.V1 (Delta Variant) or Linage B.1.1.529 or GR/484A (Omicron variant) or any combination thereof. In a preferred embodiment, the at least one monomer from at least one variant of SARS-CoV-2 comprised in the at least one antigen according to the first aspect or any of its embodiments is derived from a SASR-CoV-2 variant selected from the group consisting of Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant), Linage B.1.1.7 (United Kingdom variant), Linage B.1.617.2 or G/478K.V1 (Delta Variant) or Linage B.1.1.529 or GR/484A (Omicron variant) or any combination thereof.

In an embodiment, the at least one antigen may be in the form of a monomer or multimer, such as dimers, trimers, tetramers, pentamers, hexamers, heptamers, octamers, nonamers, or decamers, or any combinations thereof. In an embodiment, the at least one antigen consists of two monomers, and it is in the form of a dimer (dimeric antigen). In another embodiment, the protein subunit vaccine comprises a mixture of one antigen or more than one antigen that are present in different forms, such as monomers and dimers. In an embodiment, the protein subunit vaccine comprises at least one antigen in different forms, such as in monomeric and dimeric forms, wherein the monomers of said monomeric and dimeric forms are RBD (hereinafter called monomeric RBD antigen or RBD-monomer antigen and dimeric RBD antigens or RBD-dimer antigens, respectively, as defined in the definition section). In an embodiment, the protein subunit vaccine comprises a mixture of at least one monomeric RBD antigen and at least one dimeric RBD antigen. In an embodiment, the protein subunit vaccine comprises higher proportion of dimeric RBD antigens than monomeric RBD antigens. In an embodiment the antigen or antigens proportion comprised in the protein subunit vaccine is/are at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% dimeric RBD antigen/s. The calculation of the percentage of monomeric RBD antigen and dimeric RBD antigen can be determined by using standard methods, such as Size Exclusion Chromatography (SEC) or High-Performance Liquid Chromatography (HPLC). The area under the peak in the Size Exclusion Chromatography of the identified dimeric and monomeric peak represents the relative amount of the RBD-monomer and RBD-dimer. Obtaining a particular proportion of the dimeric RBD antigen over monomeric RBD antigen is known by the skilled in the art, by mixing for example different volumes of the dimeric RBD antigen and the monomeric RBD antigen. In an embodiment, the protein subunit vaccine comprises higher proportion of non-fusion dimeric RBD antigens than monomeric RBD antigens. In an embodiment, the protein subunit vaccine comprises a percentage of at least 51%, 55%, 60%, 65%, 70%, 75%, 80%, 85%, 90%, 95% of the total antigen comprised in the protein subunit vaccine is a dimeric RBD antigen.

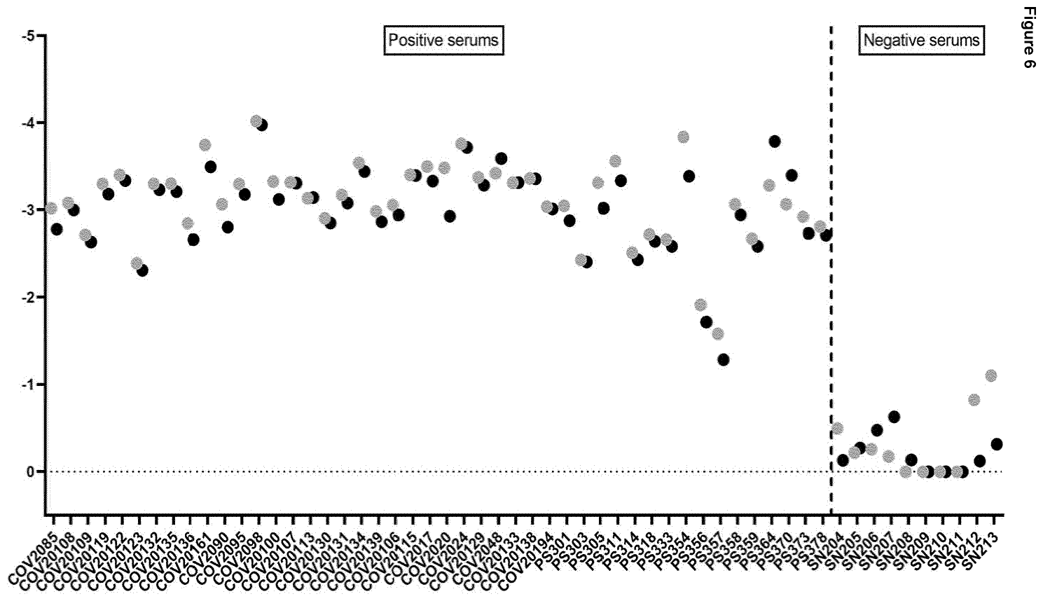

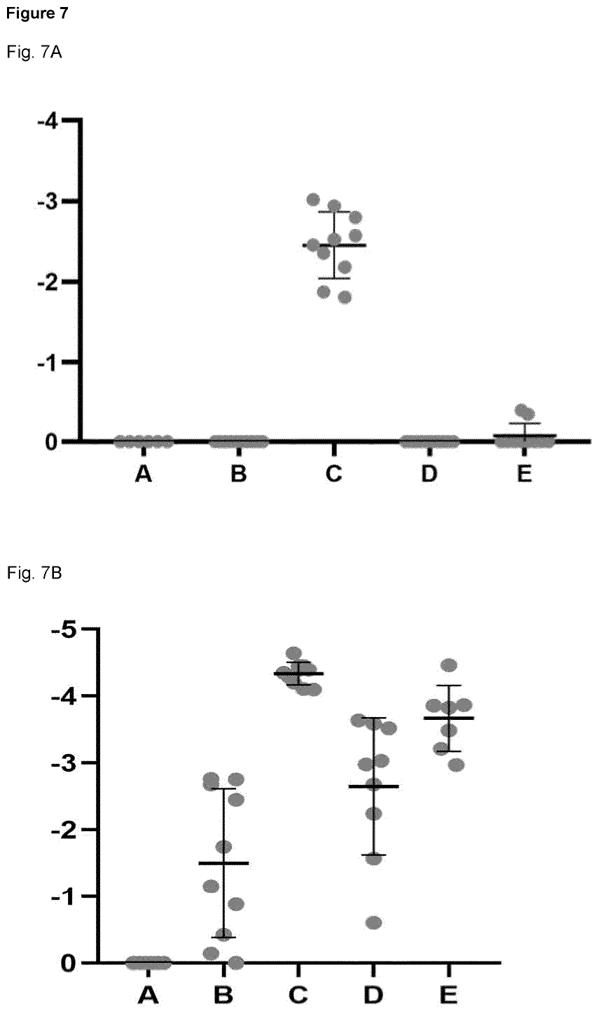

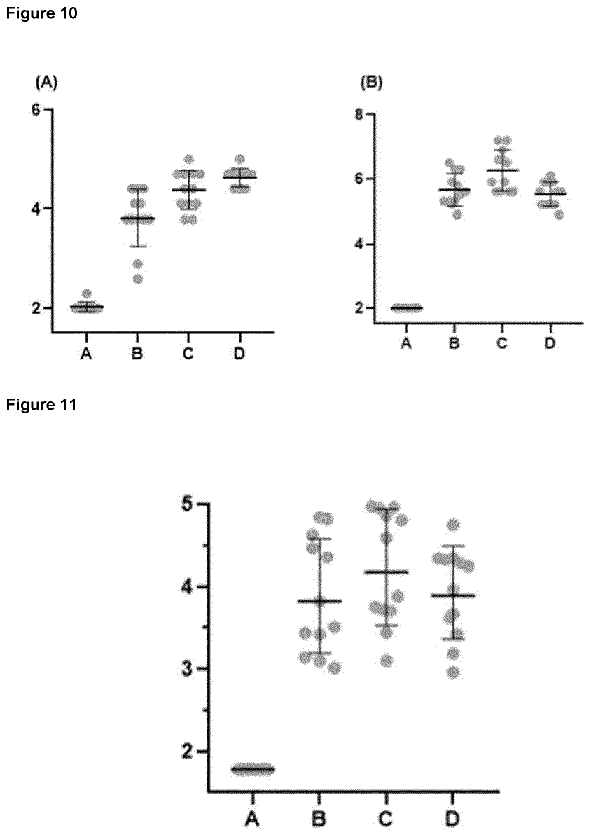

Further, the inventors of the present invention have also tested different vaccines formulations including different proportions of monomers and non-fusion dimers. The results are depicted in FIG. 7, where it is shown that, when the vaccine formulation has a higher proportion of the dimeric RBD antigen (56%) over the monomeric RBD antigen (44%) (Group E), the humoral response is significantly increased. That is, even if the vaccine composition does not comprise any immunostimulant and even if the vaccine comprises half dose of RBD antigen (10pg/dose) the humoral response is higher compared to the other groups (see FIG. 7E). The immunogenicity of this non-fusion dimer-based vaccine enriched in RBD dimers was also compared to that of a commercially available mRNA vaccine (Spikevax, COVID-19 mRNA Vaccine (Moderna Biotech Spain, S.L.)). FIG. 10 shows that after the second dose, high levels of IgG antibody titres were elicited in all groups, with a trend of higher levels in Group C, demonstrating the enhanced immunogenicity elicited by a vaccine comprising the non-fusion RBD dimeric:monomeric non-variant SARS-CoV-2 antigen, with a high proportion of dimeric RBD as antigen. This trend is also showed in FIG. 11, when neutralizing antibodies were measured.

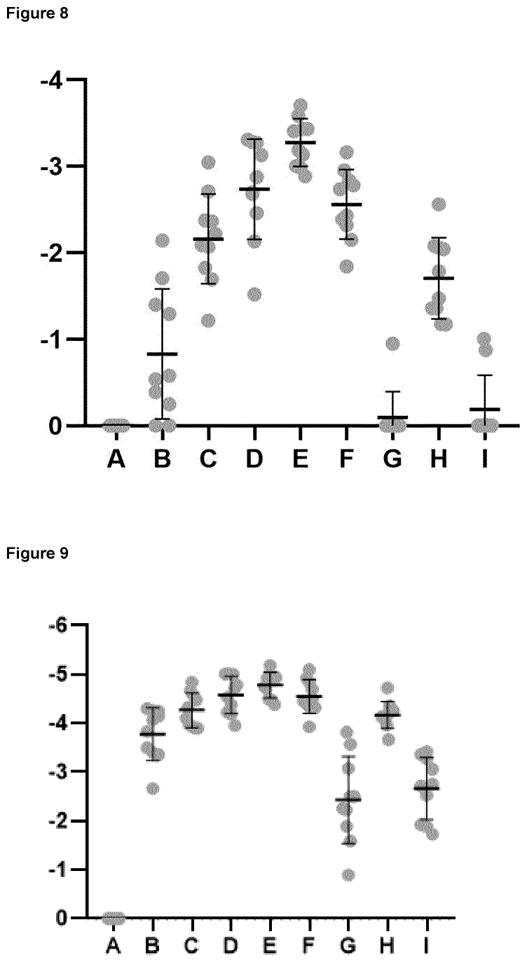

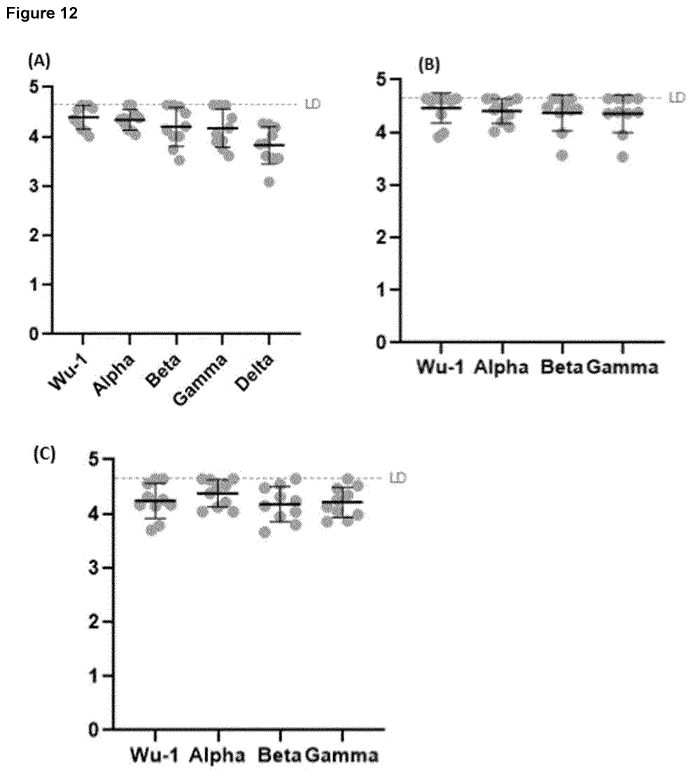

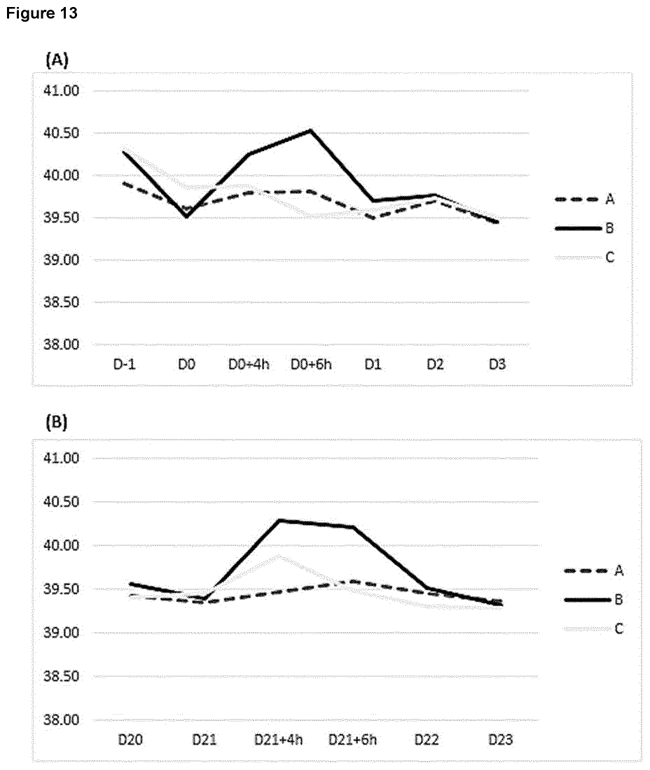

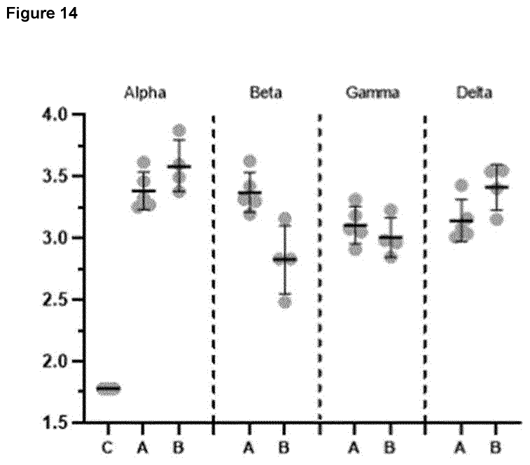

The inventors also designed a new dimeric RBD antigen by fusing two RBD monomers of two different SARS-CoV-2 variants (UK and South African variant), generating a vaccine candidate comprising fusion dimeric RBD antigens. The ability of the fusion dimeric RBD antigens to induce antibodies against SARS-CoV-2 virus in comparison with the non-fusion dimers of Wuhan variant was tested and the results are shown in FIG. 8. The results showed that, even when the animals received a low dose of the vaccine comprising fusion dimeric RBD antigens without any immunostimulant (Groups B and C), they were able to produce higher anti-SARS-CoV-2 RBD IgG antibody titers than groups that received a vaccine formulation comprising 20 pg dose of the non-fusion Wuhan RBD dimers with adjuvant alone (Group G) or with adjuvant together with QS-21 immunostimulant (Group I). Further, at equal doses of the antigen (20 pg), the fusion dimeric RBD antigens also elicited an increased response even if the composition did not comprise any immunostimulant (Group D) when compared to the groups that received non-fusion Wuhan RBD antigens plus adjuvant and MPLA immunostimulant (Group H). It is also shown herein that the immunization of animals with said novel recombinant fusion dimeric RBD SARS-CoV-2 antigen elicited pseudovirus-neutralizing antibody titres against different SARS-CoV-2 variants (Wuhan, U.K., South Africa, Indian and Brazil variants) in all the groups, which also demonstrates that the neutralizing antibody titters generated from vaccinating mice with the fusion dimeric RBD antigen are maintained at high levels regardless of the variant tested and the presence or absence of an immunostimulant such as of MPLA (Group E) or QS.21 (Group F) in the vaccine formulation (FIG. 12). Next, studies using pigs, which is an animal model closer to humans, were performed. The results showed that the fusion dimeric RBD antigen induced significant higher titres against the South Africa variant compared to the commercial vaccine, and that the vaccine comprising the fusion dimeric RBD antigen is also safer than the commercial mRNA based vaccine, as measured by temperature measurements in pigs after vaccination (see FIGS. 14 and 13). These results indicate that the fusion dimeric RBD antigen present a superior balance between safety and immunogenicity compared to commercially available vaccines, as it is able to induce neutralizing antibodies without increasing body temperature or causing adverse effects.

Further studies, particularly clinical trials in humans were also performed. The results showed that the fusion dimeric RBD antigen of the invention elicited high levels of neutralizing antibodies against different SARS-CoV-2 variants such as Beta, Delta and Omicron variants. Moreover, an increased and better immunogenicity response against SARS-CoV-2 variants of concern was also observed for the fusion dimeric RBD antigen of the invention when compared to a mRNA based vaccine (Comirnaty, BioNTech), see Example 11.

Altogether, these results unexpectedly showed the strong capacity to generate anti-SARS-CoV-2 RBD antibodies of formulations comprising fusion dimeric RBD antigens and of formulations comprising non-fusion RBD antigens with an increased proportion of dimeric RBD antigen over monomeric RBD antigen (more than 50%). Further, it is also shown the increased potential in immunogenicity and safety of the new recombinant fusion dimeric RBD antigen based on two different SARS-CoV-2 variants that was generated herein.

Thus, in a second aspect of the invention the protein subunit vaccine comprises at least one antigen characterized in that it comprises or consist of at least two monomers from at least one variant of severe acute respiratory syndrome coronavirus 2 (SARS-CoV-2), wherein each of the monomers are selected from the group consisting of the S1 subunit of the Spike protein or the receptor-binding domain (RBD) of the Spike protein, or any immunogenic fragments thereof, and wherein the two monomers are chemically bound to each other, optionally through a linker, forming a dimer, preferably a fusion dimer or a non-fusion dimer. It is noted that the terms “fusion dimer”, “non-fusion dimer”, and “bound to each other” are defined in the definitions section above. In a preferred embodiment, the two monomers are different. By “different monomers” is meant that each monomer of the dimer has different amino acid sequence, for example, a mixture of RBDs antigens derived from different variants, or a mixture of RBD and S1 antigens derived from the same or from different variants. Preferably, the amino acid sequence of each of the monomers in the fusion dimer corresponds to different SARS-CoV-2 amino acid sequences.

It is noted that the dimeric antigen defined above is much preferably formed by two monomers. Thus, in a preferred embodiment, the protein subunit vaccine comprises at least one antigen comprising or consisting of two monomers from at least one variant of severe acute respiratory syndrome coronavirus 2 (SARS-CoV-2), wherein each of said monomers are selected from the group consisting of the S1 subunit of the Spike protein or the receptor-binding domain (RBD) of the Spike protein, or any immunogenic fragments thereof, and wherein the two monomers are chemically bound to each other, optionally through a linker, forming a dimer.

In an embodiment the dimeric antigen is a homodimer characterized by comprising, consisting of or consisting essentially of two monomers, wherein each of the monomers are selected from the group consisting of the S1 subunit of the Spike protein or the receptor-binding monomer (RBD) of the Spike protein, or any immunogenic fragments thereof, of at least one variant of SARS-CoV-2. In an embodiment, the dimeric antigen is a homodimer that comprises consists of or consists essentially of two monomers of RBD of a selected SARS-CoV-2 variant. In another embodiment, the at least one antigen is a homodimer that comprises consists of or consists essentially of two monomers of the S1 subunit of a selected SARS-CoV-2 variant. In an embodiment, each monomer comprised in the homodimeric antigen is derived from the same SARS-CoV-2 variant. In a preferred embodiment, both monomers comprised in the dimeric antigen are the RBD of the Spike protein from at least one variant of SARS-CoV-2 virus.

In another embodiment, the monomers that form the dimeric antigen are different in their amino acid sequence (also called heterodimer). The differences in their amino acid sequence can be because the monomers are derived from different SARS-CoV-2 variants or because they are different antigens from a selected SARS-CoV-2 variant. In an embodiment, the at least one antigen is a heterodimer characterized by consisting of two monomers, wherein a first monomer is selected from the group consisting of the S1 subunit of the Spike protein or the receptor-binding domain (RBD) of the Spike protein, or any immunogenic fragments thereof of a first SARS-CoV-2 variant, and a second monomer is selected from the group consisting of the S1 subunit of the Spike protein or the receptor-binding domain (RBD) of the Spike protein, or any immunogenic fragments thereof of a second SARS-CoV-2 variant, wherein the first and the second SARS-CoV-2 variant are the same or are different. In another embodiment, the heterodimeric antigen consists of two monomers, wherein one is the S1 subunit and the other is the RBD.

In a preferred embodiment, the dimeric antigen comprises or consists of a first and a second monomer that are bound to each other. As defined above, “bound to each other” means that they are chemically connected one to each other by very weak, weak, strong, or very strong bonds. In a preferred embodiment, the dimeric antigen is a non-fusion dimer, wherein the two monomers of the non-fusion dimer are bound by reversible bonds, preferably disulfide bonds. In an embodiment, the two monomers of the non-fusion dimeric antigen are identical in amino acid sequence. In another embodiment, the two monomers of the non-fusion dimeric antigen are different in their amino acid sequence.

In another embodiment, the dimeric antigen is a fusion dimer comprising or consisting of two monomers, wherein the two monomers are part of a single polypeptide. In a preferred embodiment, the two monomers of the fusion dimeric antigen are part of a single polypeptide since they are connected by at least a peptide bond. In the case of the fusion dimeric antigen, the two monomers are synthetized as part of the same polypeptide chain by the same translation complex. Thus, the two monomers of the fusion dimeric antigen are comprised within the same molecule, this means forming one antigen. In an embodiment, the fusion dimer comprises at least two monomers that are located in tandem or in a tandem repeat, in any order, and are optionally connected by a linker peptide. In an embodiment, the two monomers of the fusion dimer are identical in amino acid sequence. In another embodiment, the two monomers of the fusion dimeric antigen are different in their amino acid sequence.

In another embodiment, the protein subunit vaccine according to the second aspect or any of its embodiments comprises a mixture of antigens that are present in different forms, such as monomers and dimers, wherein the dimers can be non-fusion and/or fusion, as defined above. In an embodiment, the protein subunit vaccine preferably comprises a mixture of monomeric antigens and dimeric antigens, wherein the dimers can be non-fusion and/or fusion, wherein the antigens comprise RBD monomers. In an embodiment, the protein subunit vaccine comprises higher proportion of dimeric antigens (non-fusion or fusion) than monomeric antigens. In an embodiment the antigen proportion comprised in the protein subunit vaccine is at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% non-fusion or fusion dimers comprising RBD monomers. The calculation of the percentage of monomeric antigen comprising one RBD monomer and dimeric antigens comprising two RBD monomers can be determined by using standard methods, such as Size Exclusion Chromatography (SEC) or High-Performance Liquid Chromatography (HPLC). The area under the peak in the Size Exclusion Chromatography of the identified dimeric and monomeric peak represents the relative amount of the RBD-monomer and RBD-dimer. Obtaining a particular proportion of the dimeric RBD antigen over monomeric RBD antigen is known by the skilled in the art, by mixing for example, different volumes of the dimeric RBD antigen and the monomeric RBD antigen. In an embodiment, the protein subunit vaccine comprises higher proportion of non-fusion dimeric RBD antigens than monomeric RBD antigens. In an embodiment, the protein subunit vaccine comprises a percentage of at least 35%, 40%, 45%, 50%, 51%, 55%, 60%, 65%, 70%, 75%, 80%, 85%, 90%, 95% of non-fusion dimeric RBD antigens. In a preferred embodiment, the protein subunit vaccine comprises a mixture of at least a monomeric RBD antigen and at least a dimeric RBD antigen, wherein at least 35%, 40%, 45%, 50%, 51%, 55%, 60%, 65%, 70%, 75%, 80%, 85%, 90%, 95% of the total antigen comprised in the protein subunit vaccine is a dimeric RBD antigen.

In an embodiment of the second aspect, each of the monomers comprised in the non-fusion or in the fusion dimeric antigens are derived from the same SARS-CoV-2 variant. In an embodiment of the second aspect, each of the monomers comprised in the non-fusion or in the fusion dimeric antigen are derived from the same SARS-CoV-2 variant, wherein the SARS-CoV-2 variant is selected from the variants of concern (VOC), as defined by the Centers for Disease Control and Prevention (CDC) “SARS-CoV-2 Variant Classifications and Definitions”. In an embodiment of the second aspect, each of the monomers comprised in the non-fusion or in the fusion dimeric antigen are derived from the same SARS-CoV-2 variant, wherein the variant is selected from the group including, but not limited, to Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) Linage B.1.1.7 (United Kingdom variant), Linage B.1.617.2 or G/478K.V1 (Delta Variant) or Linage B.1.1.529 or GR/484A (Omicron variant) or any combination thereof. In an embodiment of the second aspect, each of the monomers comprised in the non-fusion or in the fusion dimeric antigen are derived from the same SARS-CoV-2 variant, wherein the variant is selected from the group comprising or consisting of Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) Linage B.1.1.7 (United Kingdom variant), Linage B.1.617.2 or G/478K.V1 (Delta Variant) or Linage B.1.1.529 or GR/484A (Omicron variant) or any combination thereof.

In another embodiment, each of the two monomers comprised in the non-fusion or in the fusion dimeric antigen are derived from a different SARS-CoV-2 variant. In an embodiment, each of the monomers comprised in the non-fusion or in the fusion dimeric antigen are derived from a different SARS-CoV-2 variant, wherein each of the SARS-CoV-2 variant is selected from the variants of concern (VOC), as defined by the Centers for Disease Control and Prevention (CDC) “SARS-CoV-2 Variant Classifications and Definitions”. In an embodiment, each of the monomers comprised in the non-fusion or in the fusion dimeric antigen are derived from a different SARS-CoV-2 variant, wherein each of the SARS-CoV-2 variant is selected from the group including, but not limited to, Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) Linage B.1.1.7 (United Kingdom variant), Linage B.1.617.2 or G/478K.V1 (Delta Variant) or Linage B.1.1.529 or GR/484A (Omicron variant) or any combination thereof. In an embodiment, each of the monomers comprised in the non-fusion or in the fusion dimeric antigen are derived from a different SARS-CoV-2 variant, wherein each of the SARS-CoV-2 variant is selected from the group consisting of Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) Linage B.1.1.7 (United Kingdom variant), Linage B.1.617.2 or G/478K.V1 (Delta Variant) or Linage B.1.1.529 or GR/484A (Omicron variant) or any combination thereof. It is to be understood that any combination of the different SARS-CoV-2 variants in each of the monomers of the non-fusion or in the fusion dimeric antigen is comprised within the scope of the present invention.

In an embodiment of the second aspect, one or both of the two monomers of the non-fusion and the fusion dimeric antigen are the receptor-binding domain (RBD) of the Spike protein from at least one variant of severe acute respiratory syndrome coronavirus 2 (SARS-CoV-2). In a preferred embodiment, one or both of the two monomers of the non-fusion and the fusion dimeric antigen are the receptor-binding domain (RBD) of the Spike protein that comprises, consists, or consists essentially of amino acid residues 319 to 537 of the SARS-CoV-2. In a preferred embodiment, one or both of the two monomers non-fusion and the fusion dimeric antigen are the receptor-binding domain (RBD) of the Spike protein that comprises, consists, or consists essentially of amino acid residues 319 to 541 of the SARS-CoV-2. In a preferred embodiment, both of the two monomers of the non-fusion and the fusion dimeric antigen are the receptor-binding domain (RBD) of the Spike protein from at least one variant of severe acute respiratory syndrome coronavirus 2 (SARS-CoV-2), wherein said RBD monomers has/have at least 60%, 70%, 80%, 85%, 90%, 91%, 92%, 93%, 94%, 95%, 96%, 97%, 98%, 99%, 100% sequence identity over its full length with any of SEQ ID NO 1, SEQ ID NO 3, or SEQ ID NO 4, or any combination thereof.

In a preferred embodiment, the antigen is a non-fusion or a fusion dimer comprising two RBD monomers from at least one variant of SARS-CoV-2, wherein the at least one variant of SARS-CoV-2 is selected from the variants of concern (VOC).

In a preferred embodiment, the antigen is a non-fusion or fusion dimer comprising two RBD monomers from at least one variant of SARS-CoV-2 wherein the variant is selected from the group including, but not limited to Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) Linage B.1.1.7 (United Kingdom variant), Linage B.1.617.2 or G/478K.V1 (Delta Variant) or Linage B.1.1.529 or GR/484A (Omicron variant) or any combination thereof.

In a preferred embodiment, the antigen is a non-fusion or a fusion dimer comprising two RBD monomers from at least one variant of SARS-CoV-2, wherein the at least one variant of SARS-CoV-2 is selected from the group consisting of Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) Linage B.1.1.7 (United Kingdom variant), Linage B.1.617.2 or G/478K.V1 (Delta Variant) or Linage B.1.1.529 or GR/484A (Omicron variant) or any combination thereof.

In an embodiment, the protein subunit vaccine comprises or consists of at least one non-fusion dimer, and the non-fusion dimer comprises or consists of a first monomer and a second monomer, both derived from the Wuhan-Hu-1 seafood market pneumonia virus isolate (GenBank accession number: MN908947), and wherein the two monomers of the non-fusion dimer are bound by reversible bonds. In another embodiment, the first and/or the second monomers of the non-fusion dimer comprises, consists, or consists essentially of a protein that has at least 60%, 70%, 80%, 85%, 90%, 91%, 92%, 93%, 94%, 95%, 96%, 97%, 98%, 99%, 100% sequence identity over its full length with any of SEQ ID NO 1, SEQ ID NO 3, or SEQ ID NO 4, or any combination thereof.

In an embodiment of the second aspect, the fusion dimer consists of a first RBD monomer from a first SARS-CoV-2 variant and a second RBD monomer from a different second SARS-CoV-2 variant. Preferably, the protein subunit vaccine comprises at least one antigen, wherein the at least one antigen is a fusion dimer, and wherein the fusion dimer comprises, consists, or consists essentially of a first monomer derived from the Linage B.1.351 (South African SARS-CoV-2 variant), and a second monomer derived from the Linage B.1.1.7 (United Kingdom SARS-CoV-2 variant), and wherein the two monomers of the fusion dimer are part of a single polypeptide. Preferably, the fusion dimer comprises two RBD monomers (herein after referred to as fusion dimeric RBD antigen).

In an embodiment, the fusion dimeric RBD antigen comprises a first monomer derived from the B.1.351 variant and a second monomer derived from the B.1.1.7 variant. More preferably, the fusion dimeric RBD antigen comprises a first RBD monomer that comprises, consists of or consists essentially of SEQ ID NO: 4 or a sequence with at least 50%, 60%, 70%, 80%, 85%, 90%, 91%, 92%, 93%, 94%, 95%, 96%, 97%, 98%, 99% or 100% sequence identity over its full length with SEQ ID NO: 4, and a second RBD monomer that comprises, consists of or consists essentially of SEQ ID NO: 3 or a sequence with at least 50%, 60%, 70%, 80%, 85%, 90%, 91%, 92%, 93%, 94%, 95%, 96%, 97%, 98%, 99% or 100% sequence identity over its full length with SEQ ID NO: 3. In some embodiments, the fusion dimeric RBD antigen comprises a first RBD monomer that comprises, consists, or consists essentially of SEQ ID NO: 4 and a second RBD monomer that comprises, consists, or consists essentially of SEQ ID NO: 3. More preferably, the fusion dimeric RBD antigen comprises, consists, or consists essentially of a protein that has at least 60%, 70%, 80%, 85%, 90%, 91%, 92%, 93%, 94%, 95%, 96%, 97%, 98%, 99%, 100% sequence identity over its full length with SEQ ID NO: 5. In some embodiments, the fusion dimeric RBD antigen comprises, consists, or consists essentially of SEQ ID NO: 5 (fusion dimeric RBD antigen sequence).

In another embodiment, the fusion dimeric RBD antigen is encoded by a nucleotide sequence having at least 50%, 60%, 70%, 80%, 85%, 90%, 91%, 92%, 93%, 94%, 95%, 96%, 97%, 98%, 99% or 100% sequence identity over its full length of SEQ ID NO 7. In some embodiments, the fusion dimeric RBD antigen is encoded by a nucleotide sequence that comprises, consists, or consists essentially of SEQ ID NO: 7 (fusion dimeric RBD nucleotide sequence). In another embodiment, the fusion dimeric RBD antigen is encoded by a nucleotide sequence having at least 50%, 60%, 70%, 80%, 85%, 90%, 91%, 92%, 93%, 94%, 95%, 96%, 97%, 98%, 99% or 100% sequence identity over its full length of SEQ ID NO: 8. In some embodiments, the fusion dimeric RBD antigen is encoded by a nucleotide sequence that comprises, consists, or consists essentially of SEQ ID NO: 8 (fusion dimeric RBD nucleotide sequence).

In an embodiment, the protein subunit vaccine, preferably the fusion dimeric RBD antigen, is capable of inducing an immunogenic and/or protective immune response without increasing or modifying the basal body temperature of the subject immunized with the vaccine, being an increase in the basal body temperature understood as an increase of 0.5° C., 0.6° C., 0.7° C., 0.8° C., 0.9° C., 1° C., 1.2° C., 1.4° C., 1.6° C., 1.8° C., 2° C. or more than 2° C. the body temperature after immunization. In an embodiment, the protein subunit vaccine, preferably the fusion dimeric RBD antigen, is capable of inducing an immunogenic and/or protective immune response without producing significant adverse effects. Preferably, the protein subunit vaccine, preferably the fusion dimeric RBD antigen, is capable of inducing an immunogenic and/or protective immune response without producing significant adverse effects such as fatigue, pain at the site of injection, or tenderness, as shown in Example 11.

In an embodiment, any of the monomers comprised in the antigens of the first or second aspects of the invention or from any of its embodiments can be selected from the group consisting of the S1 subunit of the Spike protein or the receptor-binding monomer (RBD) of the Spike protein, or any immunogenic fragments thereof, comprising in its amino acid sequence a tag sequence or a signal peptide sequence, or both. In an embodiment, the RBD monomer comprises a signal peptide sequence at the N-terminus. In an embodiment, the signal peptide is located at the N-terminus and is selected from the group that consists of SEQ ID NO: 6 or SEQ ID NO: 10. In an alternative embodiment, the signal peptide may be replaced with any signal peptide that enables the expression of the at least one antigen. In an alternative embodiment, the processed antigen does not comprise the signal peptide. Following the expression of the at least one monomer the N-terminal signal peptide is cleaved. In an embodiment, the monomer comprises a tag sequence, preferably His tag sequence. The monomers and antigens described herein may also include additional modifications to the native sequence, such as additional internal deletions, additions and substitutions. These modifications may be deliberate, as through site-directed mutagenesis, or may be accidental, such as through naturally occurring mutational events.

In another embodiment, any of the antigens of the first or second aspects of the invention or from any of its embodiments is a recombinant expression product. Methods for producing recombinant antigens are known in the art, and they generally include cloning at least one antigen into an expression vector, preferably a plasmid, transfecting eukaryotic or prokaryotic cells with said plasmid vector, expressing said antigens in said cells and purifying the at least one antigen from the cells or from their supernatant.

In an embodiment, the plasmid vector is a mammalian expression vector. More preferably, the expression vector backbone used to express the at least one antigen is selected from the group consisting of the pcDNA3.4 (GENSCRIPT) or the pD2610-v10 (ATUM). In a preferred embodiment, the DNA sequence for the expression of the antigens of the invention is codon-optimized and inserted into the vector selected from the group consisting of pcDNA3.4 or the vector pD2610-v10 (ATUM). In an embodiment, the cells used to express the at least one antigen are eukaryotic cells, preferably CHO cells or HEK293 mammalian cells. In a preferred embodiment, the at least one antigen is collected and purified from the culture supernatant.

It is to be understood that the antigen or antigens comprised in the protein subunit vaccine provided herein are produced and maintained under suitable media conditions that allow the proper folding of said antigen or antigens. The skilled artisan would know the physical and chemical conditions to maintain and preserve the desired structure of the antigens, including in their monomeric and multimeric forms, during all the stages of production. In an embodiment, the media conditions are chosen so the dimeric form of the antigen is favoured over the monomeric form. Spontaneous dimerization can be governed by very weak, weak, strong, or very strong bonds and can be covalent bonds (e.g. disulfide bridges) or non-covalent bonds. The skilled artisan would know how to optimize the media conditions to obtain the desired proportion of dimeric and/or monomeric antigens. For example, the use of high temperatures (above the melting temperature of the dimer) or ionic detergents (such as SDS) are not recommended for spontaneous dimer formation.

In an embodiment, the antigens are produced in the presence of oxidizing agents such as glutathione. In an embodiment, culture media where the antigens are being produced in the absence of reducing agents such as dithiothreitol. In an embodiment, the antigen or antigens of the protein subunit vaccine are produced at a temperature suitable to preserve their structure or to favour the formation of dimers. A skilled in the art also knows to adjust said temperature. In an embodiment, the temperature of the production of the antigens range 30° C. to 40° C., preferably from 33° C. to 37° C., most preferably 33° C.

In an embodiment, the pH of the protein subunit vaccine and/or the media where the antigens are produced is kept at pH 7 or below. In an embodiment, the pH of the protein subunit vaccine and/or the media where the antigens are produced is kept at pH 7 or above. In an embodiment, the pH is acidic pH (below 7). In an embodiment, the pH is basic pH (above 7). In an embodiment, the pH is neutral (about 7). In an embodiment, the pH of the protein subunit vaccine is about 0, 1, 2, 3, 4, 5, 6, 7, 8, 9, 10, 11, 12, 13, or 14. In a preferred embodiment, the pH of the protein subunit vaccine ranges from 4 to 9, from 5 to 8, from 5 to 7.5, from 5 to 7, from 5 to 6.5, from 5.5 to 6.5, or any value comprised within these ranges. In an embodiment, the pH ranges from 5 to 9, from 5.5 to 9, from 6 to 9, from 6.5 to 9, from 7 to 9, from 7 to 8, or any value comprised within these ranges.

The selection of a proper adjuvant that helps promoting an appropriate immune response against a target pathogen at both innate and adaptative levels such that protective immunity can be elicited while maintaining the safety profile is critical and not straightforward. The selection of the wrong adjuvant may render a particular vaccine antigen inadequate. Thus, vaccine antigen selection must carefully consider which adjuvant or combination of adjuvants and/or immunostimulants are used to avoid discarding potentially effective vaccine antigen candidates and to help with vaccine development. The present invention disclosed herein also shows that squalene or squalene oil-in-water adjuvant formulations are suitable adjuvants to be included in protein subunit SARS-CoV-2 vaccines, particularly in vaccines comprising at least one antigen selected from the group consisting of the S1 subunit of the Spike protein or the receptor-binding domain (RBD) of the Spike protein. In particular, we herein show that protein subunit vaccines comprising at least one antigen characterized in that it comprises the S1 subunit or RBD monomers that are adjuvanted with squalene or squalene oil-in-water adjuvant formulation were able to elicit high neutralizing antibody titters against SARS-CoV-2 virus, as shown in FIG. 1, and to produce the release of cytokines which is indicative of the presence of a cellular immune response, as shown in FIG. 2. Further, the combination of the S1 subunit or RBD monomers with squalene or squalene oil-in-water adjuvant formulation and an immunostimulant, such as MPLA, also elicited high neutralizing antibody titters and an even higher cellular immune response (FIG. 2, groups C and G).

Thus, in a further embodiment of the first or second aspect, the protein subunit vaccine as defined above in the first aspect or second aspect or in any of its embodiments, further comprises at least one adjuvant, preferably MF59C.1. In a further embodiment, the at least one adjuvant is preferably a squalene or squalene oil-in-water adjuvant formulation. Further, in another embodiment, the protein subunit vaccines as defined above in the first or second aspect or any of its embodiments, further comprises at least one immunostimulant. The possible adjuvants and immunostimulants are defined below.

### The at Least One Adjuvant

As stated above, the protein subunit vaccine according to the first or the second aspect may further comprise at least one adjuvant. The at least one adjuvant may include, but is not limited to, aluminium salts (alum), such as aluminium hydroxide, aluminium phosphate, aluminium sulphate, etc., formulations of oil-in-water or water-in-oil emulsions such as complete Freund's Adjuvant (CFA) as well as the incomplete Freund's Adjuvant (IFA), mineral adjuvants, block copolymers, adjuvants formed by components of bacterial cell wall such as adjuvants including liposaccharides (e.g., lipid A or Monophosphoryl Lipid A (MPLA)), trehalose dimycolate (TDB), and components of the cell wall skeleton (CWS), heat shock proteins or the derivatives thereof, adjuvants derived from ADP-ribosylating bacterial toxins, which include diphtheria toxin (DT), pertussis toxin (PT), cholera toxin (CT), Escherichia coli heat-labile toxins (LT1 and LT2), Pseudomonas Endotoxin A and exotoxin, Bacillus cereus exoenzyme B, Bacillus sphaericus toxin, Clostridium botulinum toxins C2 and C3, Clostridium limosum exoenzyme as well as the toxins of Clostridium perfringens, Clostridium spiriforma and Clostridium difficile, Staphylococcus aureus, EDIM and mutants of mutant toxins such as CRM-197, non-toxic mutants of diphtheria toxin, chemokines, and cytokines (e. g., interleukin-2, interleukin-7, interleukin-12, granulocyte-macrophage colony stimulating factor (GM-CSF), interferon-y, interleukin-1 (IL-1p), and IL-1 (3 peptide or Sclavo Peptide), cytokine-containing liposomes, triterpenoid glycosides or saponins (e. g., ISCOMs, QuilA and QS-21), squalene or squalene oil-in-water adjuvant formulation, squalane or squalane oil-in-water adjuvant formulations, such as SAF, MF59 and MF59C.1, MuramylDipeptide (MDP) derivatives, such as N-acetyl-muramyl-L-threonyl-D-isoglutamine (Threonyl-MDP), GMDP, N-acetyl-normuramyl-L-alanyl-D-isoglutamine, N-acetylmuramyl-L-alanyl-D-isoglutaminyl-L-alanine2-(1′-2′-dipalmitoyl-sn-glycero-3-hydroxyphosphoryloxy)-ethylamine, muramyl tripeptide phosphatidylethanolamine (MTP-PE), unmethylated CpG dinucleotides and oligonucleotides, such as bacterial DNA and fragments thereof, oligo deoxynucleotides (ODN), and polyphosphazenes.

Other suitable mineral adjuvants include, but are not limited to, aluminum hydroxide gel (ALHYDROGEL, REHYDRAGEL), aluminum phosphate gel (including aluminum hydroxyphosphate gel (AlPO₄; Adju-Phos CRODA)), calcium phosphate, N-acetyl-muramyl-L-threonyl-D-isoglutamine (thr-MDP)-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine.

In another embodiment, a microparticulate adjuvant is used. Microparticulate adjuvants include, but are not limited to biodegradable and biocompatible polyesters, homo- and copolymers of lactic acid (PLA) and glycolic acid (PGA), poly (lactide-co-glycolides) (PLGA) microparticles, polymers that self-associate into particulates (poloxamer particles), soluble polymers (polyphosphazenes), and virus-like particles (VLPs) such as recombinant protein particulates, e. g., hepatitis B surface antigen (HbsAg).

Yet another type of adjuvants that may be used include mucosal adjuvants, including but not limited to heat-labile enterotoxin from Escherichia coli (LT), cholera holotoxin (CT) and cholera Toxin B Subunit (CTB) from Vibrio cholera, mutant toxins (e. g. LTK63 and LTR72), microparticles, and polymerized liposomes. Additional examples of mucous targeting adjuvants are E. coli mutant heat-labile toxin LT's with reduced toxicity, live attenuated organisms that bind M cells of the gastrointestinal tract, such as V. cholera and Salmonella typhi, Mycobacterium bovis (BCG), in addition to mucosal targeted particulate carriers such as phospholipid artificial membrane vesicles, copolymer microspheres, lipophilic immune-stimulating complexes and bacterial outer membrane protein preparations (proteosomes).

Other adjuvants known in the art are also included within the scope of the invention (see, e.g., Vaccine Design: The Subunit and Adjuvant Approach, Chap. 7, Michael F.).

Preferably, the at least one adjuvant is selected from the list consisting of aluminum phosphate gel adjuvant, preferably AlPO₄ gel; Adju-Phos CRODA, or squalene or squalene oil-in-water adjuvant formulations, preferably MF59C.1 or derivatives thereof. More preferably, the at least one adjuvant is MF59C.1. More preferably, the at least one adjuvant is a squalene or squalene oil-in-water adjuvant formulation.

MF59 adjuvants are oil-in-water emulsions composed of squalene (2, 6, 10, 15, 23-hexamethyl-2, 6, 10, 14, 18, 22-tetracosahexane) (4.3%), and two non-ionic surfactants, polysorbate 80 (also known as Tween 80) (0.5%) and sorbitan trioleate 85 (also known as Span 85) (0.5%). The emulsion is a milky-white oil-in-water emulsion which is stabilised by the two non-ionic surfactants (polysorbate 80 and sorbitan trioleate). The fundamental process involves dispersing sorbitan trioleate in squalene and polysorbate 80 in aqueous buffer before high-speed mixing to form a coarse emulsion. The coarse emulsion is then passed repeatedly through a microfluidizer to produce an o/w emulsion of uniform small droplet size which can be sterile filtered and filled into vials for later use. The process is largely described in the art, for example in O'Hagan D.T. et al., The history of MF59(*) adjuvant: a phoenix that arose from the ashes. Expert Rev Vaccines. 2013 January; 12(1):13-30. MF59C.1 adjuvant is an optimized version of the MF59 original adjuvant which is composed exactly with the same components and further comprising a citrate buffer (citric acid, monohydrate and sodium citrate, dihydrate) in water for injection in order to provide an increased stability to the original MF59 adjuvant.

Methods to prepare the MF59C.1 adjuvant are also known by the skilled artisan (see O'Hagan D.T. et al., supra or in U.S. App. No. 2009/0208523).

In an embodiment, the adjuvant can be formulated as emulsions, oil-in-water formulations, together with copolymers, virosomes, liposomes, cochleated, or with immunostimulants. In an embodiment, the at least one adjuvant can be mixed (before or simultaneously upon administration) with the other components of the protein subunit vaccine or alternatively the at least one adjuvant is not mixed with the other components of the protein subunit vaccines but is separately co-administered with them. In a preferred embodiment, the adjuvant MF59C.1 is mixed with the at least one antigen. In a preferred embodiment, the adjuvant is a squalene or squalene oil-in-water adjuvant formulation and it is mixed with the at least one antigen.

Preferably, in the context of the present invention, when referring to “squalene or squalene oil-in-water adjuvant formulation/s” specific reference is made to oil-in-water emulsions composed of squalene (2, 6, 10, 15, 23-hexamethyl-2, 6, 10, 14, 18, 22-tetracosahexane) (4.3%), and two non-ionic surfactants, polysorbate 80 (also known as Tween 80) (0.5%) and sorbitan trioleate 85 (also known as Span 85) (0.5%). Said emulsion is a milky-white oil-in-water emulsion which is stabilised by the two non-ionic surfactants (polysorbate 80 and sorbitan trioleate). Preferably, the process involves dispersing sorbitan trioleate in squalene and polysorbate 80 in aqueous buffer before high-speed mixing to form a coarse emulsion. The coarse emulsion is then passed repeatedly through a microfluidizer to produce an o/w emulsion of uniform small droplet size which can be sterile filtered and filled into vials for later use. More preferably, it is noted that, in the context of the present invention, the following squalene or squalene oil-in-water adjuvant formulations are especially preferred and are selected from the following list, from herein after referred to as “specific squalene or squalene oil-in-water adjuvant formulations”:
Preferably, the specific squalene or squalene oil-in-water adjuvant formulation comprises or preferably consists of about 1 to 15 mg of squalene per dose, 0.1 to 2 mg of polysorbate 80 per dose, 0.1 to 2 mg of sorbitan trioleate per dose, 0.08 to 1 mg of sodium citrate per dose and 0.004 to 0.05 of citric acid per dose.
Preferably, the specific squalene or squalene oil-in-water adjuvant formulation comprises or preferably consists of about 1.46 mg of squalene, 0.18 mg of polysorbate 80, 0.18 mg of sorbitan trioleate, 0.099 mg of sodium citrate and 0.006 mg of citric acid per dose of 0.1 ml.
Preferably, the specific squalene or squalene oil-in-water adjuvant formulation comprises or preferably consists of 1.95 mg of squalene, 0.235 mg of polysorbate 80, 0.235 mg of sorbitan trioleate, 0.132 mg of sodium citrate and 0.008 mg of citric acid per dose of 0.1 ml.
Preferably, the specific squalene or squalene oil-in-water adjuvant formulation comprises or preferably consists of about 9.75 mg of squalene, 1.175 mg of polysorbate 80, 1.175 mg of sorbitan trioleate, 0.66 mg of sodium citrate and 0.04 mg of citric acid per dose of 0.5 ml.
Preferably, the specific squalene or squalene oil-in-water adjuvant formulation comprises or preferably consists of 10 to 60 mg/ml of squalene, 1 to 6 mg/ml of polysorbate 80, 1 to 6 mg/ml of sorbitan trioleate, 0.5 to 6 mg/ml of sodium citrate, and 0.01 to 0.5 mg/ml of citric acid.
Preferably, the specific squalene or squalene oil-in-water adjuvant formulation comprises or preferably consists of about 19.5 mg/ml of squalene, 2.35 mg/ml of polysorbate 80, 2.35 mg/ml of sorbitan trioleate, 1.32 mg/ml of sodium citrate, and 0.08 mg/ml of citric acid.
Preferably, the specific squalene or squalene oil-in-water adjuvant formulation comprises or preferably consists of about 39 mg/ml of squalene, 4.7 mg/ml of polysorbate 80, 4.7 mg/ml of sorbitan trioleate, 2.64 mg/ml of sodium citrate, and 0.16 mg/ml of citric acid.

In an embodiment, the MF59C.1 is formulated as about 1 to 15 mg of squalene per dose, 0.1 to 2 mg of polysorbate 80 per dose, 0.1 to 2 mg of sorbitan trioleate per dose, 0.08 to 1 mg of sodium citrate per dose and 0.004 to 0.05 of citric acid per dose. In an embodiment, the MF59C.1 is formulated as about 1.46 mg of squalene, 0.18 mg of polysorbate 80, 0.18 mg of sorbitan trioleate, 0.099 mg of sodium citrate and 0.006 mg of citric acid per dose of 0.1 ml. In an embodiment, the MF59C.1 is formulated as about 1.95 mg of squalene, 0.235 mg of polysorbate 80, 0.235 mg of sorbitan trioleate, 0.132 mg of sodium citrate and 0.008 mg of citric acid per dose of 0.1 ml. In a preferred embodiment, the MF59C.1 is formulated as about 9.75 mg of squalene, 1.175 mg of polysorbate 80, 1.175 mg of sorbitan trioleate, 0.66 mg of sodium citrate and 0.04 mg of citric acid per dose of 0.5 ml.

In an embodiment, the MF59C.1 is formulated as about 10 to 60 mg/ml of squalene, 1 to 6 mg/ml of polysorbate 80, 1 to 6 mg/ml of sorbitan trioleate, 0.5 to 6 mg/ml of sodium citrate, and 0.01 to 0.5 mg/ml of citric acid. In an embodiment, the MF59C.1 is formulated as about 19.5 mg/ml of squalene, 2.35 mg/ml of polysorbate 80, 2.35 mg/ml of sorbitan trioleate, 1.32 mg/ml of sodium citrate, and 0.08 mg/ml of citric acid. In a preferred embodiment, the MF59C.1 is formulated as about 39 mg/ml of squalene, 4.7 mg/ml of polysorbate 80, 4.7 mg/ml of sorbitan trioleate, 2.64 mg/ml of sodium citrate, and 0.16 mg/ml of citric acid.

In other embodiments, any of the above classes of adjuvants may be used in combination with each other or with other adjuvants, antigens, or immunostimulants.

### The at Least One Immunostimulant.

As stated above, the protein subunit vaccine according to the first or the second aspect or any of its embodiments can optionally further comprise at least one immunostimulant. The at least one immunostimulant may include, but is not limited to, a toll-like receptor (TLR) agonist, a NOD-like receptor agonist, or a cytokine. The toll-like receptor is mostly expressed in immune cells to perform a key role in immune activity and known to increase maturation of a dendritic cell through stimulus of the active pharmaceutical ingredient. The toll-like receptor agonist may include a member selected from the group consisting of, for example, TLR-1 agonist, TLR-2 agonist, TLR-3 agonist, TLR-4 agonist, TLR-5 agonist, TLR-6 agonist, TLR-7 agonist, TLR-8 agonist and TLR-9 agonist, but not be limited thereto. The NOD-like receptor, as an intracellular sensor of pathogen-associated molecular patterns (PAMPs) entering into cells through phagocytosis or pores and damage-associated molecular pattern molecules (DAMPS) associated with cell stress, is a part of a pattern recognition receptor and plays an important role in an innate immune response. The NOD-like receptor agonist may include, for example, NLRA agonist, NLRB agonist, NLRC agonist or NLRP agonist, but not be limited thereto. The cytokine is a generic name of proteins secreted by immune cells and known to induce proliferation of macrophages and lymphocytes or promote its differentiation. The cytokine may include, for example, IL-1α, IL-113, IL-1, IL-2, IL-3, IL-4, IL-5, IL-6, IL-7, IL-8, IL-9, IL-10, IL-11, IL-12, IL-13, IL-15, IL-16, IL-17, IL-18, IL-19, IL-20, IL-21, IL-22, IL-23, IL-24, IL-25, GM-CSF, G-CSF, M-CSF, TNF-α, TNF-β, IFNα, or IFNβ. Immunostimulants included in the present invention may also be poly (L:CU), CpG, imiquimod, resiquimod, dSLIM, toll-like receptor agonists such as monophosphoryl lipid A (MPLA), flagellin, a plasmid DNA double-strand DNA, a single-strand DNA, saponins such as QS-21, and an interleukin cytokine, but not be limited thereto.

In a preferred embodiment, the at least one immunostimulant is selected from the group consisting of toll-like receptor agonists such as Monophosphoryl lipid A (MPLA) or saponins such as C₉₂O₄₆H₁₄₈ (QS-21). In a preferred embodiment, the protein subunit vaccine comprises at least one immunostimulant, wherein the immunostimulant is selected from the group consisting of Monophosphoryl lipid A (MPLA) and/or C₉₂O₄₆H₁₄₈ (QS-21).

QS-21 is an acylated 3,28-bisdesmodic triterpene glycoside with molecular formula C₉₂O₄₆H₁₄₈ and molecular weight 1990 Da. It was originally designated as a particular fraction on a complex RP-H PLC trace, specifically the active fraction 21 (RP-HPLC peak) of the tree Quillaja saponaria, as described by Kensil C. et al. Separation and characterization of saponins with adjuvant activity from Quillaja saponaria Molina cortex. J Immunol 1991; 146:431e7 and by Ragupathi et al. Natural and synthetic saponin adjuvant QS-21 for vaccines against cancer. Exp Rev Vaccin 2011; 10:463e70. QS-21 fraction exhibits exceptional immunostimulant and adjuvant properties for a range of antigens. It possesses an ability to augment clinically significant antibody and T-cell responses to vaccine antigens against a variety of infectious diseases, degenerative disorders and cancers.

Monophosphoryl lipid A (MPLA or MPL) is a known immunostimulant obtained from bacterial lipopolysaccharides, normally from the lipopolysaccharide of Salmonella Minnesota, for example, like the one commercially available by the company SIGMA under the designation “Lipid A, monophosphoryl from Salmonella Minnesota Re 595 (Re mutant)” (product L 6895). In the context of the present invention, monophosphoryl lipid A also includes the derivatives and synthetic analogues thereof which are also suitable as immunostimulants, such as the derivative 3-deacylated (3D-MPL or 3D-MPLA), for example the one commercially available by company SIGMA under the designation MPL™. Synthetic analogues of monophosphoryl lipid A can also be used, for example, those described in the patent application WO2008/153541-A1 or those commercially available by companies Avanti Polar Lipids (product PHAD™) or AdipoGen (product AG-CU1-0002).

Methods to prepare the immunostimulants are known by the skilled artisan.

In an embodiment, the at least one immunostimulant can be mixed (before or simultaneously upon administration) with the other components of the protein subunit vaccine or alternatively the at least one immunostimulant is not mixed with the other components of the protein subunit vaccines but is separately co-administered with them. In a preferred embodiment, the immunostimulant MPLA is mixed with the at least one antigen and with the at least one adjuvant. In a preferred embodiment, the immunostimulant QS-21 is mixed with the at least one antigen and with the at least one adjuvant.

In another embodiment, any of the above classes of immunostimulants may be used in combination with each other or with other adjuvants, antigens, or immunostimulants.

### Dosages

The dosage of each of the components of the protein subunit vaccines as defined above can be determined readily by the skilled artisan, for example, by identifying doses effective to elicit a prophylactic or therapeutic immune response, e.g., by measuring the serum titre of vaccine specific immunoglobulins or by measuring the inhibitory ratio of serum samples compared to a control that does not receive the component. Further, the skilled artisan would also be able to adapt the dose of each of the components of the protein subunit vaccine to the subject in which the protein subunit vaccine is administrated. For example, the dose tested in mice models may be extrapolated to humans by including the same dosage tested in mice models or multiplying by 2, 3, 4, 5, 6, 7, or 8 times the dosage tested in mice models. Preferably, the adjuvant and immunostimulant adjusted dose for humans is obtained by multiplying the dosage tested in mice models by 5.

In an embodiment, the protein subunit vaccine according to the first or the second aspect or any of its embodiments comprises a therapeutically effective amount of the at least one antigen or antigens, as needed. By “therapeutically effective amount” is meant an amount that induces an immunogenic and protective immunological response in the uninfected, infected or unexposed individual to whom the vaccine is administered. The “therapeutically effective amount” refers to an amount of an antigen sufficient to induce an immune response that reduces at least one symptom or clinical sign which is associated to a SARS-CoV-2 infection or associated disease. As used herein, the term “immunogenic and protective immune response”, “protective immunity” or “protective immune response” means that the vaccinated subject is able to prevent the infection or disease, prevent the development of symptoms or clinical signs of that infection or disease, delay the onset of an infection or disease or its symptoms or its clinical signs, or decrease the severity of a subsequently infection or disease or symptom or clinical signs. Such a response will generally result in the development in the subject of a secretory, cellular and/or antibody-mediated immune response to the vaccine. Cellular-mediated immune responses include CD4+T helper cell responses, cytotoxic T lymphocytes CD8+, cell antiviral responses and antiviral chemokine responses. Antibody-mediated immune responses include those measured by serologic assays (such as virus neutralization assays, assays for ADCC, ELISAs, immunoblot assays, among other known assays). Thus, a protective immunological response includes, but is not limited to, one or more of the following effects: the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell and/or cytotoxic T cell. Several methods known in the art can be used to study the protective immunity generated by a vaccine candidate in preclinical and clinical trials. For instance, protective immunity can be analysed at a preclinical level by calculating the percentage of survival of vaccinated animals after a lethal or sublethal dose of SARS-CoV-2 infection, identifying the development of symptoms indicative of disease (decrease in body weight, fever), or quantifying viral load in infected organs.

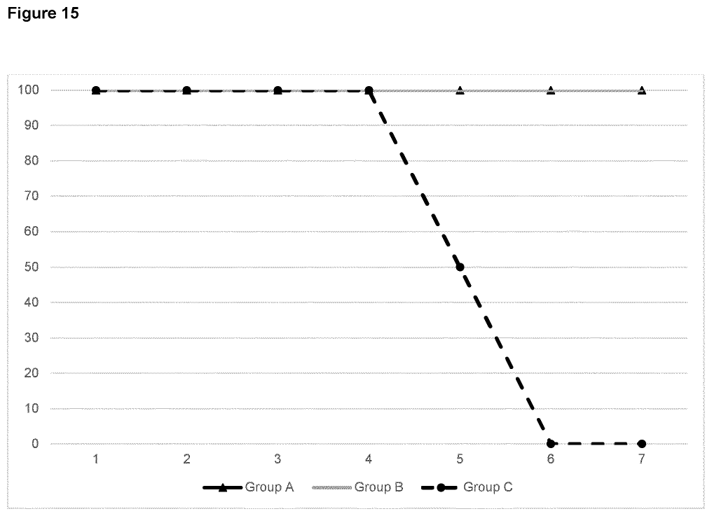

As shown in Example 10 and FIG. 15, a fully protective immune response (100% survival and no weight loss) was achieved with the vaccine comprising the fusion dimeric RBD variant SARS-CoV-2 antigen (a subunit vaccine comprising a fusion protein consisting of a first monomer comprising a RBD derived from the B.1. 351 (South Africa) variant and a second monomer comprising a RBD derived from the B.1.1.7 (UK) variant) in immunized mice that were challenged with a lethal dose of a Wuhan-like isolate comprising D614G mutation. Control group resulted in weight loss and 100% of mortality. Importantly, this full protection was achieved even at the lowest dose tested (10 pg) of the subunit vaccine.

Thus, in an embodiment, the subunit vaccine according to the first or the second aspect or any of their embodiments is able to prevent SARS-CoV-2 virus infection. “Preventing”, “to prevent” or “prevention”, include without limitation, decreasing, reducing or ameliorating the risk or the severity of a symptom, disorder, condition, or disease, and protecting an animal from a symptom, disorder, condition, or disease. In an embodiment, the subunit vaccine provided herein is applied or administered prophylactically and/or therapeutically. In a preferred embodiment, the subunit vaccine comprising at least one antigen, preferably comprising at least one fusion dimer, as defined in the first or the second aspects or any of their embodiment, is able to induce immunogenic and/or protective immune responses that are able to prevent SARS-CoV-2 virus infection and/or the clinical signs or manifestations associated to SARS-CoV-2 infection, caused from at least one or from any of the SARS-CoV-2 variants. By “clinical signs associated to SARS-CoV-2 infection” is included, but not limited to, symptoms such as fever, chills, fatigue, dry cough, loss of taste or smell, rash on skin, chest pain, body weight loss, anorexia, headache, myalgia, diarrhea, sputum production, sore throat, nasal congestion, dyspnea, rhinorrhea, lymphopenia and mortality. Preferably, the subunit vaccine provided herein is able to induce immunogenic and/or protective immune responses that are able to prevent SARS-CoV-2 virus infection and/or the clinical signs or manifestations associated to SARS-CoV-2 infection caused from at least one SARS-CoV-2 variant, wherein the variant is selected from the group including, but not limited, to Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) Linage B.1.1.7 (United Kingdom variant), Linage B.1.617.2 or G/478K.V1 (Delta Variant) or Linage B.1.1.529 or GR/484A (Omicron variant) or any combination thereof. More preferably, the fusion dimer that is able to induce immunogenic and/or protective immune responses that are able to prevent SARS-CoV-2 infection, and/or the clinical signs or manifestations associated to SARS-CoV-2 infection, caused from at least one or from any of the SARS-CoV-2 variants is the fusion dimeric RBD antigen that comprises, consists, or consists essentially of a protein that has at least 60%, 70%, 80%, 85%, 90%, 91%, 92%, 93%, 94%, 95%, 96%, 97%, 98%, 99%, 100% sequence identity over its full length with SEQ ID NO 5, or the fusion dimeric RBD antigen that is encoded by a nucleotide sequence having at least 50%, 60%, 70%, 80%, 85%, 90%, 91%, 92%, 93%, 94%, 95%, 96%, 97%, 98%, 99% or 100% sequence identity over its full length of SEQ ID NO 7.

In an embodiment, the subunit vaccine according to the first or the second aspect or any of their embodiments is able to prevent mortality and weight loss caused by SARS-CoV-2 infection. In a preferred embodiment, the subunit vaccine comprising at least one antigen, preferably comprising at least one fusion dimer, as defined in the first or the second aspects or any of their embodiment, is able to induce immunogenic and/or protective immune responses that are able to prevent mortality and weight loss caused by SARS-CoV-2 virus infection from at least one or from any of the SARS-CoV-2 variants. Preferably, the subunit vaccine provided herein is able to induce immunogenic and/or protective immune responses that are able to prevent mortality and weight loss caused by SARS-CoV-2 virus infection from at least one SARS-CoV-2 variant, wherein the variant is selected from the group including, but not limited, to Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) Linage B.1.1.7 (United Kingdom variant), Linage B.1.617.2 or G/478K.V1 (Delta Variant) or Linage B.1.1.529 or GR/484A (Omicron variant) or any combination thereof. More preferably, the fusion dimer that is able to induce immunogenic and/or protective immune responses that are able to prevent mortality and weight loss caused by SARS-CoV-2 virus infection from at least one or from any of the SARS-CoV-2 variants is the fusion dimeric RBD antigen that comprises, consists, or consists essentially of a protein that has at least 60%, 70%, 80%, 85%, 90%, 91%, 92%, 93%, 94%, 95%, 96%, 97%, 98%, 99%, 100% sequence identity over its full length with SEQ ID NO 5, or the fusion dimeric RBD antigen that is encoded by a nucleotide sequence having at least 50%, 60%, 70%, 80%, 85%, 90%, 91%, 92%, 93%, 94%, 95%, 96%, 97%, 98%, 99% or 100% sequence identity over its full length of SEQ ID NO 7.

In an embodiment, the subunit vaccine provided herein is able to induce immunogenic and/or protective immune responses that are able to prevent SARS-CoV-2 virus infection wherein such immunogenic and/or protective immune response can be homologous and/or heterologous immunogenic. By “homologous immunogenicity” and “homologous protective immune responses” is referred herein as the immunity or protective immunity developed to one pathogen or variant after the host has had exposure to identical pathogens or antigens. By “heterologous immunogenicity” and “heterologous protective immune responses” is referred herein as the immunity or protective immunity developed to one pathogen or variant after the host has had exposure to non-identical pathogens or antigens.

Example 10 demonstrates that the fusion dimeric RBD variant SARS-CoV-2 subunit vaccine comprising two monomers derived from ZA and UK variant, respectively, was able to induce an immunogenic and protective immune response against a heterologous challenge, i.e., against a SARS-CoV-2 variant that was not used in the vaccine (Wuhan-like variant comprising D614G mutation). This supports the notion that the subunit vaccine provided herein, preferably the dimeric subunit vaccine, more preferably the fusion dimer, is able to induce heterologous immunogenic and/or heterologous protective immune responses.

In an embodiment, an effective amount of the protein subunit vaccine according to the first or the second aspect or any of its embodiments sufficient to bring about treatment or prevention of disease symptoms is administrated. An appropriate effective amount can be readily determined by one of skill in the art according to the age, sex, weight, and other physical and/or metabolic conditions of the subject in need thereof. A “therapeutically effective amount” can fall in a relatively broad range that can be determined through routine trials.

More particular, the possible dosages of the different components of the protein subunit vaccine according to the first or the second aspect or any of its embodiments are detailed below: Regarding the dosage of the antigen or antigens, in an embodiment, the total amount of the antigens comprised in the protein subunit vaccine is of about 1 μg per dose, 2 μg per dose, 3 μg per dose, 4 μg per dose, 5 μg per dose, 6 μg per dose, 7 μg per dose, 8 μg per dose, 9 μg per dose, 10 μg per dose, 11 μg per dose, 12 μg per dose, 13 μg per dose, 14 μg per dose, pg per dose, 16 μg per dose, 17 μg per dose, 18 μg per dose, 19 μg per dose, 20 μg per dose, 25 μg per dose, 30 μg per dose, 35 μg per dose, 40 μg per dose, 45 μg per dose, 50 μg per dose, 60 μg per dose, 70 μg per dose, 80 μg per dose, 90 μg per dose, or more than 100 μg per dose. Preferably, the total amount of the antigen or antigens comprised in protein subunit vaccine according to the first or the second or second aspect or any of its embodiments is about 10 μg of total antigen, 15 μg of total antigen, 20 μg of total antigen, 25 μg of total antigen, 30 μg of total antigen, 35 μg of total antigen, 40 μg of total antigen, 45 μg of total antigen, 50 μg of total antigen, 55 μg of total antigen, 60 μg of total antigen, 70 μg of total antigen, 80 μg of total antigen, 90 μg of total antigen or 100 μg of total antigen. Preferably, the total amount of the antigen or antigens is between 5 to 50 μg per dose, most preferably of 10 μg per dose, 20 μg per dose or 40 μg of antigen per dose.

With regards to the dosage of the adjuvant, in a preferred embodiment, the MF59C.1 adjuvant is present in the protein subunit vaccine according to the first or the second aspect or any of its embodiments at a relative percentage of adjuvant/antigen (v/v) of about 10%/90%, 20%/80%, 30%/70%, 40%/60%, 50%/50%, 60%/40%, 70%/30%, 80%/20%, 90%/10% per dose. Preferably, the amount of adjuvant present in the protein subunit vaccine with respect to the amount of antigen or antigens (% adjuvant/% antigen/s) (v/v) is 60-40%/40-60%, preferably 75%/25%, more preferably 50%/50%.

In a further preferred embodiment, the adjuvant is a specific squalene or squalene oil-in-water adjuvant formulation and it is present in the protein subunit vaccine according to the first or the second aspect or any of its embodiments at a relative percentage of adjuvant/antigen (v/v) of about 10%/90%, 20%/80%, 30%/70%, 40%/60%, 50%/50%, 60%/40%, 70%/30%, 80%/20%, 90%/10% per dose. Preferably, the amount of said adjuvant present in the protein subunit vaccine with respect to the amount of antigen or antigens (% adjuvant/% antigen/s) (v/v) is 60-40%/40-60%, preferably 75%/25%, more preferably 50%/50%.

In another embodiment, the aluminum phosphate adjuvant, preferably AlPO₄ gel, is present in the protein subunit vaccine according to the first or the second aspect or any of its embodiments at a dose of at least 5 mg per dose, 10 mg per dose, 20 mg per dose, 30 mg per dose, 40 mg per dose, 50 mg per dose, 60 mg per dose, 70 mg per dose, 80 mg per dose, 90 mg per dose, 100 mg per dose, or more than 100 mg per dose. In an embodiment, the aluminum phosphate adjuvant, preferably AlPO₄ gel, is present in the protein subunit vaccine according to the first or the second aspect or any of its embodiments at a dose of about 1-10 mg/dose, 5-15 mg/dose, 5-20 mg/dose, 10-20 mg/dose, 20-30 mg/dose, 30-40 mg/dose, 40-50 mg/dose, 50-60 mg/dose, 60-70 mg/dose, or 70-80 mg/dose. Preferably, the aluminum phosphate adjuvant, more preferably AlPO₄ gel, is formulated at a dose from 10 to 60 mg/dose, preferably about 10 mg/dose or 50 mg/dose.

Regarding the immunostimulant, in an embodiment, the total amount of the at least one immunostimulant optionally comprised in the protein subunit vaccine according to the first or the second aspect or any of its embodiments is about 5 μg per dose, 10 μg per dose, 15 μg per dose, 20 μg per dose, 25 μg per dose, 30 μg per dose, 35 μg per dose, 40 μg per dose, pg per dose, 50 μg per dose, 60 μg per dose, 70 μg per dose, 80 μg per dose, 90 μg per dose, 100 μg per dose. In a preferred embodiment, the total amount of immunostimulant per dose ranges from 1 to 100 μg, 10 to 90 μg, 20 to 80 μg, 20 to 70 μg, preferably 5 to 60 μg, or any values comprised within these ranges. More preferably the total amount of immunostimulant is 10 μg or 50 μg. In another preferred embodiment, the immunostimulant is selected from the group consisting of MPLA or QS-21, which are present in an amount of about 1 μg per dose, 2 μg per dose, 3 μg per dose, 4 μg per dose, 5 μg per dose, 6 μg per dose, 7 μg per dose, 8 μg per dose, 9 μg per dose, 10 μg per dose, 15 μg per dose, 20 μg per dose, pg per dose, 30 μg per dose, 35 μg per dose, 40 μg per dose, 45 μg per dose, 50 μg per dose, 60 μg per dose, 70 μg per dose, 80 μg per dose, 90 μg per dose, 100 μg per dose. Preferably, the at least one immunostimulant is MPLA or QS-21 at a dose that ranges from 1 to 100 μg per dose, 5 to 60 μg per dose, 10 to 90 μg per dose, 20 to 80 μg per dose, 20 to 70 μg per dose, preferably 5 to 60 μg per dose, or any values comprised within these ranges. More preferably the total amount of MPLA or QS-21 is 10 μg per dose or 50 μg per dose.

In other embodiments, any of the above classes of immunostimulants may be used in combination with each other or with other adjuvants and antigens. In an embodiment, the protein subunit vaccine comprises at least two immunostimulants. Preferably, the protein subunit vaccine comprises two immunostimulants that are MPLA and QS-21 mixed together and administrated simultaneously or separated in different container but administered simultaneously or sequentially. In a preferred embodiment, the total amount per dose of MPLA and QS-21 is about 1 μg, preferably 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 60 μg, 70 μg, 80 μg, 90 μg, 100 μg, wherein the two immunostimulants are at a ratio of 1:1; 1:2, 1:3, 1:4, 1:5, 1:6, 1:7, 1:8, 1:9 or 1:10, in any order (i.e., independently if it is QS-21:MPLA or MPLA:QS-21). Preferably, the ratio between both immunostimulants is 1:1. In an embodiment, the total amount of each of them ranges from 5 to 30 μg per dose, preferably 5 to 25 μg per dose. More preferably, the total amount of each of them is 10 μg per dose of which 5 μg are from QS-21 and the other 5 μg are from MPLA. In another embodiment the total amount is 50 μg dose of which 25 μg are from QS-21 and the other 25 μg are from MPLA.

In the following paragraphs, we shall indicate a non-exhaustive list of further preferred combinations of the at least one antigen, at least one adjuvant and, optionally, at least one immunostimulant in accordance with the protein subunit vaccine of the first or the second aspect or any of its embodiments. From herein after, when referring to the terms “RBD antigen” it is understood that this term encompasses any “monomeric RBD antigens” or “dimeric RBD antigens”, including “non-fusion RBD antigens” and “fusion RBD antigens”. From herein after, when referring to the terms “S1 antigen” it is understood that this term encompasses any “monomeric S1 antigens” or “dimeric S1 antigens”, including “non-fusion S1 antigens” and “fusion S1 antigens”.

In an embodiment, the protein subunit vaccine according to the first aspect or any of its embodiments comprises or consists of at least an RBD antigen and at least one adjuvant, wherein the at least one adjuvant is MF59C.1. In a further embodiment, the protein subunit vaccine comprises or consists of at least a S1 subunit antigen and at least one adjuvant, wherein the at least one adjuvant is MF59C.1. In an embodiment, the protein subunit vaccine consists of at least an RBD antigen and MF59C.1 as adjuvant. In another embodiment, the protein subunit vaccine consists of at least a S1 subunit antigen and MF59C.1 as adjuvant.

In an embodiment, the protein subunit vaccine according to the first aspect or any of its embodiments comprises or consists of at least an RBD antigen and at least one adjuvant, wherein the at least one adjuvant is the specific squalene or squalene oil-in-water adjuvant formulation. In a further embodiment, the protein subunit vaccine comprises or consists of at least a S1 subunit antigen and at least one adjuvant, wherein the at least one adjuvant is the specific squalene or squalene oil-in-water adjuvant formulation. In an embodiment, the protein subunit vaccine consists of at least an RBD antigen and the specific squalene or squalene oil-in-water adjuvant formulation. In another embodiment, the protein subunit vaccine consists of at least a S1 subunit antigen and the specific squalene or squalene oil-in-water adjuvant formulation.

In an embodiment, the protein subunit vaccine according to the first aspect or any of its embodiments comprises or consists of at least an RBD antigen and at least one adjuvant, wherein the at least one adjuvant is AlPO₄ gel. In a further embodiment, the protein subunit vaccine comprises or consists of at least a S1 subunit antigen and at least one adjuvant, wherein the at least one adjuvant is AlPO₄ gel. In an embodiment, the protein subunit vaccine consists of at least an RBD antigen and AlPO₄ gel as adjuvant. In another embodiment, the protein subunit vaccine consists of at least a S1 subunit antigen and AlPO₄ gel as adjuvant.

In a preferred embodiment, the protein subunit vaccine according to the first aspect comprises or consists of between 5 to 50 μg per dose, preferably 10 μg per dose, 20 μg per dose or 40 μg per dose of S1 subunit of the Spike protein of at least one variant SARS-CoV-2, and
i) MF59C.1 as adjuvant at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant.

In a preferred embodiment, the protein subunit vaccine according to the first aspect comprises or consists of between 5 to 50 μg per dose, preferably 10 μg per dose, 20 μg per dose or 40 μg per dose of S1 subunit of the Spike protein of at least one variant SARS-CoV-2, and
i) the specific squalene or squalene oil-in-water adjuvant formulation at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant.

In a preferred embodiment, the protein subunit vaccine according to the first aspect or any of its embodiments comprises between 5 to 50 μg per dose, preferably 10 μg per dose, 20 μg per dose or 40 μg per dose of the receptor-binding domain (RBD) antigen of the Spike protein of at least one variant SARS-CoV-2, and
i) MF59C.1 as adjuvant at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant.

In a preferred embodiment, the protein subunit vaccine according to the first aspect or any of its embodiments comprises between 5 to 50 μg per dose, preferably 10 μg per dose, 20 μg per dose or 40 μg per dose of the receptor-binding domain (RBD) antigen of the Spike protein of at least one variant SARS-CoV-2, and
i) the specific squalene or squalene oil-in-water adjuvant formulation at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant.

In a preferred embodiment, the protein subunit vaccine according to the first aspect comprises or consists of between 5 to 50 μg per dose, preferably 10 μg per dose, 20 μg per dose or 40 μg per dose of S1 subunit of the Spike protein of at least one variant SARS-CoV-2, and
i) an adjuvant comprising 10 to 60 mg/ml of squalene per dose, 1 to 6 mg/ml of polysorbate 80 per dose, 1 to 6 mg/ml of sorbitan trioleate per dose, 0.5 to 6 mg/ml of sodium citrate per dose, and 0.01 to 0.5 mg/ml of citric acid per dose, or
ii) 10-60 mg per dose, preferably 10 mg per dose or 50 mg per dose of AlPO₄ gel as adjuvant.

In a preferred embodiment, the protein subunit vaccine according to the first aspect or any of its embodiments comprises between 5 to 50 μg per dose, preferably 10 μg per dose, 20 μg per dose or 40 μg per dose of the receptor-binding domain (RBD) antigen of the Spike protein of at least one variant SARS-CoV-2, and
i) an adjuvant comprising 10 to 60 mg/ml of squalene per dose, 1 to 6 mg/ml of polysorbate 80 per dose, 1 to 6 mg/ml of sorbitan trioleate per dose, 0.5 to 6 mg/ml of sodium citrate per dose, and 0.01 to 0.5 mg/ml of citric acid per dose, or
ii) 10-60 mg per dose, preferably 10 mg per dose or 50 mg per dose of AlPO₄ gel as adjuvant.

According to the first aspect or any of its embodiments, the at least one immunostimulant can be combined with the at least one adjuvant as described above. In a preferred embodiment of the first aspect or any of its embodiments, the protein subunit vaccine comprises or consists of MF59C.1 as adjuvant and MPLA as immunostimulant. In a preferred embodiment, the protein subunit vaccine comprises or consists of at least one RBD antigen, MF59C.1, and MPLA. In another preferred embodiment, the protein subunit vaccine comprises or consists of at least one S1 subunit antigen, MF59C.1, and MPLA. In a preferred embodiment, the protein subunit vaccine comprises or consists of at least one RBD antigen, MF59C.1, and QS-21. In another preferred embodiment, the protein subunit vaccine comprises or consists of at least one S1 subunit antigen, MF59C.1, and QS-21.

According to the first aspect or any of its embodiments, the at least one immunostimulant can be combined with the at least one adjuvant as described above. In a preferred embodiment of the first aspect or any of its embodiments, the protein subunit vaccine comprises or consists of the specific squalene or squalene oil-in-water adjuvant formulation and MPLA as immunostimulant. In a preferred embodiment, the protein subunit vaccine comprises or consists of at least one RBD antigen, the specific squalene or squalene oil-in-water adjuvant formulation, and MPLA. In another preferred embodiment, the protein subunit vaccine comprises or consists of at least one S1 subunit antigen, the specific squalene or squalene oil-in-water adjuvant formulation, and MPLA. In a preferred embodiment, the protein subunit vaccine comprises or consists of at least one RBD antigen, the specific squalene or squalene oil-in-water adjuvant formulation, and QS-21. In another preferred embodiment, the protein subunit vaccine comprises or consists of at least one S1 subunit antigen, the specific squalene or squalene oil-in-water adjuvant formulation, and QS-21.

In a preferred embodiment of the first aspect or any of its embodiments, the protein subunit vaccine comprises or consists of AlPO₄ gel as adjuvant and MPLA as immunostimulant. In a preferred embodiment, the protein subunit vaccine comprises or consists of at least one RBD antigen, AlPO₄ gel, and MPLA. In another preferred embodiment, the protein subunit vaccine comprises or consists of at least one S1 subunit antigen, AlPO₄ gel, and MPLA. In a preferred embodiment, the protein subunit vaccine comprises or consists of at least one RBD antigen, AlPO₄ gel, and QS-21. In another preferred embodiment, the protein subunit vaccine comprises or consists of at least one S1 subunit antigen, AlPO₄ gel, and QS-21.

In a preferred embodiment of the first aspect or any of its embodiments, the protein subunit vaccine comprises or consists of at least one RBD antigen, MF59C.1, MPLA and QS-21. In another preferred embodiment, the protein subunit vaccine comprises or consists of at least one S1 subunit antigen, MF59C.1, and MPLA and QS-21. In a preferred embodiment, the protein subunit vaccine comprises or consists of at least one RBD antigen, AlPO₄ gel, MPLA and QS-21. In another preferred embodiment, the protein subunit vaccine comprises or consists of at least one S1 subunit antigen, AlPO₄ gel, and MPLA and QS-21.

In a preferred embodiment of the first aspect or any of its embodiments, the protein subunit vaccine comprises or consists of at least one RBD antigen, the specific squalene or squalene oil-in-water adjuvant formulation, MPLA and QS-21. In another preferred embodiment, the protein subunit vaccine comprises or consists of at least one S1 subunit antigen, the specific squalene or squalene oil-in-water adjuvant formulation, and MPLA and QS-21. In a preferred embodiment, the protein subunit vaccine comprises or consists of at least one RBD antigen, AlPO₄ gel, MPLA and QS-21. In another preferred embodiment, the protein subunit vaccine comprises or consists of at least one S1 subunit antigen, AlPO₄ gel, and MPLA and QS-21.

In a preferred embodiment of the first aspect or any of its embodiments, the protein subunit vaccine comprises between 5 to 50 μg per dose, preferably 10 μg per dose, 20 μg per dose or 40 μg per dose of S1 subunit of the Spike protein of at least one variant SARS-CoV-2, and
i) MF59C.1 as adjuvant at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the first aspect or any of its embodiments, the protein subunit vaccine comprises between 5 to 50 μg per dose, preferably 10 μg per dose, 20 μg per dose or 40 μg per dose of S1 subunit of the Spike protein of at least one variant SARS-CoV-2, and
i) the specific squalene or squalene oil-in-water adjuvant formulation at ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the first aspect or any of its embodiments, the protein subunit vaccine comprises between 5 to 50 μg per dose, preferably 10 μg per dose, 20 μg per dose or 40 μg per dose of the RBD antigen of the Spike protein of at least one variant SARS-CoV-2, and
i) MF59C.1 as adjuvant at a ratio (v/v) of 40-60% adjuvant and 60-40% antigen, preferably 50% adjuvant and 50% antigen, or
ii) 10-60 mg per dose, preferably 10 mg per dose, or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the first aspect or any of its embodiments, the protein subunit vaccine comprises between 5 to 50 μg per dose, preferably 10 μg per dose, 20 μg per dose or 40 μg per dose of the RBD antigen of the Spike protein of at least one variant SARS-CoV-2, and
i) the specific squalene or squalene oil-in-water adjuvant formulation at a ratio (v/v) of 40-60% adjuvant and 60-40% antigen, preferably 50% adjuvant and 50% antigen, or
ii) 10-60 mg per dose, preferably 10 mg per dose, or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the first aspect or any of its embodiments, the protein subunit vaccine comprises between 5 to 50 μg per dose, preferably 10 μg per dose, 20 μg per dose or 40 μg per dose of S1 subunit of the Spike protein of at least one variant SARS-CoV-2, and
i) an adjuvant comprising about 10 to 60 mg/ml of squalene per dose, 1 to 6 mg/ml of polysorbate 80 per dose, 1 to 6 mg/ml of sorbitan trioleate per dose, 0.5 to 6 mg/ml of sodium citrate per dose, and 0.01 to 0.5 mg/ml of citric acid per dose, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the first aspect or any of its embodiments, the protein subunit vaccine comprises between 5 to 50 μg per dose, preferably 10 μg per dose, 20 μg per dose or 40 μg per dose of the RBD antigen of the Spike protein of at least one variant SARS-CoV-2, and
i) an adjuvant comprising about 10 to 60 mg/ml of squalene per dose, 1 to 6 mg/ml of polysorbate 80 per dose, 1 to 6 mg/ml of sorbitan trioleate per dose, 0.5 to 6 mg/ml of sodium citrate per dose, and 0.01 to 0.5 mg/ml of citric acid per dose, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In an embodiment, the protein subunit vaccine according to the second aspect or any of its embodiments comprises or consists of at least an RBD antigen and at least one adjuvant, wherein the at least one adjuvant is MF59C.1. In a further embodiment, the protein subunit vaccine comprises or consists of at least a S1 subunit antigen and at least one adjuvant, wherein the at least one adjuvant is MF59C.1. In an embodiment, the protein subunit vaccine consists of at least an RBD antigen and MF59C.1 as adjuvant. In another embodiment, the protein subunit vaccine consists of at least a S1 subunit antigen and MF59C.1 as adjuvant.

In an embodiment, the protein subunit vaccine according to the second aspect or any of its embodiments comprises or consists of at least an RBD antigen and at least one adjuvant, wherein the at least one adjuvant is the specific squalene or squalene oil-in-water adjuvant formulation. In a further embodiment, the protein subunit vaccine comprises or consists of at least a S1 subunit antigen and at least one adjuvant, wherein the at least one adjuvant is the specific squalene or squalene oil-in-water adjuvant formulation. In an embodiment, the protein subunit vaccine consists of at least an RBD antigen and the specific squalene or squalene oil-in-water adjuvant formulation. In another embodiment, the protein subunit vaccine consists of at least a S1 subunit antigen and the specific squalene or squalene oil-in-water adjuvant formulation.

In an embodiment, the protein subunit vaccine according to the second aspect or any of its embodiments comprises or consists of at least an RBD antigen and at least one adjuvant, wherein the at least one adjuvant is AlPO₄ gel. In a further embodiment, the protein subunit vaccine comprises or consists of at least a S1 subunit antigen and at least one adjuvant, wherein the at least one adjuvant is AlPO₄ gel. In an embodiment, the protein subunit vaccine consists of at least an RBD antigen and AlPO₄ gel as adjuvant. In another embodiment, the protein subunit vaccine consists of at least a S1 subunit antigen and AlPO₄ gel as adjuvant.

In a preferred embodiment, the protein subunit vaccine according to the second aspect comprises or consists of between 5 to 50 μg per dose, preferably 10 μg per dose, 20 μg per dose or 40 μg per dose of S1 subunit of the Spike protein of at least one variant SARS-CoV-2, and
i) MF59C.1 as adjuvant at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant.

In a preferred embodiment, the protein subunit vaccine according to the second aspect comprises or consists of between 5 to 50 μg per dose, preferably 10 μg per dose, 20 μg per dose or 40 μg per dose of S1 subunit of the Spike protein of at least one variant SARS-CoV-2, and
i) the specific squalene or squalene oil-in-water adjuvant formulation at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant.

In a preferred embodiment, the protein subunit vaccine according to the second aspect or any of its embodiments comprises between 5 to 50 μg per dose, preferably 10 μg per dose, 20 μg per dose or 40 μg per dose of the receptor-binding domain (RBD) antigen of the Spike protein of at least one variant SARS-CoV-2, and
i) MF59C.1 as adjuvant at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant.

In a preferred embodiment, the protein subunit vaccine according to the second aspect or any of its embodiments comprises between 5 to 50 μg per dose, preferably 10 μg per dose, 20 μg per dose or 40 μg per dose of the receptor-binding domain (RBD) antigen of the Spike protein of at least one variant SARS-CoV-2, and
i) the specific squalene or squalene oil-in-water adjuvant formulation at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant.

In a preferred embodiment, the protein subunit vaccine according to the second aspect comprises or consists of between 5 to 50 μg per dose, preferably 10 μg per dose, 20 μg per dose or 40 μg per dose of S1 subunit of the Spike protein of at least one variant SARS-CoV-2, and
i) an adjuvant comprising about 10 to 60 mg/ml of squalene per dose, 1 to 6 mg/ml of polysorbate 80 per dose, 1 to 6 mg/ml of sorbitan trioleate per dose, 0.5 to 6 mg/ml of sodium citrate per dose, and 0.01 to 0.5 mg/ml of citric acid per dose, or
ii) 10-60 mg per dose, preferably 10 mg per dose or 50 mg per dose of AlPO₄ gel as adjuvant.

In a preferred embodiment, the protein subunit vaccine according to the second aspect or any of its embodiments comprises between 5 to 50 μg per dose, preferably 10 μg per dose, 20 μg per dose or 40 μg per dose of the receptor-binding domain (RBD) antigen of the Spike protein of at least one variant SARS-CoV-2, and
i) an adjuvant comprising about 10 to 60 mg/ml of squalene per dose, 1 to 6 mg/ml of polysorbate 80 per dose, 1 to 6 mg/ml of sorbitan trioleate per dose, 0.5 to 6 mg/ml of sodium citrate per dose, and 0.01 to 0.5 mg/ml of citric acid per dose, or
ii) 10-60 mg per dose, preferably 10 mg per dose or 50 mg per dose of AlPO₄ gel as adjuvant.

According to the second aspect or any of its embodiments, the at least one immunostimulant can be combined with the at least one adjuvant as described above. In a preferred embodiment of the second aspect or any of its embodiments, the protein subunit vaccine comprises or consists of MF59C.1 as adjuvant and MPLA as immunostimulant. In a preferred embodiment, the protein subunit vaccine comprises or consists of at least one RBD antigen, MF59C.1, and MPLA. In another preferred embodiment, the protein subunit vaccine comprises or consists of at least one S1 subunit antigen, MF59C.1, and MPLA. In a preferred embodiment, the protein subunit vaccine comprises or consists of at least one RBD antigen, MF59C.1, and QS-21. In another preferred embodiment, the protein subunit vaccine comprises or consists of at least one S1 subunit antigen, MF59C.1, and QS-21.

According to the second aspect or any of its embodiments, the at least one immunostimulant can be combined with the at least one adjuvant as described above. In a preferred embodiment of the second aspect or any of its embodiments, the protein subunit vaccine comprises or consists of the specific squalene or squalene oil-in-water adjuvant formulation and MPLA as immunostimulant. In a preferred embodiment, the protein subunit vaccine comprises or consists of at least one RBD antigen, the specific squalene or squalene oil-in-water adjuvant formulation, and MPLA. In another preferred embodiment, the protein subunit vaccine comprises or consists of at least one S1 subunit antigen, the specific squalene or squalene oil-in-water adjuvant formulation, and MPLA. In a preferred embodiment, the protein subunit vaccine comprises or consists of at least one RBD antigen, the specific squalene or squalene oil-in-water adjuvant formulation, and QS-21. In another preferred embodiment, the protein subunit vaccine comprises or consists of at least one S1 subunit antigen, the specific squalene or squalene oil-in-water adjuvant formulation, and QS-21.

In a preferred embodiment of the first aspect or any of its embodiments, the protein subunit vaccine comprises or consists of AlPO₄ gel as adjuvant and MPLA as immunostimulant. In a preferred embodiment, the protein subunit vaccine comprises or consists of at least one RBD antigen, AlPO₄₄ gel, and MPLA. In another preferred embodiment, the protein subunit vaccine comprises or consists of at least one S1 subunit antigen, AlPO₄ gel, and MPLA. In a preferred embodiment, the protein subunit vaccine comprises or consists of at least one RBD antigen, AlPO₄ gel, and QS-21. In another preferred embodiment, the protein subunit vaccine comprises or consists of at least one S1 subunit antigen, AlPO₄ gel, and QS-21.

In a preferred embodiment of the second aspect or any of its embodiments, the protein subunit vaccine comprises or consists of at least one RBD antigen, MF59C.1, MPLA and QS-21. In another preferred embodiment, the protein subunit vaccine comprises or consists of at least one S1 subunit antigen, MF59C.1, and MPLA and QS-21. In a preferred embodiment, the protein subunit vaccine comprises or consists of at least one RBD antigen, AlPO₄ gel, MPLA and QS-21. In another preferred embodiment, the protein subunit vaccine comprises or consists of at least one S1 subunit antigen, AlPO₄ gel, and MPLA and QS-21.

In a preferred embodiment of the second aspect or any of its embodiments, the protein subunit vaccine comprises or consists of at least one RBD antigen, the specific squalene or squalene oil-in-water adjuvant formulation, MPLA and QS-21. In another preferred embodiment, the protein subunit vaccine comprises or consists of at least one S1 subunit antigen, the specific squalene or squalene oil-in-water adjuvant formulation, and MPLA and QS-21. In a preferred embodiment, the protein subunit vaccine comprises or consists of at least one RBD antigen, AlPO₄ gel, MPLA and QS-21. In another preferred embodiment, the protein subunit vaccine comprises or consists of at least one S1 subunit antigen, AlPO₄ gel, and MPLA and QS-21.

In a preferred embodiment of the second aspect or any of its embodiments, the protein subunit vaccine comprises between 5 to 50 μg per dose, preferably 10 μg per dose, 20 μg per dose or 40 μg per dose of S1 subunit of the Spike protein of at least one variant SARS-CoV-2, and
i) MF59C.1 as adjuvant at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the second aspect or any of its embodiments, the protein subunit vaccine comprises between 5 to 50 μg per dose, preferably 10 μg per dose, 20 μg per dose or 40 μg per dose of S1 subunit of the Spike protein of at least one variant SARS-CoV-2, and
i) the specific squalene or squalene oil-in-water adjuvant formulation at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the second aspect or any of its embodiments, the protein subunit vaccine comprises between 5 to 50 μg per dose, preferably 10 μg per dose, 20 μg per dose or 40 μg per dose of the RBD antigen of the Spike protein of at least one variant SARS-CoV-2, and
i) MF59C.1 as adjuvant at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the second aspect or any of its embodiments, the protein subunit vaccine comprises between 5 to 50 μg per dose, preferably 10 μg per dose, 20 μg per dose or 40 μg per dose of the RBD antigen of the Spike protein of at least one variant SARS-CoV-2, and
i) the specific squalene or squalene oil-in-water adjuvant formulation at a ratio (v/v) of 40-60% adjuvant and 60-40% antigen, preferably 50% adjuvant and 50% antigen, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the second aspect or any of its embodiments, the protein subunit vaccine comprises between 5 to 50 μg per dose, preferably 10 μg per dose, 20 μg per dose or 40 μg per dose of S1 subunit of the Spike protein of at least one variant SARS-CoV-2, and
i) an adjuvant comprising about 10 to 60 mg/ml of squalene per dose, 1 to 6 mg/ml of polysorbate 80 per dose, 1 to 6 mg/ml of sorbitan trioleate per dose, 0.5 to 6 mg/ml of sodium citrate per dose, and 0.01 to 0.5 mg/ml of citric acid per dose, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

In a preferred embodiment of the second aspect or any of its embodiments, the protein subunit vaccine comprises between 5 to 50 μg per dose, preferably 10 μg per dose, 20 μg per dose or 40 μg per dose of the RBD antigen of the Spike protein of at least one variant SARS-CoV-2, and
i) an adjuvant comprising 10 to 60 mg/ml of squalene per dose, 1 to 6 mg/ml of polysorbate 80 per dose, 1 to 6 mg/ml of sorbitan trioleate per dose, 0.5 to 6 mg/ml of sodium citrate per dose, and 0.01 to 0.5 mg/ml of citric acid per dose, or
ii) 10-60 mg per dose, preferably 10 mg per dose or 50 mg per dose of AlPO₄ gel as adjuvant,
   and wherein the protein subunit vaccine further comprises at least one immunostimulant, wherein the at least one immunostimulant consists of:
a) 5-60 μg per dose, preferably 10 μg per dose or 50 μg per dose of MPLA, or
b) 5-60 μg per dose, preferably 10 μg per dose or 50 μg per dose of QS-21, or,
c) 5-30 μg per dose, preferably 5 μg per dose or 25 μg per dose of MPLA, and 5-30 μg per dose, preferably 5 μg per dose or 25 μg per dose of QS-21.

The vaccines described herein in the first or the second aspect or any of its embodiments may generally include one or more “pharmaceutically acceptable excipients or vehicles” such as water, saline, glycerol, ethanol, etc. Additionally, auxiliary substances, such as wetting or emulsifying agents, pH buffering substances, and the like, may be present in such vehicles. Typically, the protein subunit vaccines are prepared as injectables, either as liquid solutions or suspensions; solid forms suitable for solution in, or suspension in, liquid vehicles prior to injection may also be prepared. A carrier is optionally present which is a molecule that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. In another embodiment, the composition can be delivered in a vesicle, e.g., in a liposome. Methods of preparation of pharmaceutical formulations are well known by the person skilled in the art as it is described for example in the manual Remington The Science and Practice of Pharmacy, 20^{th} Ed., Lippincott Williams & Wilkins, Philadelphia, 2000 [ISBN: 0-683-306472].

### Administration Regimen

The routes and schedule of administration can be chosen and optimized by those skilled in the art in a known manner.

Administration routes can be systemic or local. Many methods of administration may be used including but not limited to oral, via parenteral (e.g. intradermal, intramuscular, intravenous and subcutaneous), via transdermal, via mucosal (e.g., intranasal and oral or pulmonary routes or by vaginal suppositories), via pulmonary delivery, via suppository, via scarification (scratching through the top layers of skin, e.g., using a bifurcated needle). In a specific embodiment, the protein subunit vaccine of the present invention is administered parenterally via intramuscular, intravenous, intradermal or subcutaneous route, or alternatively is administered by transdermal route. Preferably, the said protein subunit vaccine is administered by intramuscular or subcutaneous route. More preferably, the said protein subunit vaccine is administered intramuscularly in a volume ranging between about 0.10 ml and 10 ml, or between 0.10 ml and 1 ml. Preferably, the said protein subunit vaccine is administered in a volume ranging between 0.25 ml and 1.0 ml. More preferably, the said protein subunit vaccine is administered in a volume of about 0.1 ml. Even more preferably, the said protein subunit vaccine is administered in a volume of about 0.5 ml.

In certain embodiments, the protein subunit vaccine provided in the first or the second aspect or any of its embodiments is administered to a subject following a vaccine protocol or schedule that comprises a single dose, or alternatively multiple (i.e., 2, 3, 4, etc.) doses. Preferably, the protein subunit vaccine is administered to the subject in need thereof in two doses. In certain embodiments, the said protein subunit vaccine is administered to the subject in need thereof in a schedule comprising a first dose (priming) and a second dose (boosting).

Priming, as used herein, means any method whereby a first administration of the protein subunit vaccine described herein permits the generation of an immune response to a target antigen or antigens. Once the subject is primed, a second administration with a second vaccine induces a second immune response that is greater or longer in duration than that achieved with the first immunization. Priming encompasses regimens which include a first single dose or multiple dosages. In an embodiment, a first infection with the SARS-CoV-2 can be considered as a priming immunization, and a single dose of the protein subunit vaccine is administered for the first time as a booster.

The time interval between priming and boosting administrations can be hours, days, weeks, months or years. In other embodiments, the protein subunit vaccine described herein may be administered as a booster to increase the immune response achieved after priming of the subject. Boosting compositions are generally administered once or multiple times weeks or months after administration of the priming composition, for example, about 1 or 2 weeks or 3 weeks, or 4 weeks, or 6 weeks, or 8 weeks, or 16 weeks, or 20 weeks, or 24 weeks, or 28 weeks, or 32 weeks or one to two years. Preferably, the boosting inoculation is administered 1-12 weeks or 2-12 weeks after priming, more preferably 1, 2, 3, or 4 weeks after priming. In a preferred embodiment, the second dose or the boosting dose is administered one, preferably two, three or four weeks after the first dose or priming. In additional embodiments, the second dose is conducted at least 2 weeks or at least 4 weeks after priming. In still another preferred embodiment, the second dose is conducted about 4-12 weeks or 4-8 weeks after priming.

Additionally, a third or subsequent boosting doses may be administered after the second dose and from three months to two years, or even longer, preferably 4 to 6 months, or 6 months to one year after the initial administration. The third dose may be optionally administered when no or low levels of specific immunoglobulins are detected in the serum and/or urine or mucosal secretions of the subject after the second dose.

In an embodiment, the protein subunit vaccine provided herein can be administered as prime and as the subsequent booster or boosters. In other embodiments, said protein subunit vaccine can be used for priming and/or for boosting in combination with other vaccines, such as mRNA vaccines, plasmid vaccines, vector vaccines, other protein subunit vaccines, or combinations thereof.

In an embodiment, the protein subunit vaccine provided in the first or the second aspect or any of its embodiments can be administered in a single dose as a booster in subjects that have been previously vaccinated with the protein subunit vaccine provided herein or with other vaccines. In this case, the protein subunit vaccine provided herein is administered one, two, three, for, five, six, seven, eight, nine, ten or more than ten weeks, months or years after the previous vaccines have been administered to the subject.

In some embodiments, the protein subunit vaccine provided herein is administered to the subject at any of the doses, routes, or schedules as defined herein prior SARS-CoV-2 virus exposure as, e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours or 1 day, 2 days, 3 days, 4 days, 5 days, 6 days, or 7 days prior SARS-CoV-2 exposure. In certain embodiments, the protein subunit vaccine provided herein is administered to the subject at any of the doses, routes or schedules as defined herein after SARS-CoV-2 virus exposure as, e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours or 1 day, 2 days, 3 days, 4 days, 5 days, 6 days, or 7 days, or 1 week or 2 weeks or 3 weeks, or 4 weeks, or 6 weeks, or 8 weeks, or 16 weeks, or weeks, or 24 weeks, or 28 weeks, or 32 weeks after SARS-CoV-2 exposure.

### Kits

Also provided herein are kits comprising the protein subunit vaccine of the first or the second aspect or any of its embodiments. Thus, a third aspect of the invention refers to kits comprising one, preferably two, or more doses of the protein subunit vaccine as defined in the first or the second aspect of the invention or any of its embodiments. The kits may therefore include the at least one antigen, the at least one adjuvant and, optionally, the at least one immunostimulant as defined in the first or the second aspect or any of its embodiments. Each component of said protein subunit vaccine can be provided independently, that is, separated in individual containers, or mixed, that is, all together in one or more containers.

Thus, in an embodiment, the kit can comprise one or multiple containers or vials of the protein subunit vaccine of the present invention, or one or multiple containers or vials of the protein subunit vaccine together with instructions for the administration to a subject at risk of SARS-CoV-2 infection. In certain embodiments, the instructions indicate that the protein subunit vaccine of the present invention is administered to the subject in a single dose, or in multiple (i.e., 2, 3, 4, etc.) doses as defined above in the administration schedule section. In certain embodiments, the instructions indicate that the protein subunit vaccine of the present invention is administered in a first (priming) and subsequent (boosting) administrations to naive or non-naive subjects. Preferably, the kit comprises at least two vials for prime/boost immunization comprising the protein subunit vaccine of the present invention for a first inoculation or first dose (“priming inoculation”) in a first vial/container and for an at least second and/or third and/or further inoculation or dose (“boosting inoculation”) in a second and/or further vial/container.

Preferably, the kit comprises an immunologically effective amount of the protein subunit vaccine according to the first or the second aspect of the invention or any of its embodiments in a first vial or container for a first administration or first dose (priming) and in a second vial or container for a second administration or second dose (boosting).

In another embodiment of the second aspect of the invention, any of the kits referred to herein, may comprise a third, fourth or further vial or container comprising the protein subunit vaccine indicated throughout the present invention for a third, fourth or further administration.

In a further preferred embodiment, the protein subunit vaccine and kits provided in any of the previous aspects are for use in generating an immune response against at least one variant of the SARS-CoV-2 virus.

In another preferred embodiment, the protein subunit vaccine and kits provided in any of the previous aspects are for use in generating a protective immune response against at least one variant of SARS-CoV-2 virus.

### Methods and Uses of the Protein Subunit Vaccine

In a fourth aspect, the present invention also provides methods of use of the protein subunit vaccine and the kits as described in the first, second and third aspects of the invention or any of their embodiments, for immunizing a subject against at least one variant of the SARS-CoV-2 virus. The fourth aspect also relates to the use of the protein subunit vaccine and the kits as described in the first, second and third aspects or any of their embodiments for generating an immunogenic and/or protective immune response against at least one variant of the SARS-CoV-2 virus in a subject in need thereof. Preferably, the fourth aspect relates to the use of the protein subunit vaccine and the kits as described in the first, second and third aspects or any of their embodiments for generating an immunogenic and/or protective immune response against at least one different variants of the SARS-CoV-2 virus in a subject in need thereof, wherein the variants are selected from the variants of concern (VOC), as described by the Centers for Disease Control and Prevention (CDC). Preferably, the fourth aspect relates to the use of the protein subunit vaccine and the kits as described in the first, second and third aspects or any of their embodiments for generating an immunogenic and/or protective immune response against at least one different variants of the SARS-CoV-2 virus in a subject in need thereof, wherein the variants are selected from the group comprising or consisting of Wuhan-Hu-1 seafood market pneumonia virus isolate (GenBank accession number: MN908947), Linage B.1.1.28 (Brazilian variant), Linage B.1.351 (South Africa variant), Linage B.1.427 or Linage B.1.429 (California variant), Linage B.1.617 (Indian variant), Linage B.1.1.7 (United Kingdom variant), Linage B.1.617.2 or G/478K.V1 (Delta Variant) or Linage B.1.1.529 or GR/484A (Omicron variant) or any combination thereof. Preferably, the fourth aspect relates to the use of the protein subunit vaccine and the kits as described in the first, second and third aspects or any of their embodiments for generating an immunogenic and/or protective immune response against at least two different variants of the SARS-CoV-2 virus in a subject in need thereof. Preferably, the fourth aspect relates to the use of the protein subunit vaccine and the kits as described in the first, second and third aspects or any of their embodiments for generating an immunogenic and/or protective immune response against at least two different variants of the SARS-CoV-2 virus in a subject in need thereof, wherein the variants are selected from the variants of concern (VOC), as described by the Centers for Disease Control and Prevention (CDC). Preferably, the fourth aspect relates to the use of the protein subunit vaccine and the kits as described in the first, second and third aspects or any of their embodiments for generating an immunogenic and/or protective immune response against at least two different variants of the SARS-CoV-2 virus in a subject in need thereof, wherein the variants are selected from the group comprising or consisting of Wuhan-Hu-1 seafood market pneumonia virus isolate (GenBank accession number: MN908947), Linage B.1.1.28 (Brazilian variant), Linage B.1.351 (South Africa variant), Linage B.1.427 or Linage B.1.429 (California variant), Linage B.1.617 (Indian variant), Linage B.1.1.7 (United Kingdom variant), Linage B.1.617.2 or G/478K.V1 (Delta Variant) or Linage B.1.1.529 or GR/484A (Omicron variant) or any combination thereof.

Also included are uses of the said protein subunit vaccine and kits described above for the preparation of a medicament or protein subunit vaccines for the immunization of a subject, in particular for the preparation of a medicament or vaccine for treating and/or preventing a SARS-CoV-2-caused disease in a subject, wherein the SARS-CoV-2 disease is caused by at least one variant of the SARS-CoV-2 virus. Provided are also herein the said protein subunit vaccine and kit according to any embodiment herein for use in priming or boosting an immune response against a SARS-CoV-2 infection, wherein the protein subunit vaccine is administered once, twice, three or four times. Preferably, the protein subunit vaccine is administered twice. Provided are also herein the said protein subunit vaccine and kit according to any embodiment herein for use in boosting an immune response against a SARS-CoV-2 infection in subjects that have been previously vaccinated against SARS-CoV-2, wherein the protein subunit vaccine is administered in a single dose.

Accordingly, the fourth aspect of the present invention also provides a method of generating an immunogenic and/or protective immune response against at least one variant of the SARS-CoV-2 virus in a subject in need thereof, preferably in a human subject, the method comprising administering to the subject the protein subunit vaccine as described in the first or the second aspect of the invention or any of its embodiments. The terms “immunogenic and protective immune response”, “protective immunity” or “protective immune response” have been defined above.

In certain embodiments, the subject is a mammal or an avian species. The subject may be a human, a companion animal such as dogs and cats, a domestic animal such as chicken and geese, horses, cattle and sheep, ferrets, porcine species such as pigs, piglets, sow or gilts, and zoo mammals such as non-human primates, felids, canids and bovids.

In an embodiment the method comprises administering at least one dose of the protein subunit vaccine of the present invention to the subject, preferably the subject is a human.

In certain embodiments of the fourth aspect of the invention, the subject is a human. In certain embodiments, the subject is a neonate (up to 2 months of age), an infant (birth to 2 years of age), a child (2 years to 14 years of age), a teenager (15 years to 18 years of age), an adult (above 18 years of age), or a senior adult (about 65 years of age or older). In certain embodiments, the adult is immune-compromised.

In a further embodiment, the protein subunit vaccine or the kit as defined in the first, second or third aspects of the present invention or any of its embodiments, is for use in generating an immunogenic and/or protective immune response against at least one variant of SARS-CoV-2 in a subject.

In a further embodiment, the protein subunit vaccine or the kit as defined in the first, second or third aspects of the present invention or any of its embodiments, is for use in generating an immunogenic and/or protective immune response against at least two different variants of SARS-CoV-2 in a subject.

SEQUENCE LISTING

SEQ ID NO 1: RBD monomer: amino acid residues 319 to 541 of the SARS-CoV-2 Spike
protein (Wuhan variant):
RVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQ
PTNGVGYQPYRVVVLSFELLHAPATVCGPKKSTNLVKNKCVNF
SEQ ID NO 2: S1 subunit monomer: amino acid residues 13 to 685 of the SARS-CoV-2 Spike
protein (Wuhan variant):
SQCVNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNFKNLREF
VFKNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTVEKGI
YQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNCYFPL
QSYGFQPTNGVGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGICA
SYQTQTNSPRRAR
SEQ ID NO 3: Amino acid residues 319 to 537 of the SARS-CoV-2 Spike protein RBD
monomer of the B1.1.7 variant:
RVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFPLQSYGFQPTYGV
GYQPYRVVVLSFELLHAPATVCGPKKSTNLVKNK
SEQ ID NO 4: Amino acid residues 319 to 537 of the SARS-CoV-2 Spike protein RBD
monomer of the B.1.351 variant:
RVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGNIADYNYKLPDDFTGCVIAWNSNNLD
SKVGGNYNYLYRLFRKSNLKPFERDISTEIYQAGSTPCNGVKGFNCYFPLQSYGFQPTYGV
GYQPYRVVVLSFELLHAPATVCGPKKSTNLVKNK
SEQ ID NO 5: Amino acid sequence of the fusion dimeric RBD variant SARS-CoV-2 antigen
comprising a first monomer derived from the B.1.351 variant, positions 319 to 537 of the RBD
of the SARS-CoV-2 Spike protein and a second monomer derived from the B.1.1.7 variant,
positions 319 to 537 of the RBD of the SARS-CoV-2 Spike protein:
RVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGNIADYNYKLPDDFTGCVIAWNSNNLD
SKVGGNYNYLYRLFRKSNLKPFERDISTEIYQAGSTPCNGVKGFNCYFPLQSYGFQPTYGV
GYQPYRVVVLSFELLHAPATVCGPKKSTNLVKNKRVQPTESIVRFPNITNLCPFGEVFNATR
FASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSNLKPFERDIST
EIYQAGSTPCNGVEGFNCYFPLQSYGFQPTYGVGYQPYRVVVLSFELLHAPATVCGPKKST
NLVKNK
SEQ ID NO 6: Signal peptide:
MGWSCIILFLVATATGVHS
SEQ ID NO 7: DNA sequence encoding the Kozak sequence, the signal peptide, the fusion
dimeric RBD variant SARS-CoV-2 antigen which is a tandem of the nucleotide sequence
encoding the amino acid positions 319 to 537 of the RBD monomer of the B.1.351 variant and
the amino acid positions 319 to 537 of the RBD monomer of the B.1.1.7 variant, the histidine
tag and the stop codon:
GCCACCATGGGCTGGTCCTGCATCATCCTGTTTCTGGTGGCTACCGCTACCGGCGTGC
ACAGTAGAGTGCAGCCTACCGAGTCTATCGTGCGGTTCCCCAACATCACCAACCTGTGT
CCTTTCGGCGAGGTGTTCAACGCCACCAGATTCGCCTCTGTGTACGCCTGGAACCGGA
AGCGGATCTCTAACTGCGTGGCCGACTACTCCGTGCTGTACAACTCCGCCTCCTTCAGC
ACCTTCAAGTGCTACGGCGTGTCCCCTACCAAGCTGAACGACCTGTGCTTCACCAACGT
GTACGCCGACTCCTTCGTGATCAGAGGCGACGAAGTGCGGCAGATCGCTCCTGGACAG
ACCGGCAATATCGCCGACTACAACTACAAGCTGCCCGACGACTTCACCGGCTGTGTGA
TCGCTTGGAACTCCAACAACCTGGACTCCAAAGTCGGCGGCAACTACAATTACCTGTAC
CGGCTGTTCCGGAAGTCCAACCTGAAGCCTTTCGAGCGGGACATCTCCACCGAGATCT
ACCAGGCTGGCAGCACCCCTTGTAATGGCGTGAAGGGCTTCAACTGCTACTTCCCACT
GCAGTCCTACGGCTTCCAGCCTACCTATGGCGTGGGCTACCAGCCTTACAGAGTGGTG
GTGCTGTCCTTCGAGCTGCTGCATGCTCCTGCTACCGTGTGCGGCCCTAAGAAATCTAC
CAACCTGGTCAAGAACAAGCGGGTGCAGCCCACTGAGAGCATTGTGCGCTTCCCTAAT
ATCACAAATCTGTGCCCCTTCGGGGAAGTCTTTAATGCTACCCGCTTCGCTTCCGTGTA
TGCTTGGAATAGAAAGCGGATCAGCAATTGCGTCGCCGATTACAGCGTCCTGTACAATA
GCGCCAGCTTCTCCACCTTTAAGTGTTATGGCGTCAGCCCCACAAAGCTCAACGATCTC
TGTTTTACCAATGTCTACGCCGATAGCTTTGTGATTCGCGGAGATGAAGTCCGCCAGAT
CGCACCAGGCCAGACTGGAAAGATCGCTGATTACAATTATAAGCTCCCTGATGATTTCA
CAGGATGCGTTATCGCCTGGAATAGCAACAACCTCGACAGCAAAGTTGGAGGGAATTAC
AACTACCTCTACCGCCTCTTCAGAAAGAGCAACCTCAAGCCATTTGAGAGAGACATCAG
TACAGAAATCTATCAGGCCGGCTCTACCCCTTGCAACGGCGTCGAGGGGTTTAACTGTT
ACTTTCCCCTGCAATCTTATGGGTTTCAGCCCACATACGGCGTGGGGTATCAACCCTAT
CGCGTGGTGGTTCTGAGTTTCGAACTCCTGCACGCCCCAGCCACAGTGTGTGGCCCAA
AAAAGAGCACCAATCTCGTTAAGAACAAGCACCATCACCATCACCATTAG
SEQ ID NO 8: DNA sequence encoding the fusion dimeric RBD variant SARS-CoV-2 antigen,
which is tandem of the nucleotide sequence that encode the amino acid positions 319 to 537
of the RBD monomer of the B.1.351 variant and the amino acid positions 391 to 537 of the
RBD monomer of the B.1.1.7 variant:
AGAGTGCAGCCTACCGAGTCTATCGTGCGGTTCCCCAACATCACCAACCTGTGTCCTTT
CGGCGAGGTGTTCAACGCCACCAGATTCGCCTCTGTGTACGCCTGGAACCGGAAGCGG
ATCTCTAACTGCGTGGCCGACTACTCCGTGCTGTACAACTCCGCCTCCTTCAGCACCTT
CAAGTGCTACGGCGTGTCCCCTACCAAGCTGAACGACCTGTGCTTCACCAACGTGTAC
GCCGACTCCTTCGTGATCAGAGGCGACGAAGTGCGGCAGATCGCTCCTGGACAGACC
GGCAATATCGCCGACTACAACTACAAGCTGCCCGACGACTTCACCGGCTGTGTGATCG
CTTGGAACTCCAACAACCTGGACTCCAAAGTCGGCGGCAACTACAATTACCTGTACCGG
CTGTTCCGGAAGTCCAACCTGAAGCCTTTCGAGCGGGACATCTCCACCGAGATCTACCA
GGCTGGCAGCACCCCTTGTAATGGCGTGAAGGGCTTCAACTGCTACTTCCCACTGCAG
TCCTACGGCTTCCAGCCTACCTATGGCGTGGGCTACCAGCCTTACAGAGTGGTGGTGC
TGTCCTTCGAGCTGCTGCATGCTCCTGCTACCGTGTGCGGCCCTAAGAAATCTACCAAC
CTGGTCAAGAACAAGCGGGTGCAGCCCACTGAGAGCATTGTGCGCTTCCCTAATATCA
CAAATCTGTGCCCCTTCGGGGAAGTCTTTAATGCTACCCGCTTCGCTTCCGTGTATGCT
TGGAATAGAAAGCGGATCAGCAATTGCGTCGCCGATTACAGCGTCCTGTACAATAGCGC
CAGCTTCTCCACCTTTAAGTGTTATGGCGTCAGCCCCACAAAGCTCAACGATCTCTGTTT
TACCAATGTCTACGCCGATAGCTTTGTGATTCGCGGAGATGAAGTCCGCCAGATCGCAC
CAGGCCAGACTGGAAAGATCGCTGATTACAATTATAAGCTCCCTGATGATTTCACAGGA
TGCGTTATCGCCTGGAATAGCAACAACCTCGACAGCAAAGTTGGAGGGAATTACAACTA
CCTCTACCGCCTCTTCAGAAAGAGCAACCTCAAGCCATTTGAGAGAGACATCAGTACAG
AAATCTATCAGGCCGGCTCTACCCCTTGCAACGGCGTCGAGGGGTTTAACTGTTACTTT
CCCCTGCAATCTTATGGGTTTCAGCCCACATACGGCGTGGGGTATCAACCCTATCGCGT
GGTGGTTCTGAGTTTCGAACTCCTGCACGCCCCAGCCACAGTGTGTGGCCCAAAAAAG
AGCACCAATCTCGTTAAGAACAAG
SEQ ID NO 9: Spike protein sequence of the Wuhan-Hu-1 SARS-CoV-2 (UniProt No.
P0DTC2):
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT
SEQ ID NO 10: Signal peptide.
MGWSLILLFLVAVATRVLS

Other embodiments of the invention will be apparent to those skilled in the art from consideration of the specification and practice of the invention disclosed herein. It is intended that the specification and examples be considered as exemplary only, with a true scope and spirit of the invention being indicated by the appended claims.

### EXAMPLES

### Example 1: In Vivo Immunization Study in Mice Testing 2 Different Antigen Candidates Combined with 4 Different Adjuvants

This study evaluated different protein subunit vaccine candidates against SARS-CoV-2. The study assessed different recombinant subunit antigens of SARS-CoV-2 in different vaccine formulations and the immunogenic capabilities of the different protein subunit vaccine formulations in mice.

A total of 86 BALB/c mice of 6-7 weeks of age were selected for the study. Mice were allotted into 9 different Groups which received a different vaccine formulation. Ten mice were allotted in each group with exception of the control Group (Group A) in which 6 mice were included. Animals received two doses of 0.1 ml of the following vaccine formulations administered subcutaneously according to the treatment Group. The vaccines were formulated as a “ready-to-use” vaccine. The first dose was administered to the animals on Day 0 and the second dose 3 weeks apart (Day 21).
Group A: this group was the control group. The animals in this group received a mock-vaccine comprising PBS.
Group B: animals in this group received a vaccine comprising 20 μg of a recombinant RBD antigen of SARS-CoV-2 per dose. The vaccine was formulated with an oil-in-water adjuvant in a dose of 0.1 ml at a ratio v/v 75% adjuvant and 25% antigen. The oil-in-water adjuvant was formulated as follows: 19.5 mg/ml of squalene, 2.35 mg/ml of polysorbate 80, 2.35 mg/ml of sorbitan trioleate, 1.32 mg/ml of sodium citrate and 0.08 mg/ml of citric acid. Thus, the 0.1 ml dose of the vaccine administered comprised 1.46 mg of squalene, 0.18 mg of polysorbate 80, 0.18 mg of sorbitan trioleate, 0.099 mg of sodium citrate and 0.006 mg of citric acid.
Group C: animals in this group received a vaccine comprising 20 μg of a recombinant RBD antigen of SARS-CoV-2 per dose. The vaccine was formulated with the same adjuvant as Group B, in a dose of 0.1 ml of the vaccine composition at a ratio v/v 75% adjuvant and 25% antigen, together with 10 μg/dose of MPLA immunostimulant (L6895, SIGMA) which was added into the antigenic phase.
Group D: animals in this group received a vaccine comprising 20 μg of a recombinant RBD antigen of SARS-CoV-2 per dose. The vaccine was formulated with 10 mg per dose of the adjuvant AlPO4 gel (Adju-Phos CRODA), in a dose of 0.1 ml of the vaccine composition at a ratio v/v 75% adjuvant and 25% antigen, together with 10 μg/dose of MPLA immunostimulant (L6895, SIGMA) which was added into the antigenic phase.
Group E: animals in this group received a vaccine comprising 20 μg of a recombinant RBD antigen of SARS-CoV-2 per dose. The vaccine was formulated with 10 mg per dose of the adjuvant AlPO4 gel (Adju-Phos CRODA), in a dose of 0.1 ml of the vaccine composition at a ratio v/v 75% adjuvant and 25% antigen, together with 5 μg/dose of the immunostimulant MPLA (L6895, SIGMA) and 5 μg/dose of immunostimulant QS-21 (QS-21, DESERT KING) which were added into the antigenic phase.
Group F: animals in this group received a vaccine comprising 20 μg of a recombinant S1 antigen of SARS-CoV-2 per dose. The vaccine was formulated with the same adjuvant as Group B, in a dose of 0.1 ml of the vaccine composition at a ratio v/v 75% adjuvant and 25% antigen.
Group G: animals in this group received a vaccine comprising 20 μg of a recombinant S1 antigen of SARS-CoV-2 per dose. The vaccine was formulated with the same adjuvant as Group B, in a dose of 0.1 ml of the vaccine composition at a ratio v/v 75% adjuvant and 25% antigen, together with 10 μg/dose of MPLA immunostimulant (L6895, SIGMA) which was added into the antigenic phase.
Group H: animals in this group received a vaccine comprising 20 μg of a recombinant S1 antigen of SARS-CoV-2 per dose. The vaccine was formulated with 10 mg per dose of the adjuvant AlPO4 gel (Adju-Phos, CRODA) in a dose of 0.1 ml of the vaccine composition at a ratio v/v 75% adjuvant and 25% antigen, together with 10 μg/dose of MPLA immunostimulant (L6895, SIGMA) which was added into the antigenic phase.
Group I: animals in this group received a vaccine comprising 20 μg of a recombinant S1 antigen of SARS-CoV-2 per dose. The vaccine was formulated with 10 mg per dose of the adjuvant AlPO4 gel (Adju-Phos, CRODA) in a dose of 0.1 ml of the vaccine composition at a ratio v/v 75% adjuvant and 25% antigen, together with 5 μg/dose of the immunostimulant MPLA (L6895, SIGMA) and 5 μg/dose of immunostimulant QS-21 (QS-21, DESERT KING) which were added into the antigenic phase.

The SARS-CoV-2 RBD sequence for producing the RBD subunit antigen used in this study consisted of positions 319 to 541 of the SARS-CoV-2 Spike glycoprotein (UniProt No. P0DTC2).

The SARS-CoV-2 S1 sequence for producing the S1 subunit antigen used in this study consisted of positions 16 to 682 of the SARS-CoV-2 Spike glycoprotein (UniProt No. P0DTC2).

For the preparation of the vaccine, the RBD and S1 encoding genes were codon-optimized for CHO-cell expression and further cloned into the expression plasmid pD2610-v10 (transient expression vector, from ATUM) with an N-terminal signal peptide of sequence MGWSLILLFLVAVATRVLS (SEQ ID NO: 10) for transient expression, and a C-terminal six histidine tag.

The oil-in-water adjuvant was produced by dispersing the sorbitan trioleate in squalene for obtaining the oil phase. Then, the aqueous phase was obtained by mixing the polysorbate 80 in an aqueous buffer of sodium citrate in citric acid. Both the oil and the aqueous phase were filtered before performing a high-speed mixing to form an oil-in-water emulsion of uniform small droplet size below 1 μm.

In this example, the oil-in-water adjuvant was produced to obtain an oil-in-water adjuvant formulation of 19.5 mg/ml of squalene, 2.35 mg/ml of polysorbate 80, 2.35 mg/ml of sorbitan trioleate, 1.32 mg/ml of sodium citrate and 0.08 mg/ml of citric acid The SARS-CoV-2 RBD and S1 antigens were produced in the ExpiCHO-S cell line (ThermoFisher). The ExpiCHO-S cell line were cultured in ExpiCHO Expression medium (ThermoFisher) at 37° C., 80% humidity and 8% CO₂, with agitation at 125 rpm for expansion. Then, the cells at a density of 6×10⁶ cells/ml were transiently transfected with 1 μg/ml of the expression plasmid pre-mixed with ExpiFectamine CHO Reagent (ThermoFisher) in OptiPRO SFM complexation medium (ThermoFisher). At 5 days of culture, the cells were removed by centrifugation at 300 g at room temperature for 5 minutes, and the supernatant was preserved.

The supernatant was then purified by immobilized metal-affinity chromatography with the 5 ml column HiScreen Ni FF (Cytiva). The target protein was eluted with buffer comprising 20 mM sodium phosphate, 500 mM NaCl, 500 mM imidazole at pH 7.2. The purified target protein was dialyzed with PBS (10 kDa RC membrane), concentrated at 1 mg/ml and filtered using PES filter of 0.22 μm pore size (Millex-GP) and stored at −80° C. for further use.

To assess the immunological response against SARS-CoV-2 after vaccinating the animals, two different parameters were analyzed: (i) Neutralizing antibodies in sera, and (ii) Cellular response (cytokines and active lymphocytes). Results are provided in Example 2.

### Example 2: Assessment of Neutralizing Antibodies and Immunological Cellular Response

Neutralizing antibodies in sera were assessed. For this, sera samples were extracted between and 21 days after the second dose of the vaccine (Day 40-41) from all vaccinated animals.

Neutralizing antibodies in sera were determined by a pseudovirus neutralization assay (PBNA) using a SARS-CoV-2 pseudovirus, as described in Nie J. et al. Quantification of SARS-CoV-2 neutralizing antibody by a pseudotyped virus-based assay. Nat Protoc. 2020 November; 15(11):3699-3715. A pseudovirus expressing SARS-CoV-2 S protein, and luciferase was generated for this assay.

For neutralization assay, 200 TCID₅₀ of pseudovirus supernatant were preincubated with serial dilutions of the heat-inactivated serum samples for 1 h at 37° C. and then added onto human ACE-2 overexpressing HEK293T cells. After 48 h, cells were lysed with Britelite Plus Luciferase reagent (Perkin Elmer, Waltham, MA, USA). Luminescence was measured for 0.2 s with an EnSight Multimode Plate Reader (Perkin Elmer). All assays were done in duplicate wells. Neutralization capacity of the plasma samples was calculated by comparing the experimental RLU (Relative Light Unit) calculated from infected cells treated with each plasma to the max RLUs (maximal infectivity calculated from untreated infected cells) and min RLUs (minimal infectivity calculated from uninfected cells), and expressed as percent neutralization:

$$\% Neutralization = \frac{( RLU max - RLUexperimental )}{( RLU max - RLU min )} * 100$$

Normalized dose response neutralization curves were fitted to a four-parameter curve with a variable slope using Graph Pad Prism (v8.3.0). All IC₅₀ values are expressed as reciprocal dilution (concentration required to inhibit 50% of infection).

Samples were tested at the following dilutions: 1/60, 1/180, 1/540, 1/1620, 1/4860 and 1/14580. Neutralization titers between 60 and 14580 can be quantified. Lower and higher titers below or above the limit of quantification are indicated as <60 and >14580, respectively.

For the assessment of the cellular immune response, animals were euthanized 21 days after receiving the second vaccine dose. Then, the spleens were extracted in order to obtain splenocytes. Splenocytes were stimulated in vitro with the antigen (RBD or S1) corresponding to that present in the vaccine formulation, depending on the treatment group from which splenocytes are derived. Splenocytes were cultured between 66 to 72 hours after the stimulation. Then, the concentration of the cultures of the obtained cytokines INF-γ, IL-4, IL-10, and IL-6 was determined in the supernatant. The cytokine concentration (pg/ml) in the supernatant of the cell cultures was determined by a standard ELISA technique.

Results of neutralizing antibodies in sera showed a higher titer of neutralizing antibodies in all vaccinated Groups compared to the control Group (Group A). The results show that vaccines comprising the RBD antigen induced a higher humoral response, with higher neutralizing antibodies, than vaccines comprising the S1 antigen (FIG. 1). The humoral response of most of the animals in the Groups vaccinated with a vaccine comprising the RBD antigen (Groups B to E) was above the limit of quantification of the PBNA assay in the range of dilutions tested (>14580). This result surprisingly showed a strong capacity to generate neutralizing antibodies for all the vaccines tested, in particular for the vaccines comprising the RBD antigen.

Regarding the cellular immune response, it was clearly observed that the selection of the adjuvant plays a key role in obtaining a strong immune response (FIG. 2 A-H). The Groups that received a vaccine comprising an oil-in-water adjuvant formulated as 19.5 mg/ml of squalene, 2.35 mg/ml of polysorbate 80, 2.35 mg/ml of sorbitan trioleate, 1.32 mg/ml of sodium citrate and 0.08 mg/ml of citric acid and MPLA as immunostimulant (Groups C and G), regardless whether they were treated with the RBD antigen or the S1 antigen, surprisingly showed a higher production in cytokines in the culture of splenocytes stimulated with the corresponding antigen in comparison with the rest of the groups. The results also showed that further including an immunostimulant, particularly MPLA, to the oil-in-water adjuvant (Group C and G) significantly enhances the production of cytokines (IFN-γ, IL-4, and IL-6) in comparison to the vaccine comprising only the oil-in-water adjuvant (Groups B and F). It was also demonstrated that including the immunostimulant QS-21 to a formulation comprising AlPO4 gel and MPLA (Groups E and I) also increases the cytokine production (IFN-γ, IL-4, and IL-6) compared to the formulation comprising only AlPO₄ and MPLA (Groups D and H), regardless whether the animals were treated with the RBD antigen or the S1 antigen.

It has been described that “Antibody-dependent enhancement” (ADE) in a SARS-CoV-2 infection is associated with high production of IL-6 by macrophages and a reduction in production of IL-10 (Iwasaki and Yang, 2020). Thus, the study also shows that the oil-in-water adjuvant and MPLA formulation would be suitable for reducing the risk of ADE in vaccinated people who could be exposed to the virus, because it has shown an increased IFN-γ/IL-6 cytokine ratio and also a high IL-10 production after splenocyte stimulation.

Overall, the results indicate that a subunit vaccine against SARS-CoV-2 that comprise a subunit RBD antigen or S1 antigen of SARS-CoV-2 together with an adjuvant and also further comprising an immunostimulant provides a higher immunological response to the subjects.

As mentioned before, both the oil-in-water adjuvant (formulated as 19.5 mg/ml of squalene, 2.35 mg/ml of polysorbate 80, 2.35 mg/ml of sorbitan trioleate, 1.32 mg/ml of sodium citrate and 0.08 mg/ml of citric acid) and MPLA are used in human vaccines. From the results it is observed that the oil-in-water adjuvant is sufficient to induce an immune response against SARS-CoV-2 to the vaccinated subjects. This adjuvant is also suitable to be used in a vaccine composition due to its known safety profile. Furthermore, when an immunostimulant is further included to the oil-in-water adjuvanted vaccine formulation the immune response is considerably increased. Therefore, the use of an immunostimulant to the vaccine composition further increases the immune response to the subject which receives said vaccine composition.

### Example 3: Antigen Production and Analysis of Convalescent Human Serum Collections

The SARS-CoV-2 RBD sequence for producing the RBD subunit antigen consisted of positions 319 to 541 of the SARS-CoV-2 Spike glycoprotein (UniProt No. P0DTC2).

For the preparation of the vaccine, the RBD encoding gene was codon-optimized for CHO-cell and HEK293 cell expression and further cloned into the expression plasmid pD2610-v10 (transient expression vector from ATUM) with an N-terminal signal peptide of sequence MGWSLILLFLVAVATRVLS (SEQ ID NO: 10) for transitory expression, and a C-terminal six histidine tag.

The SARS-CoV-2 RBD antigen was produced in the ExpiCHO-S cell line (ThermoFisher). The ExpiCHO-S cell line were cultured in ExpiCHO Expression medium (ThermoFisher) at 37° C., 80% humidity and 8% CO₂, with agitation at 125 rpm for expansion. Then, the cells at a density of 6×10⁶ cells/ml were transiently transfected with 1 μg/ml of expression plasmid pre-mixed with ExpiFectamine CHO Reagent (ThermoFisher) in OptiPRO SFM complexation medium (ThermoFisher). At 5 days of culture, the cells were removed by centrifugation at 300 g at room temperature for 5 minutes, and the supernatant was preserved.

The supernatant was then purified by immobilized metal-affinity chromatography with the 5 ml column HiScreen Ni FF (Cytiva). The target protein was eluted with buffer comprising 20 mM sodium phosphate, 500 mM NaCl, 500 mM imidazole at pH 7.2. The purified target protein was dyalized with PBS (10 kDa RC membrane), concentrated at 1 mg/ml and filtered using PES filter of 0.22 μm pore size (Millex-GP) and stored at −80° C. for further use.

For the expression of the RBD and S1 antigens of SARS-CoV-2 in the HEK293 cell line, the same method as described for the CHO cell expression was used. The components used for the HEK293 expression were the Expi293F (ThermoFisher) cell line, the Expi293 Expression medium (ThermoFisher), the ExpiFectamine 293 Reagent (ThermoFisher), and the Opti-MEM complexation medium (ThermoFisher).

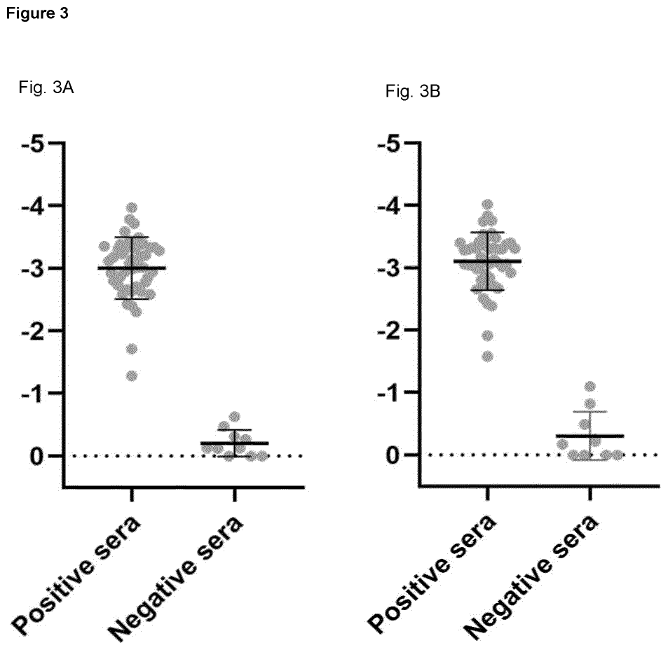

Two different serum collections were gathered: one commercially available from Ray Biotech (Ref. CoV-PosSet-S1), and one obtained from convalescent, hospitalized patients from the Girona region (Catalunya, Spain) with different levels of anti-SARS-CoV-2 antibodies. Thirty (30) serums, obtained from positive PCR-confirmed patients, were confronted in parallel with the candidate RBD antigen produced in either HEK293 or CHO. Moreover, 10 negative serum samples obtained before the pandemic outbreak were included in the study. Total human SARS-CoV-2 RBD IgG antibody titers were determined for each sample (log 10 EC₅₀), revealing that all convalescent serum samples had antibody levels against RBD clearly higher than the negative samples, regardless the origin of the antigen (FIG. 3). The consistent response against RBD in convalescent samples shows the significance of RBD as an antigen candidate for a COVID-19 vaccine.

The determination of total human SARS-CoV-2 RBD IgG antibody titers was performed by ELISA. Nunc Maxisorp ELISA plates (ThermoFisher, ref. 10547781) were coated with 100 ng per well of SARS-CoV-2 RBD protein (positions 319 to 541 of the SARS-CoV-2 Spike glycoprotein, UniProt No. P0DTC2) overnight at 4° C. Plates were washed with phosphate buffered saline with 0.05% Tween buffer and blocked with Stabilblock Immunoassay Stabilizer buffer (Surmodics IVD, ref. ST01-1000). Sera samples obtained from mice were 4-fold serially diluted and added to coated wells for 1 hours at 37° C., 5% CO₂ and humidified atmosphere. The plates were washed with PBS. Next, diluted horseradish peroxidase (HRP) conjugated with anti-mouse (Jackson ImmunoResearch, ref. 115-035-003) was added and color developed by addition of 2,2′-azino-di-(3-ethylbenzthiazoline sulfonic acid) peroxidase substrate (ABTS, CIVTEST). Plates were read at an OD of 405 nm with a Gene5 plate reader (Synergy HTX, multi-mode reader) and data analyzed with SoftMax software. The concentration of the antibody that gives half-maximal binding (EC₅₀ values) was calculated by 4-parameter fitting using GraphPad Prism software.

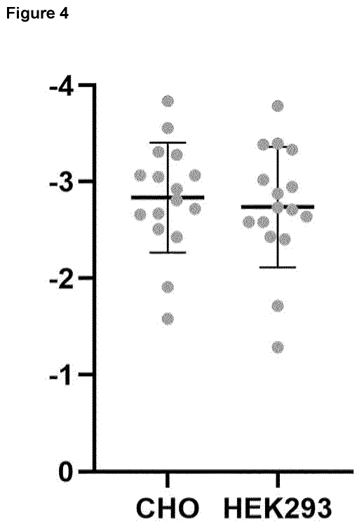

A high degree of equivalence was seen between both expression systems in terms of IgG antibody titers. Comparing the grouped IgG antibody titers against SARS-CoV-2 RBD produced in HEK293 cells or SARS-CoV-2 RBD produced in CHO cells, no significant difference was detected (FIG. 4). Moreover, paired IgG antibody titers against SARS-CoV-2 RBD produced in HEK293 cells and SARS-CoV-2 RBD produced in CHO cells for each single serum sample showed a very good similarity (FIG. 6). Together, these results support the equivalence between the production of the antigens in both mammalian expression systems.

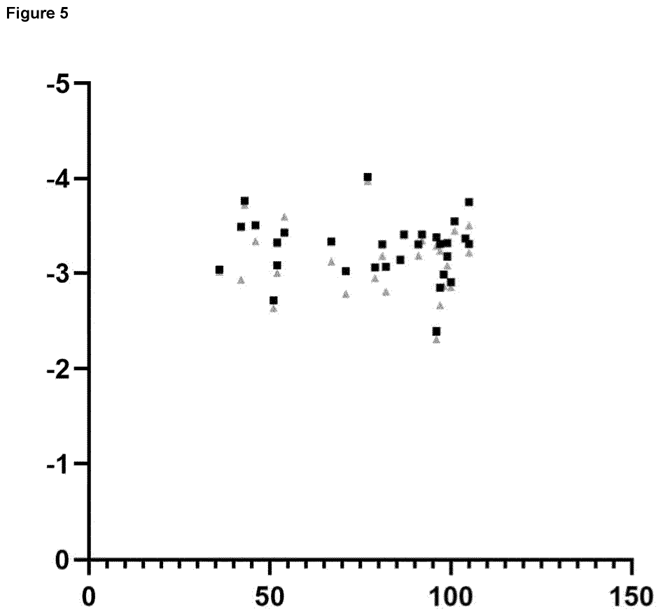

The correlation between IgG antibody titers against SARS-CoV-2 RBD and the elapsed days between the first PCR-positive result and each serum sample donation was plotted (FIG. 5). In the time period of the collection of samples (between 36 and 105 days), no clear correlation was seen between both factors. This result suggests that antibodies specifically raised against SARS-CoV-2 RBD in convalescent patients might last for several months at least.

### Example 4: Immunogenicity Study in Mice with Different Vaccine Candidates

This study evaluates different RBD subunit vaccine candidates against SARS-CoV-2. The study also evaluates different RBD subunit vaccine formulations. Besides, this study assesses the immunogenic capabilities of the different protein subunit vaccine candidates.

A total of 46 BALB/c mice of 5-6 weeks of age were selected for the study. Mice were divided into 5 different Groups which received a different vaccine formulation. Ten mice were allotted in each group, with the exception of the control Group (Group A) which included 6 mice. Animals received two doses of 0.1 ml of one of the following protein subunit vaccine formulations administered intramuscularly. The vaccines were formulated as a “ready-to-use” vaccine. The first dose (priming) was administered to the animals on Day 0 and the second dose (boosting) 18 days after the first dose (Day 18).

The different vaccine formulations administered to mice were the following:
Group A: control group. The animals in this group received a mock-vaccine comprising PBS.
Group B: animals in this group received a vaccine comprising 20 μg of recombinant RBD antigen of SARS-CoV-2. The recombinant antigen was based on a proportion of 12% of non-fusion dimeric RBD antigen and 88% of monomeric RBD antigen. The proportion of non-fusion dimer:monomer was determined by a size-exclusion chromatography HPLC (Agilent Technologies). The vaccine was formulated with an oil-in-water adjuvant formulated as 19.5 mg/ml of squalene, 2.35 mg/ml of polysorbate 80, 2.35 mg/ml of sorbitan trioleate, 1.32 mg/ml of sodium citrate and 0.08 mg/ml of citric acid, at a ratio v/v 75% adjuvant and 25% antigen.
Group C: animals in this group received a vaccine comprising 20 μg of recombinant RBD antigen of SARS-CoV-2. The recombinant antigen was based on a proportion of 12% of non-fusion dimeric RBD antigen and 88% of monomeric RBD antigen (the proportion of non-fusion dimer:monomer was determined as in Group B). The vaccine was formulated with the same adjuvant as in Group B at a ratio v/v 75% adjuvant and 25% antigen, together with 10 μg/dose of MPLA (L6895, SIGMA) which was added into the antigenic phase.
Group D: animals in this group received a vaccine comprising 20 μg of recombinant RBD antigen of SARS-CoV-2. The recombinant antigen was based on a proportion of 12% of non-fusion dimeric RBD antigen and 88% of monomeric RBD antigen (the proportion of non-fusion dimer:monomer was determined as in Group B). The vaccine was formulated with the same adjuvant as Group B at a ratio v/v 75% adjuvant and 25% antigen, together with 10 μg/dose of QS-21 (QS-21, DESERT KING) which was added into the antigenic phase.
Group E: animals in this group received a vaccine comprising 10 μg of recombinant RBD antigen of SARS-CoV-2. The recombinant RBD used in this group was adjusted to comprise a higher proportion of non-fusion dimeric RBD antigen. The recombinant antigen used in this group comprised 56% of non-fusion dimeric RBD antigen and 44% of monomeric RBD antigen. For the adjustment of the proportion of dimeric SARS-CoV-2 RBD antigen, the dimeric and monomeric RBD antigen fractions were separated by size-exclusion chromatography (HiPrep 26/60 Sephacryl S-100 HR, Cytiva ref. 17119401). The final antigen concentration of each fraction was determined with a microplate reader (Synergy HTX, multi-mode reader) read at an OD of 280 nm. Finally, the fractions comprising the monomeric RBD antigen and non-fusion dimeric RBD antigen were mixed at different volumes to obtain a specific proportion of dimeric RBD antigen. The vaccine was formulated with the same adjuvant as group B at a ratio v/v 75% adjuvant and 25% antigen.

The oil-in-water adjuvant used in this study as indicated above was formulated as follows: 19.5 mg/ml of squalene, 2.35 mg/ml of polysorbate 80, 2.35 mg/ml of sorbitan trioleate, 1.32 mg/ml of sodium citrate and 0.08 mg/ml of citric acid. Thus, each of the 0.1 ml dose of the administered vaccine, when mixed at a proportion of 75% adjuvant with 25% antigen, comprised 1.46 mg of squalene, 0.18 mg of polysorbate 80, 0.18 mg of sorbitan trioleate, 0.099 mg of sodium citrate and 0.006 mg of citric acid.

The method of preparing the oil-in-water adjuvant was the same as Example 1.

The recombinant RBD antigen was produced in the ExpiCHO-S cell line as follows: The SARS-CoV-2 RBD sequence for producing the RBD subunit antigen used in this study consisted of positions 319 to 541 of the SARS-CoV-2 Spike glycoprotein (UniProt No. P0DTC2). For the preparation of the vaccine, the RBD gene was codon-optimized for CHO-cell expression and further cloned into the expression plasmid pD2610-v10 (transient expression vector from ATUM) with an N-terminal signal peptide of sequence MGWSLILLFLVAVATRVLS (SEQ ID NO: 10) for transient expression, and a C-terminal six histidine tag.

The SARS-CoV-2 RBD antigen was produced in the ExpiCHO-S cell line (ThermoFisher). The ExpiCHO-S cell line were cultured in ExpiCHO Expression medium (ThermoFisher) at 37° C., 80% humidity and 8% CO₂, with agitation at 125 rpm for expansion. Then, the cells at a density of 6×10⁶ cells/ml were transiently transfected with 1 μg/ml of expression plasmid pre-mixed with ExpiFectamine CHO Reagent (ThermoFisher) in OptiPRO SFM complexation medium (ThermoFisher). At 5 days of culture, the cells were removed by centrifugation at 300 g at room temperature for 5 minutes, and the supernatant was preserved.

The supernatant was then purified by immobilized metal-affinity chromatography with the 5 ml column HiScreen Ni FF (Cytiva). The target protein was eluted with buffer comprising 20 mM sodium phosphate, 500 mM NaCl, 500 mM imidazole at pH 7.2. The purified target protein was dialyzed against PBS (10 kDa RC membrane), concentrated at 1 mg/ml and filtered using PES filter of 0.22 μm pore size (Millex-GP) and stored at −80° C. for further use.

To assess the immunological response against SARS-CoV-2 after the vaccination protocol, sera samples from each mice were extracted on Day 18 (FIG. 7A), prior to the second dose of the vaccine, and on Day 30 (FIG. 7B) of the study. The sera samples were analyzed for antibodies anti-SARS-CoV-2 RBD IgG titers by ELISA. The log 10 EC₅₀ titers of antibodies anti-SARS-CoV-2 RBD IgG per group are represented in FIG. 7.

Nunc Maxisorp ELISA plates (ThermoFisher, ref. 10547781) were coated with 100 ng per well of SARS-CoV-2 RBD protein (positions 319 to 541 of the SARS-CoV-2 Spike glycoprotein, UniProt No. P0DTC2) overnight at 4° C. Plates were washed with phosphate buffered saline with 0.05% Tween buffer and blocked with Stabilblock Immunoassay Stabilizer buffer (Surmodics IVD, ref. ST01-1000). Sera samples obtained from mice were 4-fold serially diluted and added to coated wells for 1 hours at 37° C., 5% CO₂ and humidified atmosphere. The plates were washed with PBS. Next, diluted horseradish peroxidase (HRP) conjugated with anti-mouse (Jackson ImmunoResearch, ref. 115-035-003) was added and color developed by addition of 2,2′-azino-di-(3-ethylbenzthiazoline sulfonic acid) peroxidase substrate (ABTS, CIVTEST). Plates were read at an OD of 405 nm with a Gene5 plate reader (Synergy HTX, multi-mode reader) and data analyzed with SoftMax software. EC₅₀ values were calculated by 4-parameter fitting using GraphPad Prism software.

Results clearly show that animals immunized with all the vaccine candidates following a two-dose regimen (Groups B to E) had significantly higher anti-SARS-CoV-2 RBD antibodies on day 30, in comparison to the control group (Group A). Therefore, the results show the capacity to generate an immune response of compositions comprising RBD antigen. The results also demonstrate the suitability of using the oil-in-water adjuvant in subunit SARS-CoV-2 vaccines comprising RBD antigen. Furthermore, it is clearly observed that a higher humoral response is obtained when combining the vaccine formulation with an immunostimulant, specifically MPLA (Group C). Surprisingly, animals in Group C achieved a high immune response on day 18 after the first dose. This group also had the highest titers of anti-SARS-CoV-2 RBD antibodies on day 30 as compared with the other Groups.

Results also demonstrate that when the RBD antigen present in the vaccine formulation has a high proportion of non-fusion dimeric RBD antigen over the monomeric RBD antigen (Group E), the humoral response is significantly increased, even if the vaccine composition does not comprise an immunostimulant. It was observed, that a vaccine comprising a half dose of RBD antigen (10 μg/dose) formulated at high proportion of non-fusion dimeric RBD antigen and with the oil-in-water adjuvant provided an increased humoral response on day 30 (Group E) when compared to the group that received a formulation vaccine comprising a low proportion of non-fusion dimeric RBD antigen without immunostimulant (Group B), and with immunostimulant QS-21 (Group D). It was also observed that the vaccine administered to animals in Group E, which comprised 10 μg of recombinant RBD of SARS-CoV-2 antigen with an increased proportion of non-fusion dimeric RBD antigen and without immunostimulant, resulted in equivalent anti-SARS-CoV-2 RBD antibody titers to the ones obtained in the animals in Group C, which received a vaccine comprising 20 μg of recombinant RBD of SARS-CoV-2 antigen with a reduced proportion of non-fusion dimeric RBD antigen over monomeric RBD antigen together with MPLA as immunostimulant.

The results unexpectedly showed the strong capacity to generate anti-SARS-CoV-2 RBD antibodies of formulations based on the non-fusion dimeric RBD antigen and of formulations based on the RBD antigen with an increased proportion of non-fusion dimeric RBD antigen over monomeric RBD antigen.

### Example 5: Immunogenicity Study in Mice with Fusion Dimeric RBD Antigen

This study evaluates a novel recombinant subunit antigen of SARS-CoV-2. The novel recombinant subunit antigen is a fusion dimeric RBD antigen that contains two monomers, a first monomer comprising a RBD derived from the B.1.351 (South Africa) variant and a second monomer comprising a RBD derived from the B.1.1.7 (UK) variant. This novel recombinant subunit antigen of SARS-CoV-2 is named herein as fusion dimeric RBD variant antigen. The study evaluates different subunit vaccine formulations and the immunogenic capabilities in mice of this recombinant fusion dimeric RBD variant antigen. The present study further includes a comparison between the fusion dimeric RBD variant antigen and the previously described recombinant non-fusion dimeric:monomeric RBD antigen of SARS-CoV-2 that consists of a combination of non-fusion dimeric RBD antigen and monomeric RBD antigens derived from Wuhan variant and formulated in different dimer:monomer proportions. This latter protein subunit vaccine is named herein as non-fusion dimeric:monomeric RBD non-variant SARS-CoV-2 antigen.

The recombinant fusion dimeric RBD variant SARS-CoV-2 antigen used in this study is a tandem that comprises as the first monomer the amino acid sequence of positions 319 to 537 of SARS-CoV-2 Spike protein RBD monomer derived from the B.1.351 variant, as defined in SEQ ID NO: 4, followed by the amino acid sequence of positions 319 to 537 of SARS-CoV-2 Spike protein RBD monomer derived from the B.1.1.7 variant, as defined in SEQ ID NO: 3, as the second monomer. The amino acid sequence of this recombinant fusion dimeric RBD variant antigen as a tandem fusion antigen is defined in SEQ ID NO: 5.

For the preparation of the vaccine, the fusion dimeric RBD variant SARS-CoV-2 antigen was codon-optimized for CHO-cell expression (SEQ ID NO: 8) and further cloned into the expression plasmid pcDNA3.4 (GENSCRIPT) with an N-terminal signal peptide of sequence MGWSCIILFLVATATGVHS (SEQ ID NO: 6) for transient expression, and a C-terminal six histidine tag. The DNA construct comprising the signal peptide, the codon-optimized SARS-CoV-2 RBD dimeric variant and the C-terminal histidine tag is defined in SEQ ID NO: 7.

The recombinant non-fusion dimeric:monomeric RBD non-variant SARS-CoV-2 antigen used in this study consisted of positions 319 to 541 of the SARS-CoV-2 Spike glycoprotein of Wuhan-Hu-1 variant (UniProt No. P0DTC2). To produce the recombinant non-fusion dimeric:monomeric RBD non-variant SARS-CoV-2 antigen, the RBD gene was codon-optimized for CHO-cell expression and further cloned into the expression plasmid pD2610-v10 (ATUM) with an N-terminal signal peptide of sequence MGWSLILLFLVAVATRVLS (SEQ ID NO: 10) for transient expression, and a C-terminal six histidine tag.

The recombinant fusion dimeric RBD variant SARS-CoV-2 antigen and the recombinant non-fusion dimeric:monomeric RBD non-variant SARS-CoV-2 antigen were produced in the ExpiCHO-S cell line (ThermoFisher). The ExpiCHO-S cell line were cultured in ExpiCHO Expression medium (ThermoFisher) at 37° C., 80% humidity and 8% CO₂, with agitation at 125 rpm for expansion. Then, the cells at a density of 6×10⁶ cells/ml were transiently transfected with 1 μg/ml of expression plasmid pre-mixed with ExpiFectamine CHO Reagent (ThermoFisher) in OptiPRO SFM complexation medium (ThermoFisher). At 5 days of culture, the cells were removed by centrifugation at 300 g at room temperature for 5 minutes, and the supernatant was preserved.

The supernatant was then purified by immobilized metal-affinity chromatography with the 5 ml column HiScreen Ni FF (Cytiva). The target protein was eluted with buffer comprising 20 mM sodium phosphate, 500 mM NaCl, 500 mM imidazole at pH 7.2. The purified target protein was dialyzed against PBS-0.01% Tween 80 (30 kDa RC membrane), concentrated at 1 mg/ml and filtered using PES filter of 0.22 μm pore size (Millex-GP) and stored at −80° C. for further use.

A total of 86 BALB/c mice of 5-6 weeks of age were selected for the study. Mice were allotted into 9 different Groups. Each group received a different vaccine formulation as described below. Each Group included 10 mice with the exception of the control Group (Group A) which only included 6 mice. All animals were administered with one dose of 0.1 ml of the following vaccine formulations by intramuscular route. The vaccines were formulated as a “ready-to-use” vaccine. The vaccine was administered to the animals on the Day 0 of the study.

The different vaccine formulations administered to mice were the following:
Group A: Control group. The animals in this Group received a mock-vaccine comprising PBS.
Group B (fusion dimeric RBD variant SARS-CoV-2 antigen at 1× dose): animals in this group received a vaccine formulation comprising 1 μg of the recombinant fusion dimeric RBD variant SARS-CoV-2 antigen. The vaccine was formulated with an oil-in-water adjuvant at a ratio v/v 50% adjuvant and 50% antigen.
Group C (fusion dimeric RBD variant SARS-CoV-2 antigen at 5× dose): animals in this group received a vaccine formulation comprising 5 μg of the recombinant fusion dimeric RBD variant SARS-CoV-2 antigen. The vaccine was formulated with an oil-in-water adjuvant at a ratio v/v 50% adjuvant and 50% antigen
Group D (fusion dimeric RBD variant SARS-CoV-2 antigen at 20× dose): animals in this group received a vaccine formulation comprising 20 μg of the recombinant fusion dimeric RBD variant SARS-CoV-2 antigen. The vaccine was formulated with an oil-in-water adjuvant at a ratio v/v 50% adjuvant and 50% antigen.
Group E (fusion dimeric RBD variant SARS-CoV-2 antigen at 20× dose plus immunostimulant 1): animals in this group received a vaccine formulation comprising 20 μg of the recombinant fusion dimeric RBD variant SARS-CoV-2 antigen. The vaccine was formulated with an oil-in-water adjuvant at a ratio v/v 50% adjuvant and 50% antigen, together with 10 μg/dose of MPLA (L6895, SIGMA) which was added in the antigenic phase.
Group F (fusion dimeric RBD variant SARS-CoV-2 antigen at 20× dose plus immunostimulant 2): animals in this group received a vaccine formulation comprising 20 μg of the recombinant fusion dimeric RBD variant SARS-CoV-2 antigen. The vaccine was formulated with an oil-in-water adjuvant at a ratio v/v 50% adjuvant and 50% antigen, together with 10 μg/dose of QS-21 (QS-21, DESERT KING) which was added in the antigenic phase.
Group G (non-fusion dimeric:monomeric RBD non-variant SARS-CoV-2 antigen at 20× dose): animals in this group received a vaccine formulation comprising 20 μg of the recombinant non-fusion dimeric:monomeric RBD non-variant SARS-CoV-2 antigen. This recombinant antigen was based on the recombinant SARS-CoV-2 RBD antigen in a non-fusion dimeric form at a proportion of 80%:20% non-fusion RBD dimer:RBD monomer. For the adjustment of the proportion of non-fusion dimeric SARS-CoV-2 RBD antigen, the non-fusion dimeric and monomeric RBD antigen fractions were separated by size-exclusion chromatography (HiPrep 26/60 Sephacryl S-100 HR, Cytiva ref. 17119401). The final antigen concentration of each fraction was determined with a microplate reader (Synergy HTX, multi-mode reader) read at an OD of 280 nm. Finally, the fractions comprising the monomeric RBD antigen and the non-fusion dimeric RBD antigen were mixed at different volumes to obtain a specific proportion of non-fusion dimeric RBD antigen. The vaccine was formulated with an oil-in-water adjuvant at a ratio v/v 50% adjuvant and 50% antigen.
Group H (non-fusion dimeric:monomeric RBD non-variant SARS-CoV-2 antigen at 20× dose with immunostimulant 1): animals in this group received a vaccine formulation comprising 20 μg of the non-fusion dimeric:monomeric RBD non-variant SARS-CoV-2 antigen. The recombinant antigen was based on the recombinant SARS-CoV-2 RBD antigen in a non-fusion dimeric form at a proportion of 80%:20% non-fusion RBD dimer:RBD monomer (the adjustment of the proportion of dimer:monomer was performed as in Group G). The vaccine was formulated with an oil-in-water adjuvant at a ratio v/v 50% adjuvant and 50% antigen, together with 10 μg/dose of MPLA (L6895, SIGMA) which was added in the antigenic phase.
Group I (non-fusion dimeric:monomeric RBD non-variant SARS-CoV-2 antigen at 20× dose with immunostimulant 2): animals in this group received a vaccine formulation comprising 20 μg of the non-fusion dimeric:monomeric RBD non-variant SARS-CoV-2 antigen. The recombinant antigen was based on the recombinant SARS-CoV-2 RBD antigen in a non-fusion dimeric form at a proportion of 80%:20% non-fusion RBD dimer:RBD monomer (the adjustment of the proportion of dimer:monomer was performed as in Group G). The vaccine was formulated with an oil-in-water adjuvant, at a ratio v/v 50% adjuvant and 50% antigen, together with 10 μg/dose of QS-21 (QS-21, DESERT KING) which was added in the antigenic phase.

The oil-in-water adjuvant used in this study was formulated as follows: 39 mg/ml of squalene, 4.7 mg/ml of polysorbate 80, 4.7 mg/ml of sorbitan trioleate, 2.64 mg/ml of sodium citrate, and 0.16 mg/ml of citric acid. Thus, the 0.1 ml dose of the administered vaccine, when mixed at proportion of 50% adjuvant with 50% antigen, comprises 1.95 mg of squalene, 0.235 mg of polysorbate 80, 0.235 mg of sorbitan trioleate, 0.132 mg of sodium citrate and 0.008 mg of citric acid. This formulation is analogous to the standard concentration of known oil-in-water adjuvants administered in humans in a dose of 0.5 ml which is 9.75 mg of squalene, 1.175 mg of polysorbate 80, 1.175 mg of sorbitan trioleate, 0.66 mg of sodium citrate and 0.04 mg of citric acid.

To assess the immunological response against SARS-CoV-2 after vaccination with the different vaccine formulations of the study, sera samples from each mice were extracted on Day 21 and were analyzed for anti-SARS-CoV-2 RBD IgG antibody titers by ELISA. The log 10 EC₅₀ values of the titers of anti-SARS-CoV-2 RBD IgG antibodies are represented in FIG. 8.

The determination of anti-SARS-CoV-2 IgG antibody titers was performed by ELISA as follow: Nunc Maxisorp ELISA plates (ThermoFisher, ref. 10547781) were coated with 100 ng per well of SARS-CoV-2 RBD protein overnight at 4° C. For the analysis of sera extracted from animals of Groups A to F the ELISA plates were coated with the recombinant fusion dimeric RBD variant SARS-CoV-2 antigen, as described above, and for the analysis of sera extracted form animals of Groups G to I the ELISA plates were coated with the recombinant non-fusion 80% dimeric:20% monomeric RBD non-variant SARS-CoV-2 RBD antigen, as described above. Plates were washed with phosphate buffered saline with 0.05% Tween buffer and blocked with Stabilblock Immunoassay Stabilizer buffer (Surmodics IVD, ref. ST01-1000). Sera samples obtained from mice were 4-fold serially diluted and added to coated wells for 1 hours at 37° C., 5% CO2 and humidified atmosphere. The plates were washed with PBS. Next, diluted horseradish peroxidase (HRP) conjugated with anti-mouse (Jackson ImmunoResearch, ref. 115-035-003) was added and color developed by addition of 2,2′-azino-di-(3-ethylbenzthiazoline sulfonic acid) peroxidase substrate (ABTS, CIVTEST). Plates were read at an OD of 405 nm with a Gene5 plate reader (Synergy HTX, multi-mode reader) and data analyzed with SoftMax software. EC₅₀ values were calculated by 4-parameter fitting using GraphPad Prism software.

From the results of the study, it was surprisingly observed that after administering one single dose of the recombinant fusion dimeric RBD variant SARS-CoV-2 antigen the vaccinated animals presented an increased immune response. Even animals which received the vaccine formulation comprising a low dose of the fusion dimeric RBD variant SARS-CoV-2 antigen without any immunostimulant (Groups B and C), produced higher anti-SARS-CoV-2 RBD IgG antibody titers than groups that received a vaccine formulation comprising 20 μg dose of the non-fusion dimeric:monomeric RBD non-variant SARS-CoV-2 antigen with an oil-in-water adjuvant alone (Group G) or with an oil-in-water adjuvant further including QS-21 immunostimulant (Group I). At equal doses of total antigen present in the vaccine composition, such as 20 μg, it was also observed an increased response for those groups that received the recombinant fusion dimeric RBD variant SARS-CoV-2 antigen (Groups D-F) even if the composition did not comprise any immunostimulant (Group D) when compared to the groups that received vaccine compositions comprising the non-fusion dimeric:monomeric RBD non-variant SARS-CoV-2 antigen formulated with an oil-in-water adjuvant and MPLA immunostimulant (Group H). Overall, the results unexpectedly indicated the increased potential to generate anti-SARS-CoV-2 RBD IgG antibodies against SARS-CoV-2 of recombinant dimeric RBD antigens (fusion and non-fusion dimeric RBD).

Furthermore, results confirm the suitability of using the oil-in-water adjuvant formulated as 39 mg/ml of squalene, 4.7 mg/ml of polysorbate 80, 4.7 mg/ml of sorbitan trioleate, 2.64 mg/ml of sodium citrate, and 0.16 mg/ml of citric acid in the final vaccine formulation. Finally, animals vaccinated with the vaccine compositions comprising the oil-in-water adjuvant combined with an immunostimulant, particularly with MPLA, showed higher antibody titers than animals immunized with the oil-in-water adjuvant without an immunostimulant, indicating a better immune response of the vaccinated subjects when they received both, an adjuvant and an immunostimulant with the fusion dimeric RBD variant antigen or the non-fusion dimeric:monomeric RBD non-variant SARS-CoV-2 antigen.

### Example 6: Immunogenicity Study in Mice with Two Doses of a Fusion Dimeric RBD Antigen

This study evaluates the novel recombinant fusion dimeric RBD variant SARS-CoV-2 antigens in a two-dose regimen, considering the unexpected good results obtained after vaccinating animals with a one dose vaccine comprising fusion dimeric RBD variant SARS-CoV-2 antigens, as described in Example 5.

In this study, the animals pertaining to the different Groups described in Example 5, received a second dose of the vaccine. On Day 21, 21 days after the first dose, animals received a second dose of 0.1 ml of the corresponding vaccine formulation per group, Groups A to I as described in Example 5, by intramuscular route.

To assess the immunological response against SARS-CoV-2 after the second dose of the different vaccine formulations of the study, sera samples from each mice were extracted on Day 35, 14 days after the second dose, and were analyzed for anti-SARS-CoV-2 RBD IgG antibody titers by ELISA, as described in Example 5. The log 10 EC₅₀ values of the titers of anti-SARS-CoV-2 RBD IgG antibodies present in the sera of animals 14 days after the second dose are represented in FIG. 9.

After a second dose of the protein subunit vaccines, an increase in the anti-SARS-CoV-2 RBD IgG antibody titers is observed in all vaccinated groups compared to the anti-SARS-CoV-2 RBD IgG antibody titers after a single dose. Therefore, a prime/boost or two dose protocol increases the immunogenic response. Furthermore, the animals that received a vaccine comprising the fusion dimeric RBD variant SARS-CoV-2 antigen presented an increase in the anti-SARS-CoV-2 RBD IgG antibody titers (Groups B to F) compared to the animals that received a vaccine comprising non-fusion dimeric:monomeric RBD non-variant SARS-CoV-2 antigen (Groups G to I). Even the animals that received a vaccine formulated at low doses of fusion dimeric RBD variant SARS-CoV-2 antigen and without any immunostimulant (Groups B and C) presented an increase in the anti-SARS-CoV-2 RBD IgG antibody titers compared to the animals that received a vaccine comprising non-fusion dimeric:monomeric RBD non-variant SARS-CoV-2 antigen (Groups G to I). Overall, these results confirm the unexpected potential to generate anti-SARS-CoV-2 IgG antibodies of the dimeric RBD antigens, particularly of the fusion dimeric RBD variant SARS-CoV-2 antigen, that were obtained in Example 5.

Form the results it can be concluded that the protein subunit vaccine, particularly based on a dimeric RBD antigens would be also suitable to be used as a booster vaccine in combination with other vaccines or in annual revaccinations, as it increased significantly the anti-SARS-CoV-2 RBD IgG antibody titers.

### Example 7: Immunogenicity Study in Mice with a Non-Fusion Dimeric RBD Antigen Compared to a Commercially Available Vaccine

This study evaluates the immunogenicity of a subunit vaccine candidate based on a non-variant SARS-CoV-2 RBD antigen formulated to comprise a high proportion of non-fusion dimeric RBD antigen over monomeric RBD antigen. The vaccine candidate was formulated with an oil-in-water adjuvant formulated as 39 mg/ml of squalene, 4.7 mg/ml of polysorbate 80, 4.7 mg/ml of sorbitan trioleate, 2.64 mg/ml of sodium citrate, and 0.16 mg/ml of citric acid, with and without MPLA as immunostimulant and immunogenicity was compared with a commercially available SARS-CoV-2 vaccine Spikevax, COVID-19 mRNA Vaccine (Moderna Biotech Spain, S.L.).

A total of 46 BALB/c mice of 6-7-weeks-old were distributed in 4 different Groups. Each Group received a different vaccine formulation as described below. Animals received two doses of 0.1 ml of the vaccine by intramuscular route three-weeks apart, the first dose was administered on Day 0 (priming) and a second dose (booster) was administered on Day 21.
Group A (control group, 10 animals): Animals in this group received a mock-vaccine comprising PBS.
Group B (non-fusion dimeric:monomeric RBD non-variant SARS-CoV-2 antigen, 12 animals): Animals in this group received a vaccine formulation comprising 20 μg of the recombinant non-fusion dimeric:monomeric RBD non-variant antigen at a proportion 80%:20% non-fusion RBD dimer:RBD monomer. The preparation of the recombinant non-fusion dimeric:monomeric RBD non-variant SARS-CoV-2 antigen and the proportion of the dimer:monomer of the non-fusion dimeric SARS-CoV-2 RBD antigen was carried out as described in Example 5. The vaccine was formulated with an oil-in-water adjuvant at a ratio v/v 50% adjuvant and 50% antigen. Thus, a 0.1 ml dose of the vaccine, when mixed at proportion of 50% adjuvant and 50% antigen, comprises 1.95 mg of squalene, 0.235 mg of polysorbate 80, 0.235 mg of sorbitan trioleate, 0.132 mg of sodium citrate and 0.008 mg of citric acid.
Group C (non-fusion dimeric:monomeric RBD non-variant SARS-CoV-2 antigen with an immunostimulant, 12 animals): Animals in this group received a vaccine formulation comprising 20 μg of the recombinant non-fusion dimeric:monomeric RBD non-variant antigen at a proportion 80%:20% non-fusion RBD dimer:RBD monomer (preparation of the antigen and the proportion of dimer:monomer used in this study was carried out as described in Example 5). The vaccine was formulated with the same adjuvant as Group B at a ratio v/v 50% adjuvant and 50% antigen, together with 10 μg/dose of MPLA as immunostimulant (L6895, SIGMA) which was added in the antigenic phase.
Group D (commercial vaccine, 12 animals): Animals in this group received a vaccine formulation comprising 1 μg mRNA (embedded in SM-102 lipid nanoparticles) of the Spikevax vaccine, COVID-19 mRNA Vaccine (Moderna Biotech Spain, S.L.). The doses of the commercial vaccine administered to mice in this study were obtained from well-preserved residual volumes of vials provided by public health institutions after vaccinating human population. The dose (1 μg mRNA) was chosen based on the paper Corbett K.S., et al. SARS-CoV-2 mRNA vaccine design enabled by prototype pathogen preparedness. Nature, 2020, vol. 586, no 7830, p. 567-571, where it is shown in a dose response study that this dose is near to the saturation limit of detection in mice.

To assess the immunogenicity response against SARS-CoV-2 after vaccination with the different vaccine formulations of the study, sera samples from all animals were extracted on day 21 (before the second dose) and on day 35 (14 days after the second dose) and they were analysed for anti-SARS-CoV-2 RBD IgG antibody titres by ELISA. Furthermore, the sera samples extracted on day 35 were analysed for determining neutralizing antibodies against SARS-CoV-2 isolate Wuhan-1 (Wuhan-Hu-1) by a pseudovirus-based neutralization assay (PBNA). Due to laboratory limitations some samples were not possible to be analysed on day so half of the sera samples were finally extracted and tested on day 37 for both anti-SARS-CoV-2 RBD IgG titres and neutralizing antibodies against SARS-CoV-2.

The determination of anti-SARS-CoV-2 RBD IgG antibody titres was performed by ELISA as follows:

Nunc Maxisorp ELISA plates (ThermoFisher, ref. 10547781) were coated with 100 ng per well of the recombinant SARS-CoV-2 RBD (Sino Biologicals, ref. 40592-V08B) and blocked with 5% non-fat dry milk (Sigma) in PBS. Plates were washed with phosphate buffered saline with 0.05% Tween buffer and blocked with Stabilblock Immunoassay Stabilizer buffer (Surmodics IVD, ref. ST01-1000). Wells were incubated with serial dilutions of the serum samples obtained from mice for 1 hour at 37° C., 5% C02 and humidified atmosphere. Then, the plates were washed with PBS. Next, peroxidase-conjugated goat anti-mouse IgG (Sigma, ref. AP308P) was added. Finally, wells were incubated with K-Blue Advanced Substrate (Neogen, ref. 379175) and the absorbance at 450 nm was measured using a Gene5 plate reader (Synergy HTX, multi-mode reader) and data analysed with SoftMax software. The mean value of the absorbance was calculated for each dilution of the serum sample run in duplicate. The end-point titre of SARS-CoV-2 RBD-specific total IgG binding antibodies was established as the reciprocal of the last serum dilution that gave 3 times the mean optical density of the negative control of the technique (wells without serum added).

The PBNA assay is based on the use of the HIV reporter pseudovirus that express the S protein of SARS-CoV-2, and the generation of luciferase, as described in Nie J. et al. Quantification of SARS-CoV-2 neutralizing antibody by a pseudotyped virus-based assay. Nat Protoc. 2020 November; 15(11):3699-563715. One HIV reporter pseudovirus expressing SARS-CoV-2 S protein from Wuhan-1 (Wuhan-Hu-1) and Luciferase was generated.

For neutralization assay, 200 TCID₅₀ of pseudovirus supernatant was preincubated with serial dilutions of the heat-inactivated serum samples for 1 h at 37° C. and then added onto ACE2 overexpressing HEK293T cells. After 48 h, cells were lysed with Britelite Plus Luciferase reagent (Perkin Elmer, Waltham, MA, USA). Luminescence was measured for 0·2 s with an EnSight Multimode Plate Reader (Perkin Elmer). Neutralization capacity of the serum samples was calculated by comparing the experimental RLU calculated from infected cells treated with each serum to the max RLUs (maximal infectivity calculated from untreated infected cells) and min RLUs (minimal infectivity calculated from uninfected cells), and expressed as percent neutralization:

% Neutralization=(RLUmax−RLUexperimental)/(RLUmax−RLUmin)*100.

Normalized dose response neutralization curves were fitted to a four-parameter curve with a variable slope using Graph Pad Prism (v8.3.0). All IC₅₀ values are expressed as reciprocal dilution (concentration required to inhibit 50% of infection).

From the results of the study, it was observed that vaccinated Groups B to D induced significant higher anti-SARS-CoV-2 RBD IgG antibody response compared to non-vaccinated control group (Group A). Furthermore, vaccine formulations administered to Groups C and D induced similar antibody titres on day 21. It was also observed that after receiving the second dose all vaccinated groups (including Group B without MPLA) induced similar antibody titres between Days 35 and 37 (14-16 days after receiving the booster). Notably, the vaccinated group C showed a tendency towards higher IgG antibody responses in comparison with group D (FIG. 10B)

This results confirm the ability of the non-fusion RBD dimeric:monomeric non-variant SARS-CoV-2 antigen, formulated with a high proportion of dimeric RBD as antigen, to generate an immune response against severe acute respiratory syndrome coronavirus 2 (SARS-CoV-2) confirming their suitability for preparing a vaccine against SARS-CoV-2 infections.

Furthermore, results of the neutralizing antibody response obtained with the different vaccine formulations of this study demonstrate that after the second dose an equivalent neutralizing antibody levels were obtained for all vaccinated groups (Groups B to D), demonstrating again the suitability of the non-fusion RBD dimeric:monomeric non-variant SARS-CoV-2 antigen, with a high proportion of dimeric RBD as antigen, for preparing vaccine compositions against SARS-CoV-2 infections. It was further observed that adding an immunostimulant to the vaccine formulation, such as MPLA, in Group C, provides a positive effect on the capacity of generating neutralizing antibody titres (FIG. 11), confirming the tendency mentioned in FIG. 10B above.

### Example 8: Seroneutralization Assay of a Fusion Dimeric RBD Antigen Against SARS-CoV-2 Variants

This study evaluates the neutralization capacity of the novel recombinant fusion dimeric RBD variant SARS-CoV-2 antigen based on a first monomer comprising a RBD derived from the B.1. 351 (South Africa) variant and a second monomer comprising a RBD derived from the B.1.1.7 (UK) variant, against different SARS-CoV-2 variants of concern. This novel recombinant subunit antigen of SARS-CoV-2 is named fusion dimeric RBD variant antigen. The recombinant fusion dimeric RBD variant SARS-CoV-2 antigen is the same as described in Example 5, Groups B to F.

Groups D, E and F of Examples 5 and 6 were further selected for carrying out this study. These groups include the BALB/c mice of 5-6 weeks of age that were vaccinated with a dose of 20 μg of the fusion dimeric RBD variant SARS-CoV-2 antigen. Group D was formulated with the oil-in-water adjuvant used in Example 5, Group E was formulated with the same adjuvant as Group D together with 10 μg/dose of MPLA as immunostimulant, and Group F was formulated with the same adjuvant as Group D plus 10 μg/dose of QS-21 as immunostimulant, according to what it is described in Example 5.

In this study, sera samples from each mice in Groups D, E and F of Example 5 and 6 were extracted on day 45 (24 days after receiving the second dose) and analysed for neutralization capacity against different SARS-CoV-2 variants: Wuhan (Wuhan-Hu-1), U.K. (alpha; B.1.1.7), South Africa (beta; B.1.351), Brazil (gamma; P.1), and India (delta; B.1.617.2) variant.

Neutralizing antibodies in serum against SARS-CoV-2 Wuhan isolate (Wuhan-Hu-1), U.K. (alpha; B.1.1.7), South Africa (beta; B.1.351), Brazil (gamma; P.1) and India (delta; B.1.617.2) variants were determined by pseudovirus-based neutralization assay (PBNA). The neutralizing antibodies in serum against India variant (delta; B.1.617.2) were only determined for Group D (without immunostimulants).

Neutralizing antibodies in sera were determined by pseudovirus neutralization assay (PBNA) using a SARS-CoV-2 pseudovirus, as described in Nie J. et al. Quantification of SARS-CoV-2 neutralizing antibody by a pseudotyped virus-based assay. Nat Protoc. 2020 November; 15(11):3699-563715. Five pseudovirus expressing SARS-CoV-2 S protein, and luciferase were generated for this assay, each expressing the corresponding SARS-CoV-2 S protein of a different variant, namely Wuhan (Wuhan-Hu-1) isolate, U.K. (alpha; B.1.1.7) variant, South Africa (beta; B.1.351) variant, Brazil (gamma; P1) variant, and India (delta; B.1.617.2) variant. The difference in the Spike protein between the variants are known and well defined by the Centers for Disease Control and Prevention (CDC) “SARS-CoV-2 Variant Classifications and Definitions”.

For neutralization assay, 200 TCID₅₀ of each variant pseudovirus supernatant was preincubated with serial dilutions of the heat-inactivated serum samples of Groups D, E and F for 1 h at 37° C. and then added onto ACE2 overexpressing HEK293T cells. After 48 h, cells were lysed with Britelite Plus Luciferase reagent (Perkin Elmer, Waltham, MA, USA). Luminescence was measured for 0-2 s with an EnSight Multimode Plate Reader (Perkin Elmer). Neutralization capacity of the serum samples was calculated by comparing the experimental RLU calculated from infected cells treated with each serum to the max RLUs (maximal infectivity calculated from untreated infected cells) and min RLUs (minimal infectivity calculated from uninfected cells), and expressed as percent neutralization:

% Neutralization=(RLUmax−RLUexperimental)/(RLUmax−RLUmin)*100.

Normalized dose response neutralization curves were fitted to a four-parameter curve with a variable slope using Graph Pad Prism (v8.3.0). All IC₅₀ values are expressed as reciprocal dilution (concentration required to inhibit 50% of infection).

The results of this study surprisingly show that the immunization of animals with the recombinant fusion dimeric RBD SARS-CoV-2 antigen elicited comparable pseudovirus-neutralizing antibody titres against four different SARS-CoV-2 variants, such as Wuhan, U.K., South Africa and Brazil variants in all the groups. No significant differences between them were observed (FIG. 12). This demonstrates that the neutralizing antibody titters generated from vaccinating mice with the fusion dimeric RBD antigen are maintained at high levels regardless of the variant tested and the presence or absence of an immunostimulant such as of MPLA (Group E) or QS.21 (Group F) in the vaccine formulation.

Regarding pseudovirus-neutralizing antibody titers obtained against the India variant (delta) in Group D, the results shown in FIG. 12A also indicate that high levels of neutralizing antibody titers were also generated against this variant.

Altogether, the results show that the recombinant fusion dimeric RBD variant SARS-CoV-2 antigen is able to induce similar levels of antibody response without the need of an immunostimulant. Thus, demonstrating again the increased potential of the recombinant fusion dimeric RBD variant SARS-CoV-2 antigen in inducing an immune response, as already shown in previous Examples.

Overall, the results confirm that vaccine compositions based on the novel recombinant fusion dimeric RBD variant SARS-CoV-2 antigen induce high levels of immune response against different SARS-CoV-2 variants, including the new delta variant.

### Example 9: Safety and Immunogenicity Study in Pigs with a Fusion Dimeric RBD Antigen Against Different SARS-CoV-2 Variants Compared to a Commercially Available Vaccine

This study evaluates a novel recombinant subunit antigen of SARS-CoV-2. The novel recombinant subunit antigen is a fusion dimeric RBD antigen that contains two monomers, a first monomer comprising a RBD derived from the B.1.351 (South Africa) variant and a second monomer comprising a RBD derived from the B.1.1.7 (UK) variant. This novel recombinant subunit antigen of SARS-CoV-2 is named fusion dimeric RBD variant antigen. The recombinant fusion dimeric RBD variant SARS-CoV-2 antigen is the same as described in Example 5, Groups B to F.

The study evaluates the immunogenicity and safety in pigs of this recombinant fusion dimeric RBD variant antigen. Pigs have shown to be an animal model more suitable than small animal models to accurately predict vaccine outcome in humans.

A total of 13 large white-landrace cross-breeding pigs of 8-9 weeks of age were allocated in 3 different Groups. Each group received a different vaccine formulation as descried below.

Group A included 5 pigs and Groups B and C included 4 pigs each one. Animals received two doses separated 21 days apart, on Day 0 and Day 21. Each animal received 0.5 ml of the following vaccine formulations by intramuscular route per dose.

The different vaccine formulations administered to pigs were the following:
Group A (fusion dimeric RBD variant SARS-CoV-2 antigen): animals in this group received a vaccine formulation comprising 20 μg of the recombinant fusion dimeric RBD variant SARS-CoV-2 antigen. The vaccine was formulated with an oil-in-water adjuvant as 39 mg/ml of squalene, 4.7 mg/ml of polysorbate 80, 4.7 mg/ml of sorbitan trioleate, 2.64 mg/ml of sodium citrate, and 0.16 mg/ml of citric acid at a ratio v/v 50% adjuvant and 50% antigen. Thus, a 0.5 ml dose of the vaccine, when mixed at a proportion of 50% adjuvant and 50% antigen, comprises 9.75 mg of squalene, 1.175 mg of polysorbate 80, 1.175 mg of sorbitan trioleate, 0.66 mg of sodium citrate and 0.04 mg of citric acid.
Group B (commercial vaccine): animals in this group received the commercially available vaccine Spikevax, COVID-19 mRNA vaccine (Moderna Biotech Spain, S.L.). Spikevax comprises 100 μg of mRNA encoding the viral spike protein of SARS-CoV-2 (embedded in SM-102 lipid nanoparticles) per dose of 0.5 ml. The doses of the commercial vaccine administered to pigs in this study were obtained from well-preserved residual volumes of vials provided by public health institutions after vaccinating human population.
Group C (control group): animals in this group received a mock-vaccine comprising PBS.

To assess the safety profile after vaccination with the different vaccine formulations of the study, rectal temperatures were recorded one day before vaccination of each dose (on Day −1 and Day 20), at vaccination (Day 0), 4 and 6 hours post-vaccination and daily for three days after first and second vaccination. The mean rectal temperature per group was calculated (FIG. 13).

After receiving the first administration, animals in Group B (commercial vaccine) showed an average temperature increase that were considered abnormally higher at 6 hours after vaccination, although animals recovered one day later. Statistically significant differences were observed at 6 h post-vaccination between groups A (fusion dimeric RBD variant SARS-CoV-2 antigen) and B (commercial vaccine) after receiving the first dose. Mean temperatures in Group B were considered abnormally higher and with clinical affectation in the normal status of the animals (FIG. 13). Similar average temperature increase results are observed after the second dose. On the contrary, mean temperatures in Groups A (fusion dimeric RBD variant SARS-CoV-2 antigen) and C (control group) were considered within basal values during all study and no clinically relevant differences were observed between both groups. Thus, the results of this study demonstrate a better safety profile of a subunit vaccine based on fusion dimeric RBD variant SARS-CoV-2 antigen over a mRNA vaccine (FIG. 13).

To assess the immunogenicity response against different SARS-CoV-2 variants of the different vaccine formulations of the study, analysis of SARS-CoV-2 neutralizing antibodies from pig sera was tested. Neutralizing antibodies in serum against SARS-CoV-2 U.K. (alpha; B.1.1.7), South Africa (beta; B.1.351), Brazil (gamma; P.1) and India (delta; B.1.617.2) variants were determined by a pseudovirus-based neutralization assay (PBNA).

For this analysis blood samples were extracted for sera collection on day 35 (14 days post-vaccination after the second administration) for all animals in the different Groups.

The PBNA used is based on an HIV reporter pseudovirus that expresses the S protein of SARS-CoV-2, and the generation of luciferase, as described in Nie J. et al. Quantification of SARS-CoV-2 neutralizing antibody by a pseudotyped virus-based assay. Nat Protoc. 2020 November; 15(11):3699-563715. Four HIV reporter pseudoviruses expressing SARS-CoV-2 S protein and Luciferase were generated, each expressing the corresponding SARS-CoV-2 S protein of a different variant, namely from U.K. (alpha; B.1.1.7) variant, South Africa (beta; B.1.351) variant, Brazil (gamma; P1) variant, and India (delta; B.1.617.2) variant. The difference in the Spike protein between the variants are known and well defined by the Centers for Disease Control and Prevention (CDC) “SARS-CoV-2 Variant Classifications and Definitions”.

For neutralization assay, 200 TCID₅₀ of each variant pseudovirus supernatant was preincubated with serial dilutions of the heat-inactivated serum samples of Groups A to C for 1 h at 37° C. and then added onto ACE2 overexpressing HEK293T cells. After 48 h, cells were lysed with Britelite Plus Luciferase reagent (Perkin Elmer, Waltham, MA, USA). Luminescence was measured for 0-2 s with an EnSight Multimode Plate Reader (Perkin Elmer). Neutralization capacity of the serum samples was calculated by comparing the experimental RLU calculated from infected cells treated with each serum to the max RLUs (maximal infectivity calculated from untreated infected cells) and min RLUs (minimal infectivity calculated from uninfected cells), and expressed as percent neutralization:

% Neutralization=(RLUmax−RLUexperimental)/(RLUmax−RLUmin)*100.

Normalized dose response neutralization curves were fitted to a four-parameter curve with a variable slope using Graph Pad Prism (v8.3.0). All IC₅₀ values are expressed as reciprocal dilution (concentration required to inhibit 50% of infection).

The results show that the prime-boost immunization protocol administered to groups A and B, both induced high neutralising antibody titres against pseudoviruses containing the SARS-CoV-2 variants of U.K. (alpha; B.1.1.7), South Africa (beta; B.1.351), Brazil (gamma; P.1) and India (delta; B.1.617.2) (FIG. 14). Both Groups elicited comparable neutralizing antibody titres against U.K. (P=0.190), Brazil (P=0.412) and India (P=0.111) variants, but Group A based on the fusion dimeric RBD variant SARS-CoV-2 antigen induced significant higher titres against the South Africa variant compared with Group B (commercial vaccine) (P=0.015). Pairwise comparisons using Mann Whitney test; p<0.05.

Therefore, the results clearly show that vaccines based on fusion dimeric RBD variant SARS-CoV-2 antigen induce neutralizing antibodies against different variants, particularly against U.K. (alpha), South Africa (beta), Brazil (gamma) and India (delta) variants. Neutralizing antibody titers generated by the fusion dimeric RBD variant SARS-CoV-2 antigen in vaccinated animals (Group A) were comparable with the commercially available vaccine group (Group B), or even higher in the neutralization assay against the South Africa (beta) variant.

This study shows that a fusion dimeric RBD variant SARS-CoV-2 antigen formulated in an oil-in-water adjuvant presents an optimal balance between immunogenicity and safety, and it performs even better than available commercial vaccines against some of the VOCs, such as the South Africa variant.

### Example 10: Evaluation of Protective Efficacy with a Fusion Dimeric RBD Antigen Against a Heterologous SARS-CoV-2 Infection in Mice

This study evaluates a novel recombinant subunit antigen of SARS-CoV-2. The novel recombinant subunit antigen is a fusion dimeric RBD antigen that contains two monomers, a first monomer comprising a RBD derived from the B.1.351 (South Africa) variant and a second monomer comprising a RBD derived from the B.1.1.7 (UK) variant. This novel recombinant subunit antigen of SARS-CoV-2 is named fusion dimeric RBD variant antigen. The recombinant fusion dimeric RBD variant SARS-CoV-2 antigen is the same as described in Example 5, Groups B to F.

The study evaluates the protective efficacy of this recombinant fusion dimeric RBD variant antigen against COVID-19 disease and the pathogenic outcomes derived from a heterologous SARS-CoV-2 infection in mice. To evaluate the efficacy a challenge model based on the K18-hACE2 transgenic mice was used in this study.

Since the declaration of the pandemic, several challenge models for minor mammalian species have been described. K18-hACE2 transgenic mice are susceptible to the infection with SARS-CoV-2 virus because of the transgenic expression of the ACE2 human receptor, as described in Winkler E.S. et al. SARS-CoV-2 infection of human ACE2-transgenic mice causes severe lung inflammation and impaired function. Nature immunology, 2020, vol. 21, no 11, p. 1327-1335 and Yinda C.K. et al. K18-hACE2 mice develop respiratory disease resembling severe COVID-19. PLoS pathogens, 2021, vol. 17, no 1, p. e1009195. This challenge model, in K18-hACE2 mice, is based on clinical disease and is characterized by moderate clinical, pathological and virological outcomes upon infection with SARS-CoV-2.

A total of 18 K18-hACE2 transgenic mice of 4-5 weeks of age (The Jackson Laboratory, ref. 034860) were allocated in 3 different groups. Each group received a different vaccine formulation as descried below. Group A to C included 6 mice each one. Animals received two doses separated 21 days apart, on Day 0 and Day 21. Each animal received 0.1 ml of the following vaccine formulations per dose by intramuscular route.

The different vaccine formulations administered to mice were the following:
Group A (fusion dimeric RBD variant SARS-CoV-2 antigen, 20 μg): animals in this group received a vaccine formulation comprising 20 μg of the recombinant fusion dimeric RBD variant SARS-CoV-2 antigen. The vaccine was formulated with an oil-in-water adjuvant at a ratio v/v 50% adjuvant and 50% antigen. The oil-in-water adjuvant was formulated as 39 mg/ml of squalene, 4.7 mg/ml of polysorbate 80, 4.7 mg/ml of sorbitan trioleate, 2.64 mg/ml of sodium citrate, and 0.16 mg/ml of citric acid. Thus, a 0.1 ml dose of the vaccine, when mixed at a proportion of 50% adjuvant and 50% antigen, comprises 1.95 mg of squalene, 0.235 mg of polysorbate 80, 0.235 mg of sorbitan trioleate, 0.132 mg of sodium citrate, and 0.008 mg of citric acid.
Group B (fusion dimeric RBD variant SARS-CoV-2 antigen, 10 μg): animals in this group received a vaccine formulation comprising 10 μg of the recombinant fusion dimeric RBD variant SARS-CoV-2 antigen. The vaccine was formulated with the same adjuvant as Group A at a ratio v/v 50% adjuvant and 50% antigen.
Group C (control group): animals in this group received a mock-vaccine comprising PBS.

A challenge was subsequently performed to the animals at day 35 (2 weeks after the second dose) through intranasal infection. Animals received 25 μl per nostril of a solution comprising a titre of 10⁶ TCID₅₀/ml of SARS-CoV-2 virus by using a micropipette. Thus, each animal received a dose of 103 TCID₅₀ SARS-CoV-2 virus. The SARS-CoV-2 isolate used for the challenge was a Wuhan/Hu-1/2019-like isolate, namely the hCoV-19/Spain/CT-IrsiCaixa-JP/2020 (GISAID ID EPI_ISL_471472), designated as Cat02, which was isolated from a human patient from Spain in March 2020. Compared to Wuhan/Hu-1/2019 strain, Cat02 isolate has the D614G point mutation in the Spike protein.

The primary endpoint reporting the protective capacity of the vaccine candidates is weight loss and/or mortality post-challenge.

Thus, to assess the protective efficacy after challenge with the different vaccine formulations of the study, weight and mortality was monitored during one week after challenge (day 42). Unprotected animals vulnerable to SARS-CoV-2 virus are expected to show weight loss at the end of the study. For this reason, weight was monitored daily during the challenge stage.

Secondary endpoints were also monitored, including viral spread throughout the organism (assessed by RT-PCR and viral titration), especially within those organs and tissues belonging to the respiratory system, the main target of the viral infection and replication.

The results surprisingly show that all the animals that received a vaccine comprising the recombinant fusion dimeric RBD variant SARS-CoV-2 antigen, either at 10 or 20 μg/dose, survived 7 days after the experimental infection (Groups A and B). On the other hand, the control group (Group C) resulted in 100% of mortality. All animals in the control Group died between days 5-6 after the challenge (FIG. 15).

Furthermore, none of the animals that received the vaccine comprising the recombinant fusion dimeric RBD variant SARS-CoV-2 antigen (Groups A and B), either at 10 or 20 μg/dose presented weight loss after the experimental infection. Contrary, a clear weight loss was observed after the experimental infection in all animals included in the control group (mock-vaccinated with PBS, Group C).

Therefore, the results clearly show that the recombinant fusion dimeric RBD variant SARS-CoV-2 antigen is able to protect against heterologous SARS-CoV-2 infection, and also prevent clinical signs of SARS-CoV-2 infection, such as weight loss and mortality.

### Example 11: Assessment of Immunogenicity and Safety of a Booster Vaccination with a Recombinant Protein Vaccine Composition Based on a Fusion Dimeric RBD Variant Against SARS-CoV-2 in Adult Subjects

This study provides a summary of clinical data obtained after assessing the immunogenicity and safety of a booster dose of a novel fusion heterodimer RBD variant SARS-CoV-2 antigen composition (named PHH-1V) in healthy adult subjects fully vaccinated against COVID-19 with two doses of a reference vaccine such as Comirnaty® (BioNTech Manufacturing GmbH). The study is a Phase 2b, double-blind, randomized, active-controlled, multicenter, non-inferiority trial to determine and compare the immunogenicity and safety of PHH-1V at baseline (Day 0) and Day 14 versus subjects who have received complete vaccination, including homologous booster, with the Pfizer-BioNTech vaccine at least 182 days and with a maximum of 365 days before booster vaccination. Approximately 602 adults aged 18 years old and above, were designated to be randomized to either PHH-1V or Comirnaty® group.

Overall, 752 subjects were finally assessed in the efficacy study. They were randomly assigned 2:1 following two different treatment groups. Cohort 1 (n=504) received a single booster dose of a 0.5 ml vaccine (PHH-1V) by intramuscular route on Day 0. One dose (0.5 ml) of PHH-1V vaccine comprises 40 μg of the novel fusion heterodimer RBD variant SARS-CoV-2 antigen which is based on a first monomer comprising an RBD derived from the B.1.351 SARS-CoV-2 variant and a second monomer comprising an RBD derived from the B.1.1.7 SARS-CoV-2 variant produced, as in Example 3, by recombinant DNA technology using a plasmid expression vector in a CHO cell line optimized for stable production. PHH-1V is also adjuvanted with 0.25 ml of an adjuvant containing per 0.5 ml dose: squalene (9.75 mg), polysorbate 80 (1.175 mg), sorbitan trioleate (1.175 mg), sodium citrate (0.66 mg) and citric acid (0.04 mg). The recombinant fusion heterodimer RBD variant SARS-CoV-2 antigen is the same as described in Example 5 (Groups B to F). Cohort 2 (n=248) received a single booster dose of 0.3 ml Comirnaty® vaccine (BioNTech Manufacturing GmbH) by intramuscular route on Day 0.

Accordingly, subjects received a single booster dose according to treatment assignment on Day 0.

Each subject was followed for 52 weeks (364 days) after the administration of the booster vaccination on Day 0. The total clinical study duration for each subject was up to 56 weeks.

The immunogenicity of the booster vaccination with both vaccines was evaluated at baseline and at Day 14 day after receiving the booster vaccination. The neutralization antibody titres against VOC variants such as the Beta (B.1.351), Delta (B.1.617.2) and Omicron (B.1.1.529) SARS-CoV-2 variants was measured as half maximal inhibitory concentration (IC50) by a pseudovirion-based neutralization assay (PBNA), as described in Example 2, and reported as the geometric mean titer (GMT) for the treatment group (Table 1). The geometric mean fold-rise (GMFR) in binding neutralizing antibody titres from baseline (Day 0) and Day 14 was also determined. The percentage of subjects that after a booster dose have a 4-fold change in binding antibodies titre from baseline (Day 0) and Day 14 was also calculated.

The GMT for treatment means and the geometric mean fold rise (GMFR) ratio were estimated using LS Means (Least Square Means) from the fitted model MMRM (Mixed model repeated measures) on the log 10 scale and back-transformed.

TABLE 1
Geometric mean titres (GMT) of neutralizing antibodies
against Variant of Concern (VOC) at Baseline (Day
0) and Day 14: Cohort 1 and Cohort 2.

| Treatment | Day | Beta | Delta | Omicron |
|---|---|---|---|---|
| Cohort 1/(PHH-1V) | Day 0 | 66.92 | 44.88 | 32.87 |
| | Day 14 | 4352.89 | 11471.78 | 2063.44 |
| Cohort 2/(Comirnaty ®) | Day 0 | 60.76 | 41.17 | 29.06 |
| | Day 14 | 2665.33 | 1487.11 | 1222.00 |

After 14 days of treatment with PHH-1V or Comirnaty® the following results were obtained:

SARS-CoV-2 Beta variant (B.1.351): At baseline, log 10 transformed geometric mean neutralizing antibody levels were similar between Cohort 1 and Cohort 2 (66.92 and 60.76 respectively). Neutralizing antibody levels on Day 14 increased in both cohorts with a greater increase in the PHH-1V vaccine group (4352.89) compared to the Comirnaty® vaccine group (2665.33).

SARS-CoV-2 Delta variant (B.1.617.2): At baseline, log 10 transformed geometric mean neutralizing antibody levels were similar between Cohort 1 and Cohort 2 (44.88 and 41.17 respectively). Neutralizing antibody levels on Day 14 increased in both cohorts to similar levels: PHH1-V vaccine group (1471.78), Comirnaty® vaccine group (1487.11).

SARS-CoV-2 Omicron variant (B.1.1.529): At baseline, log 10 transformed geometric mean neutralizing antibody levels were similar between Cohort 1 and Cohort 2 (32.87 and 29.06 respectively). Neutralizing antibody levels on Day 14 increased in both cohorts with a greater increase in the PHH-1V vaccine group (2063.44) compared to the Comirnaty® vaccine group (1222.00).

Results from neutralizing antibody titres at day 14 after booster vaccination clearly demonstrate that a booster dose of a vaccine based on the novel fusion heterodimer RBD variant SARS-CoV-2 antigen of PHH-1V induces high levels of neutralizing antibodies against different SARS-CoV-2 variants of concern (VOC).

Surprisingly the neutralizing antibody titers (GMT) induced by the novel fusion heterodimer RBD antigen of PHH-1V are higher than the neutralizing antibodies induced by the reference Covid-19 vaccine Comirnaty® against Beta (1.351) and Omicron (B.1.1.529) variants and results in similar high levels for the Delta variant. In the same way results from the PBNA assay of PHH-1V against Wuhan SARS-CoV-2 confirm high neutralizing antibodies titers against this variant as well. Overall, the results demonstrate an increased and better immunogenicity response of PHH-1V against SARS-CoV-2 variants of concern compared to the comparator group that received Comirnaty®.

Likewise, the fold-rise obtained in neutralizing antibodies titers on Day 14 from Baseline, confirms previous data.

Accordingly, the geometric mean fold rise (GMFR) ratio in neutralizing antibody titers for the Beta and Omicron SARS-CoV-2 variants demonstrate superiority of the Cohort 1/PHH-1V over the Cohort 2/comparator vaccine Comirnaty®, with a GMFR ratio of 0.69 (p-value 0.0003) for the Beta SARS-CoV-2 variant and 0.68 (p-value 0.0001) for the Omicron SARS-CoV-2 variant. For the delta SARS-CoV-2 variant, results from the fold-rise in neutralizing antibodies titers demonstrate non-inferiority of the Cohort 1/PHH-1V to the Cohort 2/Comirnaty®, with a GMFR ratio of 1.11 (p-value 0.2446).

The fold-rise mean in neutralizing antibody titres demonstrates an increased and better immunogenicity of the PHH-1V vaccine, comprising the novel fusion heterodimer RBD variant SARS-CoV-2 antigen, compared to the comparator group (Cohort 2, Comirnaty®) against novel SARS-CoV-2 variants of concern.

To evaluate the SARS-CoV-2-specific T-cell responses, different peptide pools of overlapping SARS-CoV-2 peptides each encompassing the SARS-CoV-2 regions S (two pools), RBD, nucleoprotein, membrane, and envelope were used.

T-cell responses were analyzed at Baseline and at Day 14 as present or absent and reported as the number and proportion of subjects responding to each peptide pool and for each timepoint. The total ELISpot responses were described as the sum of SFC/106 PBMC (peripheral blood mononuclear cell) of all positive responses per peptide pool, after subtraction of background. Each subject was classified as a responder if there was at least one positive against any of the SARS-CoV-2 peptides pools at any time, and non-responder if ELISpot responses were all negative.

In addition, intracellular cytokine staining (ICS) based T-cell assay was determined at different timepoints. ICS assays included Th1/Th2 pathways (e.g., IL-2, IL-4, INFγ) CD4+ and CD8+ T-cell determinations using flow cytometry. CD4+ and CD8+ T-cell response was measured at Baseline at Day 14.

An ICS was considered positive if the percentages of cytokine-positive cells in the stimulated samples were three times more than the values obtained in the unstimulated controls and if the background-subtracted magnitudes were higher than 0.02%. Each subject was classified as a responder if there were at least one positive IFN-γ ICS response against any of the SARS-CoV-2 peptide pools at determined timepoints and as a non-responder if responses at these timepoints were all negative.

T-cell mediated immune response against SARS-CoV-2 was assessed after in vitro peptide stimulation of peripheral blood mononuclear cells (PBMC) followed by IFN-γ enzyme linked immune absorbent spot (IFN-γ ELISpot) in a subset group of subjects randomly divided in Cohort 1 and Cohort 2, wherein the subjects of both Cohorts were previously vaccinated with two doses of Comirnaty®, and then boosted with either one dose of PHH-1V (Cohort 1) or one dose of Comirnaty® (Cohort 2).

Different peptide pools for overlapping SARS-CoV-2 Spike protein were used, i.e., Spike SA and Spike SB pools, a pool of RBD alpha, RBD beta, and RBD delta variants. In particular, the peptides used for the stimulation of PBMC were: SPIKE SA (194 peptides overlapping S1-2016 to S1-2196 region of the Spike protein), SPIKE SB (168 peptides overlapping the S1-2197 to S2-2377 region of the Spike protein), RBD alpha variant (84 peptides overlapping the RBD region of the SARS-CoV-2 alpha variant) and RBD beta variant (84 peptides overlapping the RBD region of the SARS-CoV-2 beta variant), and RBD delta variant (84 peptides overlapping the RBD region of the SARS-CoV-2 delta variant).

The results of the T-cell response showed a significant increase of IFN-γ producing lymphocytes upon in vitro re-stimulation with peptide pools at 2 weeks post-boost in comparison with the levels observed at baseline. Interestingly, the booster dose with PHH-1V vaccine (Cohort 1) induced significant activation of CD4+ T cells expressing IFN-γ upon re-stimulation with pools of RBD peptides from alpha, beta and delta Variants of Concern. In addition, remarkably this response was stronger compared to those subjects boosted with Comirnaty® (Cohort 2). No IL-4 expression was detected in the activated CD4+ T cells after the in vitro re-stimulation, for the PHH-1V booster vaccine suggesting that the vaccine induced a Th1-biased T-cell response. Furthermore, the heterologous boost with the PHH-1V (Cohort 1) vaccine was proven to induce the activation of CD8+ T cells expressing IFN-γ.

For assessing the tolerability and safety of PHH-1V, the number, percentage and characteristics of solicited local reactions and systemic events from Day 0 through Day 7 after vaccination was evaluated. In general, local and systemic adverse events were more frequently reported by subjects that received Comirnaty® comparator vaccine (Cohort 2), the recorded percentage in all cases was higher in Cohort 2 (Comirnaty®) than in Cohort 1 (PHH-1V vaccine).

The most frequently reported solicited local reactions from Day 0 through to Day 7 were pain and tenderness. Pain was reported by 51.1% of subjects in Cohort 1 and 68.8% of subjects in Cohort 2. Tenderness was reported by 48.5% of subjects in Cohort 1 and 63.5% of subjects in Cohort 2. The most frequently reported solicited systemic adverse event from Day 0, 12 hours through to Day 2 was fatigue. Fatigue was reported more frequently in Cohort 2 (Comirnaty®), on 12 hours (18.7%), Day 1 (35.3%) and Day 2 (13.1%) compared to Cohort 1 (PHH-1V vaccine) (16.0%, 16.0% and 7.6%, respectively).

Overall, PHH-1V consistently shows a good safety profile and high and increased levels of neutralizing antibodies against different SARS-CoV-2 variants of concern (VOCs) in adult subjects. Remarkably, PHH-1V shows a high and increased neutralizing titer over the vaccine comparator against omicron SARS-CoV-2 variant, even given the heavily mutated Spike protein observed for this new B.1.1.529 variant. These results support that the PHH-1V vaccine candidate based on the novel fusion heterodimer SARS-CoV-2 variant antigen with an increased and better immunogenicity over the vaccine comparator. Accordingly, the results support that PHH-1V is efficacious against different SARS-CoV-2 variants and has the potential to confer protection against future SARS-CoV-2 variants of concern.

### Example 12: Assessment of Native-Like Structure by Surface Plasmon Resonance

As shown and discussed in previous Examples, the PHH-1 vaccine candidate elicits a robust humoral response with high titers of neutralizing antibodies. Generating native-like protein subunit vaccines is of paramount importance as the native structure is a strong indicative of a better capacity to elicit neutralizing antibodies with higher affinity to the antigen present in the wild-type virus. To confirm that the fusion heterodimer RBD variant SARS-CoV-2 antigen has a native-like structure, a surface plasmon resonance (SPR) analysis by ACROBiosystems was performed with human ACE2. The Fc tagged ACE2 (AC2-H5257, ACROBiosystems) was immobilised in a Series S Sensor Chip CM5 (Cytiva) on a Biacore T200 (Cytiva) using the Human Antibody Capture Kit (Cytiva). The affinity measure was obtained using 8 different RBD heterodimer concentrations. The antigen structure simulations were performed with UCSF ChimeraX.

### Detailed Materials and Methods:

### Human Running Buffer:

I×HEPES (10 mM HEPES, 150 mM NaCl. 3 mM EDTA), with 0.005% Tween-20. pH7.4. Human Antibody Capture Kit (BR-1008-39, Cytiva): Anti-human IgG (Fc) antibody (500 μg/mL), Immobilization buffer (10 mM Sodium Acetate. pH5.0). Regeneration buffer (3 M magnesium chloride).

### Chip Preparation

Dilute the Anti-Human IgG (Fc) antibody to 25 μg/mL in Immobilization buffer 10 mM Sodium Acetate. pH5.0 (add 50 μL Anti-Human IgG (Fc) antibody into 950 μL Immobilization buffer for eight channels). The activator is prepared by mixing 400 mM EDC and 100 mM NHS (GE) immediately prior to injection. The CM5 sensor chip is activated for 420 s with the mixture at a flow rate of 10 μL/min. 25 μg/mL of Anti-Human IgG (Fc) antibody in Immobilization buffer 10 mM Sodium Acetate (pH5.0) is then injected to FC2 sample channel for 420 s at a flow rate of μL/min, and typically result in immobilization levels of 9000 to 14000 RU. The chip is deactivated by 3 M magnesium chloride (Cytiva) at a flow rate of 20 μL/min for 30 s. The reference surface FC1 channel should be prepared in the same way as the active surface FC2 channel. (Refer to GE Human antibody Capture Kit Instruction 29237227 AB).

### Ligand Protein Reconstitution

Reconstitute Human ACE2/ACEH Protein following the COA. To avoid surface adsorption loss and inactivation, the reconstituted protein must NOT be aliquoted to less than 10 μg per vial, see Table 2:

TABLE 2
Reconstitution information of Ligand proteins

| | | Concentration |
|---|---|---|
| Name | Cat. No. | (mg/mL) |
| Human ACE2/ACEH | AC2-H5257 | 0.355 |
| Protein, Fc Tag | | |

### Ligand Captured

Diluted Human ACE2 to 10 μg/mL with Running Buffer and then injected to sample channel (FC2) at a flow rate of 10 μL/min to reach a capture level of about 300 RU. the reference channel (FC 1) does not need Ligand Capturing step.

### Ran Analyte by Multi-Cycle Method

Diluted Client's samples with the Running Buffer to Corresponding concentration (Table 6). The diluted samples are injected to FC1-FC2 of channel at a flow rate of 30 μL/min for an association phase of 90 s, followed by 210 s dissociation.

The association and dissociation process are all handling in the Running Buffer. After each cycle of interaction analysis, the sensor chip surface should be regenerated completely with 3 M magnesium chloride as injection buffer at a flow rate of 20 μL/min for 30 s to remove the ligand and any bound analyte.

TABLE 3
Parameter of affinity test between antibody
samples binding to human ACE2 protein.

| | | Analyte | | |
|---|---|---|---|---|
| Ligand | Analyte | conc. | Association(s) | Dissociation(s) |
| Human ACE2/ACEH | PHH1-V | 0.195-25 nM | 90 | 210 |
| Protein Fc Tag | | | | |

### Other Details

The whole processes were handling in Running Buffer. The other buffers used in SPR Assay process were the same as the injection buffer, which was placed in the rack tray of sample compartment.

### Results

### Kinetic Affinity (SPR)

Analyzed the affinity by Biacore Insight Evaluation in Biacore T200. The reference channel (FC1) was used for background subtraction.

TABLE 4
Summary of affinity assay between antibody
samples and human ACE2 protein.

| Method | Ligand | KD (M) |
|---|---|---|
| Human IgG | Human ACE2/ACEH | 9.85E−11 |
| (Fc) Capture | Protein Fc Tag | |

### Conclusions

As shown in Table 4, the fusion heterodimer RBD variant SARS-CoV-2 antigen showed an affinity constant for hACE2 of 98.5 pM, indicating an outstanding binding affinity with its natural ligand, which is a clear sign of native-like structure and which explains the potent neutralizing antibodies elicited against the different SARS-CoV-2 virus variants.

### CLAUSES

The following clauses are comprised in the present invention:
1. A protein subunit vaccine comprising at least one antigen comprising at least two monomers from at least one variant of severe acute respiratory syndrome coronavirus 2 (SARS-CoV-2), wherein each of the monomers are selected from the group consisting of the S1 subunit of the Spike protein or the receptor-binding domain (RBD) of the Spike protein, or any immunogenic fragments thereof, and wherein the two monomers are chemically bound to each other, optionally through a linker, forming a dimer.
2. The protein subunit vaccine according to clause 1, wherein the antigen comprises or consists of two monomers, wherein both monomers comprise or consist of the receptor-binding domain (RBD) of the Spike protein from at least one variant of severe acute respiratory syndrome coronavirus 2 (SARS-CoV-2).
3. The protein subunit vaccine according to clause 2, wherein said receptor-binding domain (RBD) of the Spike protein has at least 90% sequence identity, over its full length, with any of SEQ ID NO 1, SEQ ID NO 3 or SEQ ID NO 4.
4. The protein subunit vaccine according to any of clauses 1 to 3, wherein the at least one variant of severe acute respiratory syndrome coronavirus 2 (SARS-CoV-2) is selected from the group consisting of Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant), or Linage B.1.1.7 (United Kingdom variant), or any combination thereof.
5. The protein subunit vaccine according to any of clauses 1 to 4, wherein the dimer is a fusion dimer comprising or consisting of two monomers, wherein the two monomers are part of a single polypeptide.
6. The protein subunit vaccine according to clause 5, wherein the fusion dimer consists of a first RBD monomer from a first SARS-CoV-2 variant and a second RBD monomer from a different second SARS-CoV-2 variant.
7. The protein subunit vaccine according to clause 6, wherein the fusion dimer consists of a first monomer derived from the Linage B.1.351 (South African SARS-CoV-2 variant), and a second monomer derived from the Linage B.1.1.7 (United Kingdom SARS-CoV-2 variant).
8. The protein subunit vaccine according to clause 7, wherein the fusion dimer has at least 90% sequence identity, over its full length, with SEQ ID NO 5.
9. The protein subunit vaccine according to clause 8, wherein the fusion dimer comprises or consists of SEQ ID NO 5.
10. The protein subunit vaccine according to any of clauses 1 to 4, wherein the dimer consists of a non-fusion dimer comprising or consisting of two monomers, wherein the two monomers are bound by reversible bonds.
11. The protein subunit vaccine according to clause 10, wherein the non-fusion dimer consists of a first monomer and a second monomer, both derived from the Wuhan-Hu-1 seafood market pneumonia virus isolate (GenBank accession number: MN908947).
12. The protein subunit vaccine according to any of clauses 10 and 11, wherein the first and the second monomers have at least 90% sequence identity, over its full length, with SEQ ID NO 1.
13. The protein subunit vaccine according to clause 12, wherein the non-fusion dimers comprise or consist of SEQ ID NO 1.
14. The protein subunit vaccine according to any of clauses 1 to 13, wherein the protein subunit vaccine comprises a total amount of antigen per dose of between 5 to 50 μg.
15. The protein subunit vaccine according to any of clauses 1 to 14, further comprising at least an adjuvant.
16. The protein subunit vaccine according to clause 15, wherein the adjuvant comprises about 10 to 60 mg/ml of squalene, 1 to 6 mg/ml of polysorbate 80, 1 to 6 mg/ml of sorbitan trioleate, 0.5 to 6 mg/ml of sodium citrate, and 0.01 to 0.5 mg/ml of citric acid.
17. The protein subunit vaccine according to any of clauses 1 to 16, wherein the protein subunit vaccine further comprises Monophosphoryl lipid A (MPLA) and/or C₉₂O₄₆H₁₄₈ (QS-21) as immunostimulants.
18. A protein subunit vaccine as defined in any of clauses 1 to 17, for use in generating an immunogenic and/or protective immune response against at least one variant of the Severe acute respiratory syndrome coronavirus 2 (SARS-CoV-2) virus in a subject in need thereof.
19. The protein subunit vaccine for use according to clause 18, wherein the protein subunit vaccine is administered to the subject in need thereof in a single dose or multiple doses, preferably in two doses.
20. The protein subunit vaccine for use according to any of clauses 18 and 19, wherein the protein subunit vaccine is administered to the subject in need thereof in a schedule comprising a first dose or priming and a second dose or boosting.
21. The protein subunit vaccine for use according to any of clauses 18 to 20, wherein the second dose is administered one, preferably two, three or four weeks after the first dose.
22. The protein subunit vaccine for use according to any of clauses 18 to 21 wherein said protein subunit vaccine is administered intramuscularly or subcutaneously.
23. A kit comprising at least one, preferably two, or more doses of the protein subunit vaccine as defined in any of clauses 1 to 17.
24. A protein subunit vaccine comprising at least one antigen characterized in that it comprises at least one monomer from at least one variant of severe acute respiratory syndrome coronavirus 2 (SARS-CoV-2), wherein the at least one monomer is selected from the group consisting of the S1 subunit of the Spike protein or the receptor-binding domain (RBD) of the Spike protein, or any immunogenic fragments thereof, and wherein the protein subunit vaccine further comprises at least one adjuvant, wherein said adjuvant comprises about 10 to 60 mg/ml of squalene, 1 to 6 mg/ml of polysorbate 80, 1 to 6 mg/ml of sorbitan trioleate, 0.5 to 6 mg/ml of sodium citrate, and 0.01 to 0.5 mg/ml of citric acid.
25. The protein subunit vaccine according to clause 24, wherein the at least one monomer comprises or consists of a recombinant receptor-binding domain (RBD) of the Spike protein or an immunogenic fragment thereof.
26. The protein subunit vaccine according to clause 25, wherein the receptor-binding domain (RBD) has at least 90% sequence identity, over its full length, with any of SEQ ID NO 1, SEQ ID NO 3 or SEQ ID NO 4.
27. The protein subunit vaccine according to clause 24, wherein the at least one monomer is a recombinant S1 subunit of the Spike protein or an immunogenic fragment thereof.
28. The protein subunit vaccine according to clause 27, wherein the S1 subunit has at least 90% sequence identity, over its full length, with SEQ ID NO 2.
29. The protein subunit vaccine according to any of clauses 24 to 28, wherein the protein subunit vaccine further comprises Monophosphoryl lipid A (MPLA) and/or C₉₂O₄₆H₁₄₈ (QS-21) as immunostimulants.
30. The protein subunit vaccine according to any of clauses 25 and 26, wherein the at least one monomer is RBD of the Spike protein and the immunostimulant is Monophosphoryl lipid A (MPLA).
31. The protein subunit vaccine according to any of clauses 27 and 28, wherein the at least one monomer is the S1 subunit of the Spike protein and the immunostimulant is Monophosphoryl lipid A (MPLA).
32. The protein subunit vaccine according to any of clauses 24 to 31, wherein the at least one antigen is a monomer or a multimer, preferably a dimer.
33. The protein subunit vaccine according to clause 24, wherein the protein subunit vaccine comprises at least one antigen comprising or consisting of two monomers from at least one variant of severe acute respiratory syndrome coronavirus 2 (SARS-CoV-2), wherein each of said monomers are selected from the group consisting of the S1 subunit of the Spike protein or the receptor-binding domain (RBD) of the Spike protein, or any immunogenic fragments thereof, and wherein the two monomers are chemically bound to each other, optionally through a linker, forming a dimer.
34. The protein subunit vaccine according to clause 33, wherein both monomers are the RBD of the Spike protein from at least one variant of severe acute respiratory syndrome coronavirus 2 (SARS-CoV-2).
35. The protein subunit vaccine according to any of clauses 33 and 34, wherein a first monomer is from a first SARS-CoV-2 variant and a second monomer is from a different second SARS-CoV-2 variant.
36. The protein subunit vaccine according to any of clauses 24 to 35, wherein the at least one variant of severe acute respiratory syndrome coronavirus 2 (SARS-CoV-2) is selected from the group consisting of Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) or Linage B.1.1.7 (United Kingdom variant), or any combination thereof.
37. The protein subunit vaccine according to any of clauses 33 to 35, wherein the dimer is a fusion dimer that comprises or consists of a first monomer derived from the Linage B.1.351 (South African SARS-CoV-2 variant), and a second monomer derived from the Linage B.1.1.7 (United Kingdom SARS-CoV-2 variant), wherein the two monomers of the fusion dimer are part of a single polypeptide.
38. The protein subunit vaccine according to clause 37, wherein the fusion dimer has at least 90% sequence identity, over its full length, with SEQ ID NO 5.
39. The protein subunit vaccine according to any of clauses 33 to 35, wherein the dimer is a non-fusion dimer that comprises or consists of a first monomer and a second monomer, both derived from Wuhan-Hu-1 seafood market pneumonia virus isolate (GenBank accession number: MN908947), wherein the two monomers of the non-fusion dimer are bound by reversible bonds.
40. The protein subunit vaccine according to clause 39, wherein the first and the second monomers have at least 90% sequence identity, over its full length, with SEQ ID NO 1.
41. The protein subunit vaccine according to clauses 24 to 36, 39 and 40, wherein the protein subunit vaccine comprises a mixture of at least a monomeric RBD antigen and at least a dimeric RBD antigen, wherein at least 45% of the total antigen comprised in the protein subunit vaccine is a dimeric RBD antigen.
42. The protein subunit vaccine according to any of clauses 33 to 41, wherein the protein subunit vaccine further comprises Monophosphoryl lipid A (MPLA) and/or C₉₂O₄₆H₁₄₈ (QS-21) as immunostimulants.
43. The protein subunit vaccine according to any of clauses 24 to 42, wherein the protein subunit vaccine comprises a total amount of antigen per dose of between 5 to 50 μg.
44. The protein subunit vaccine according to any of clauses 24 to 43, wherein the adjuvant comprises about 39 mg/ml of squalene, 4.7 mg/ml of polysorbate 80, 4.7 mg/ml of sorbitan trioleate, 2.64 mg/ml of sodium citrate, and 0.16 mg/ml of citric acid.
45. The protein subunit vaccine according to any one of clauses 24 to 44, for use in generating an immunogenic and/or protective immune response against at least one variant of the Severe acute respiratory syndrome coronavirus 2 (SARS-CoV-2) virus in a subject in need thereof.
46. The protein subunit vaccine for use according to clause 45, wherein the protein subunit vaccine is administered to the subject in need thereof in a single dose or multiple doses, preferably in two doses.
47. The protein subunit vaccine for use according to clause 46, wherein the protein subunit vaccine is administered to the subject in need thereof in a schedule comprising a first dose or priming and a second dose or boosting.
48. The protein subunit vaccine for use according to any of clauses 45 to 47, wherein the second dose is administered one, preferably two, three or four weeks after the first dose.
49. The protein subunit vaccine for use according to any of clauses 45 to 48 wherein said protein subunit vaccine is administered intramuscularly or subcutaneously.
50. A kit comprising at least one, preferably two, or more doses of the protein subunit vaccine as defined in any one of clauses 24 to 45.

1. A protein subunit vaccine comprising at least one antigen characterized in that it comprises at least one monomer from at least one linage of severe acute respiratory syndrome coronavirus 2 (SARS-CoV-2), wherein the at least one monomer is the receptor-binding domain (RBD) of the Spike protein, and wherein the protein subunit vaccine further comprises at least one adjuvant, wherein said adjuvant comprises about to 60 mg/ml of squalene, 1 to 6 mg/ml of polysorbate 80, 1 to 6 mg/ml of sorbitan trioleate, 0.5 to 6 mg/ml of sodium citrate, and 0.01 to 0.5 mg/ml of citric acid.

2. The protein subunit vaccine according to claim 1, wherein the receptor-binding domain (RBD) has at least 90% sequence identity, over its full length, with any of SEQ ID NO 1, SEQ ID NO 3 or SEQ ID NO 4.

3. The protein subunit vaccine according to claim 1, wherein the protein subunit vaccine comprises at least one antigen comprising two monomers from at least one linage of severe acute respiratory syndrome coronavirus 2 (SARS-CoV-2), wherein each of said monomers are the receptor-binding domain (RBD) of the Spike protein, and wherein the two monomers are chemically bound to each other.

4. The protein subunit vaccine according to claim 1, wherein the at least one linage of severe acute respiratory syndrome coronavirus 2 (SARS-CoV-2) is selected from the group consisting of Wuhan-Hu-1 seafood market pneumonia virus isolate (GenBank accession number: MN908947), Linage B.1.1.28 (Brazilian variant), Linage B.1.351 (South African variant), Linage B.1.427 or Linage B.1.429 (California variant), Linage B.1.617 (Indian variant) and Linage B.1.1.7 (United Kingdom variant), and any combination thereof.

5. The protein subunit vaccine according to claim 3, wherein the dimer is a fusion dimer that consists of a first monomer derived from the Linage B.1.351 (South African SARS-CoV-2 variant), and a second monomer derived from the Linage B.1.1.7 (United Kingdom SARS-CoV-2 variant), wherein the two monomers of the fusion dimer are part of a single polypeptide.

6. The protein subunit vaccine according to claim 5, wherein the fusion dimer consists of SEQ ID NO 5.

7. The protein subunit vaccine according to claim 3, wherein the dimer is a non-fusion dimer that consists of a first monomer and a second monomer, both derived from Wuhan-Hu-1 seafood market pneumonia virus isolate (GenBank accession number: MN908947), wherein the two monomers of the non-fusion dimer are bound by reversible bonds.

8. The protein subunit vaccine according to claim 1, wherein the protein subunit vaccine comprises a mixture of at least a monomeric RBD antigen and at least a dimeric RBD antigen, wherein at least 45% of the total antigen comprised in the protein subunit vaccine is a dimeric RBD antigen.

9. The protein subunit vaccine according to claim 1, wherein the protein subunit vaccine further comprises Monophosphoryl lipid A (MPLA) and/or C₉₂O₄₆H₁₄₈ (QS-21) as immunostimulants.

10. A method of generating an immunogenic and/or protective immune response against at least one linage of the severe acute respiratory syndrome coronavirus 2 (SARS-CoV-2) virus comprising administering the protein subunit vaccine of claim 1 in a subject in need thereof.

11. The method of claim 10 wherein the protein subunit vaccine is administered to the subject in need thereof in a single dose or multiple doses.

12. The method of claim 10, wherein the protein subunit vaccine is administered to the subject as a booster.

13. A kit comprising at least one or more doses of the protein subunit vaccine as defined in claim 1.

14. The method of claim 10, wherein the protein subunit vaccine is administered to the subject in need thereof in two doses.

15. The kit of claim 13, comprising two or more doses of the protein subunit vaccine.

16. The protein subunit vaccine according to claim 3, wherein the two monomers are chemically bound to each other through a linker, thereby forming a dimer.
